(19) United States
(12) Patent Application Publication
(10) Pub. No.: US 2025/0292904 A1
(43) Pub. Date: Sep. 18, 2025

(54) MODEL GENERATION APPARATUS FOR THERAPEUTIC PREDICTION AND ASSOCIATED METHODS AND MODELS

(71) Applicants: GE Healthcare Limited, Chalfont St. Giles (GB); Vanderbilt University, Nashville, TN (US)

(72) Inventors: Jan Wolber, Chalfont St Giles (GB); Eszter Katalin Csernai, Budapest (HU); Zoltán Kiss, Budapest (HU); Levente Lippenszky, Budapest (HU); Gergely Horváth, Budapest (HU); Travis Osterman, Nashville, TN (US); Ben Ho Park, Nashville, TN (US); David Samuel Smith, Nashville, TN (US); Daniel Fabbri, Nashville, TN (US); Michele LeNoue-Newton, Nashville, TN (US); Kathleen Mittendorf, Nashville, TN (US)

(21) Appl. No.: 18/859,511

(22) PCT Filed: Apr. 26, 2023
(86) PCT No.: PCT/US2023/020068

§ 371 Date: Oct. 23, 2024

(30) Foreign Application Priority Data
Jun. 3, 2022 (WO) PCT/US2022/032075
Jun. 3, 2022 (WO) PCT/US2022/032084

(51) Int. Cl. G16H 50/20

(52) U.S. Cl. CPC G16H 50/20

(57) ABSTRACT

Methods, apparatus, systems, and articles of manufacture are disclosed for generation and application of models for therapeutic prediction and processing. A balance of precision and recall can be applied to at least one of a toxicity-related model or an efficacy-related model to configure immunotherapy treatment of a patient and/or cohort of patients. Model output can be evaluated differently depending on a determine patient selection criterion to trigger different actions.

### CROSS-REFERENCE TO RELATED APPLICATION

This patent claims the benefit of priority to International Application No. PCT/US2022/032075, filed Jun. 3, 2022, International Application No. PCT/US2022/032084, filed Jun. 3, 2022, and International Application No. PCT/US2023/020068, filed Apr. 26, 2023, which claim priority to U.S. Provisional Patent Application Ser. No. 63/335,215, filed Apr. 26, 2022, each of which is hereby incorporated herein by reference in its entirety for all purposes.

### FIELD OF THE DISCLOSURE

This disclosure relates generally to model generation and processing and, more particularly, to generation and application of models for therapeutic prediction and processing.

### BACKGROUND

The statements in this section merely provide background information related to the disclosure and may not constitute prior art.

Immunotherapy can be used to provide effective treatment of cancer for some patients. For those patients, immunotherapy can provide higher efficacy and less toxicity than other therapies. Immunotherapy can include targeted antibodies and immune checkpoint inhibitors (ICI), cell-based immunotherapies, immunomodulators, vaccines, and oncolytic viruses to help the patient's immune system target and destroy malignant tumors. However, in some patients, immunotherapy can cause toxicity and/or other adverse side effect. Immunotherapy side effects may be different from those associated with other cancer treatments because the side effects result from an overstimulated or misdirected immune response rather than the direct effect of a chemical or radiological therapy on cancer and healthy tissues. Immunotherapy toxicities can include conditions such as colitis, hepatitis, pneumonitis, and/or other inflammation that can pose a danger to the patient. Immunotherapies also elicit differing (heterogenous) efficacy responses in different patients. Some patients who respond efficaciously may be affected by toxicities as well. As such, evaluation of immunotherapy remains unpredictable with potential for tremendous variation between patients.

### BRIEF DESCRIPTION OF THE DRAWINGS

FIG. 1 illustrates an example model generation apparatus.

FIGS. 2-14 show flow diagrams illustrating execution of instructions to drive operations using the example model generation apparatus of FIG. 1.

FIG. 15 illustrates an example immunotherapy prediction apparatus.

FIGS. 16-18 illustrate flow diagrams of example methods for processing data with one more models according to the example immunotherapy prediction apparatus of FIG. 15.

FIG. 19 illustrates an example timeline from aggregation of data and formation of associated features to model development with respect to first time of immunotherapy treatment.

FIGS. 20-24 depict example precision/recall graphs illustrating characteristics of example models.

FIG. 25 shows an example infrastructure and organization of patient health data from patient health records to features that can be used for model training, testing, and validation.

FIG. 26 is a block diagram of an example processing platform including processor circuitry structured to execute example machine readable instructions and/or the example operations.

FIG. 27 is a block diagram of an example implementation of the processor circuitry of FIG. 26.

FIG. 28 is a block diagram of another example implementation of the processor circuitry of FIG. 26.

FIG. 29 is a block diagram of an example software distribution platform (e.g., one or more servers) to distribute software (e.g., software corresponding to example machine readable instructions) to client devices associated with end users and/or consumers (e.g., for license, sale, and/or use), retailers (e.g., for sale, re-sale, license, and/or sub-license), and/or original equipment manufacturers (OEMs) (e.g., for inclusion in products to be distributed to, for example, retailers and/or to other end users such as direct buy customers).

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

In general, the same reference numbers will be used throughout the drawing(s) and accompanying written description to refer to the same or like parts. The figures are not to scale.

### Definitions

Throughout the specification and claims, the following terms take the meanings explicitly associated herein, unless the context clearly dictates otherwise.

As used in this patent, connection references (e.g., attached, coupled, connected, and joined) may include intermediate members between the elements referenced by the connection reference and/or relative movement between those elements unless otherwise indicated. As such, connection references do not necessarily infer that two elements are directly connected and/or in fixed relation to each other. As used herein, stating that any part is in “contact” with another part is defined to mean that there is no intermediate part between the two parts.

When introducing elements of various embodiments of the present disclosure, the articles “a,” “an,” “the,” and “said” are intended to mean that there are one or more of the elements. As used herein, singular references (e.g., “a”, “an”, “first”, “second”, etc.) do not exclude a plurality. The term “a” or “an” object, as used herein, refers to one or more of that object. The terms “a” (or “an”), “one or more”, and “at least one” are used interchangeably herein. Furthermore, although individually listed, a plurality of means, elements or method actions may be implemented by, e.g., the same entity or object. Additionally, although individual features may be included in different examples or claims, these may possibly be combined, and the inclusion in different examples or claims does not imply that a combination of features is not feasible and/or advantageous.

The terms “comprising,” “including,” and “having” are intended to be inclusive and mean that there may be additional elements other than the listed elements. That is, “including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc., may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, or (7) A with B and with C. As used herein in the context of describing structures, components, items, objects and/or things, the phrase “at least one of A and B” is intended to refer to implementations including any of (1) at least one A, (2) at least one B, or (3) at least one A and at least one B. Similarly, as used herein in the context of describing structures, components, items, objects and/or things, the phrase “at least one of A or B” is intended to refer to implementations including any of (1) at least one A, (2) at least one B, or (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, or (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, or (3) at least one A and at least one B.

Unless specifically stated otherwise, descriptors such as “first,” “second,” “third,” etc., are used herein without imputing or otherwise indicating any meaning of priority, physical order, arrangement in a list, and/or ordering in any way, but are merely used as labels and/or arbitrary names to distinguish elements for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for identifying those elements distinctly that might, for example, otherwise share a same name.

As used herein, “approximately” and “about” modify their subjects/values to recognize the potential presence of variations that occur in real world applications. For example, “approximately” and “about” may modify dimensions that may not be exact due to manufacturing tolerances and/or other real world imperfections as will be understood by persons of ordinary skill in the art. For example, “approximately” and “about” may indicate such dimensions may be within a tolerance range of +/−10% unless otherwise specified in the below description. As used herein “substantially real time” refers to occurrence in a near instantaneous manner recognizing there may be real world delays for computing time, transmission, etc. Thus, unless otherwise specified, “substantially real time” refers to real time+/−1 second.

As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

As used herein, the terms “system,” “unit,” “module,” “engine,” etc., may include a hardware and/or software system that operates to perform one or more functions. For example, a module, unit, or system may include a computer processor, controller, and/or other logic-based device that performs operations based on instructions stored on a tangible and non-transitory computer readable storage medium, such as a computer memory. Alternatively, a module, unit, engine, or system may include a hard-wired device that performs operations based on hard-wired logic of the device. Various modules, units, engines, and/or systems shown in the attached figures may represent the hardware that operates based on software or hardwired instructions, the software that directs hardware to perform the operations, or a combination thereof.

As used herein, “processor circuitry” is defined to include (i) one or more special purpose electrical circuits structured to perform specific operation(s) and including one or more semiconductor-based logic devices (e.g., electrical hardware implemented by one or more transistors), and/or (ii) one or more general purpose semiconductor-based electrical circuits programmable with instructions to perform specific operations and including one or more semiconductor-based logic devices (e.g., electrical hardware implemented by one or more transistors). Examples of processor circuitry include programmable microprocessors, Field Programmable Gate Arrays (FPGAs) that may instantiate instructions, Central Processor Units (CPUs), Graphics Processor Units (GPUs), Digital Signal Processors (DSPs), XPUs, or microcontrollers and integrated circuits such as Application Specific Integrated Circuits (ASICs). For example, an XPU may be implemented by a heterogeneous computing system including multiple types of processor circuitry (e.g., one or more FPGAs, one or more CPUs, one or more GPUs, one or more DSPs, etc., and/or a combination thereof) and application programming interface(s) (API(s)) that may assign computing task(s) to whichever one(s) of the multiple types of processor circuitry is/are best suited to execute the computing task(s).

In addition, it should be understood that references to “one embodiment” or “an embodiment” of the present disclosure are not intended to be interpreted as excluding the existence of additional embodiments that also incorporate the recited features.

As used herein, a “toxicity-related model” (also referred to as a toxicity model herein) is a machine learning and/or other artificial intelligence model that processes input data to produce a prediction of toxicity in a patient and/or models another surrogate or substitute characteristic that correlates to a toxicity associated with an immunotherapy (e.g., a risk of harm from a side effect or condition associated with the immunotherapy treatment such as pneumonitis, colitis, hepatitis, etc.). An “efficacy-related model” (also referred to as an efficacy model herein) is a machine learning and/or other artificial intelligence model that processes input data to produce a prediction of efficacy in a patient and/or models another surrogate or substitute characteristic that correlates to an efficacy or effectiveness of an immunotherapy (e.g., a likelihood that the immunotherapy will be effective, such as time-on-treatment, overall survivability, etc.).

A “feature” or feature variable represents an attribute or type of data. A feature can be represented in a variety of formats such as categorical, numerical, a date, percentage, etc.

“Precision” refers to a proportion or percentage of outcomes in which an artificial intelligence model output was actually correct. For example, a model outputs a prediction, and a group of model output predictions can be evaluated to determine a precision of the model. Precision can be evaluated on a scale of 0.0 to 1.0, for example, with 0.0 indicating that the model's prediction was never correct and 1.0 indicating that the model's prediction was always correct. Precision can be defined in terms of true positives (TP) and false positives (FP). True positives were predictions that were correct predictions, and false positives were incorrect predictions. A model's precision can then be determined by: Precision=TP/(TP+FP).

“Recall” refers to a proportion of true positives (actual positives) that were identified correctly. Recall can also be evaluated on a scale of 0.0 to 1.0, for example, with 1.0 indicating that a model never produced a false negative and 0.0 indicating that the model always produced a false negative. Recall can be defined in terms of true positives and false negatives (FN), such that: Recall=TP/(TP+FN).

The term “deep learning” is a machine learning technique that utilizes multiple data processing layers to recognize various structures in data sets and classify the data sets with high accuracy. A deep learning network (DLN), also referred to as a deep neural network (DNN), can be a training network (e.g., a training network model or device) that learns patterns based on a plurality of inputs and outputs. A deep learning network/deep neural network can be a deployed network (e.g., a deployed network model or device) that is generated from the training network and provides an output in response to an input.

The term “supervised learning” is a machine learning training method in which the machine is provided already classified data from human sources. The term “unsupervised learning” is a machine learning training method (e.g., random forest, gradient boosting, etc.) in which the machine is not given already classified data but makes the machine useful for abnormality detection. The term “semi-supervised learning” is a machine learning training method in which the machine is provided a small amount of classified data from human sources compared to a larger amount of unclassified data available to the machine.

The term “convolutional neural networks” or “CNNs” are biologically inspired networks of interconnected data used in deep learning for detection, segmentation, and recognition of pertinent objects and regions in datasets. CNNs evaluate raw data in the form of multiple arrays, breaking the data in a series of stages, examining the data for learned features. Hepatitis and/or toxicity can be predicted using a CNN, for example.

The term “recurrent neural network” or “RNN” relates to a network in which connections between nodes form a directed or undirected graph along a temporal sequence. Hepatitis and/or toxicity can be predicted using a RNN, for example.

The term “transfer learning” is a process of a machine storing the information used in properly or improperly solving one problem to solve another problem of the same or similar nature as the first. Transfer learning may also be known as “inductive learning”. Transfer learning can make use of data from previous tasks, for example.

The term “active learning” is a process of machine learning in which the machine selects a set of examples for which to receive training data, rather than passively receiving examples chosen by an external entity. For example, as a machine learns, the machine can be allowed to select examples that the machine determines will be most helpful for learning, rather than relying only on an external human expert or external system to identify and provide examples.

The term “computer aided detection” or “computer aided diagnosis” refer to computers that analyze medical data to suggest a possible diagnosis.

### Example Model Generation Systems and Associated Methods

A large quantity of health-related data can be collected using a variety of mediums and mechanisms with respect to a patient. However, processing and interpreting the data can be difficult to drive actionable results. For example, understanding and correlating various forms and sources of data through standardization/normalization, aggregation, and analysis can be difficult, if not impossible, given the magnitude of data and the variety of disparate systems, formats, etc. As such, certain examples provide apparatus, systems, associated models, and methods to process and correlate health-related data to predict patient outcomes and drive patient diagnosis and treatment. Certain examples provide systems and methods for health data predictive model building. Certain examples provide a framework and machine learning workflow for therapeutic prediction.

For example, immune checkpoints regulate the human immune system. Immune checkpoints are pathways that allow a body to be self-tolerant by preventing the immune system from attacking cells indiscriminately. However, some cancers can protect themselves from attack by stimulating immune checkpoints (e.g., proteins on immune cells). To target cancer cells in the body, Immune Checkpoint Inhibitors (ICIs) can be used to target these immune checkpoint proteins to better identify and attack, rather than hide, cancerous cells.

Despite the great success of ICI cancer treatments, such treatments can pose a great threat to human health, due to their side effect, which is a type of immune-related Adverse Events (irAE) caused by these treatment options. One of these toxicities is hepatitis, which occurs when the liver is affected by the auto-immune-like inflammatory pathological process triggered by ICI. Certain examples predict the onset of irAE hepatitis before the start of the first ICI treatment. More precisely, certain examples predict whether irAE hepatitis will happen in a given time-window after the initiation of the first treatment. Other toxicities such as pneumonitis, colitis, etc., can be similarly predicted.

For example, majority class undersampling is combined with time series data aggregation to obtain a well-balanced and static dataset, which can be fed to the models. Example models include Gradient Boosting (GB) and Random Forest (RF), and/or other models able to accommodate the size and statistical properties of the data. The model can be selected based on a model selection criterion such as an F1 score, which is a measure of the model's accuracy on a dataset. For example, a GB model without undersampling can maximize an F1-score (e.g., harmonic mean of recall and precision), and a RF model with undersampling can provide a high recall (e.g., ratio of true positives found) with relatively low precision (e.g., ratio of true positives among the estimates), which is acceptable due to the cost effectiveness of additional tests required based on the decision of the model. The models are also able to create probability estimates for a label, rather than only the discrete labels themselves.

Input data is prepared to develop and/or feed model(s) to drive prediction, therapy, etc. In certain examples, input is prepared by extracting blood feature information (e.g., a relevant section of blood features, etc.) from Electronic Health Record (EHR) data tables (e.g., received at a processor/processing circuitry from the EHR, etc.), electronic medical record (EMR) information, etc. The blood features are measurements of liver biomarker concentration in blood plasma (such as ALT, AST, Alkaline Phosphatase and Bilirubin, etc.) and other concentration values in the blood. Blood features can be represented as time series data, for example. After blood features are extracted, the time series data is formed into a single complex data structure. The data structure is used to aggregate time series blood feature data into a data table, which can be used with preprocessing and transformation.

For example, feature engineering aggregates the blood feature data by describing the time-series data of the blood particles with an associated mean, standard deviation, minimum, and maximum. Liver features can also be created by taking the last liver biomarker measurements available before treatment. Labels can be created (e.g., using a definition obtained from medical experts, etc.) to classify someone as positive when a level of at least one liver biomarker exceeds a threshold (e.g., 3-times the upper limit of normal, etc.) within a predefined window. Otherwise, a label can classify the patient as negative. A date of an immune checkpoint inhibitor (ICI) treatment can be determined and/or otherwise provided for use with the label and/or the time-series.

Other features can include lung-related code (e.g., ICD-9, ICD-10, etc.) frequency, frequency of C34 code, frequency of C78 code, smoking, oxygen saturation in blood, frequency of blood hemoglobin below normal, frequency of blood hemoglobin above normal, albumin blood frequency below normal, albumin blood frequency above normal, frequency of white blood cell count below normal, frequency of white blood cell count above normal, frequency of red blood cell count below normal, frequency of red blood cell count above normal, frequency of lymph node readings above normal, lymph frequency below normal, liver enzymes, drug class, interaction, maximum, minimum, time weighted average, last value, days before first treatment, etc.

After the input data is prepared (e.g., using feature engineering), the dataset is resampled. That is, the dataset resulting from the input preparation is unbalanced. As such, the dataset can be processed to infer, validate, estimate, and/or otherwise resample the prepared feature data in the dataset. For example, random majority class undersampling is performed on the dataset when the goal is to maximize the recall value. When the F1-score is the subject of maximization, then the resampling can be skipped or disregarded.

Using the dataset (e.g., resampled or otherwise), a model can be trained and tested to generate a prediction. For example, when recall maximization is desired as the model selection criterion, the dataset can be used to train an RF model. When F1-score maximization is desired as the model selection criterion, the dataset can be used to train a GB model, for example. As such, the model selection criterion can be used to determine a desired goal, target, or focus, such as recall maximization, F1-score maximization, precision maximization, etc. For example, a model generated to configure a cohort of patients for a clinical trial may be selected or otherwise set to admit many patients into the trial. As another example, a model generated to determine a treatment plan for a patient can be selected to proceed cautiously (or alternatively, aggressively) with treatment and associated risk based on a likelihood of toxicity balanced with a likelihood of efficacy. For example, a high risk of toxicity combined with a low chance of efficacy may keep a patient from an immunotherapy treatment, while a low risk of toxicity paired with a chance of efficacy encourages the patient to begin (or continue) the immunotherapy treatment. A relatively even balance of toxicity risk and efficacy probability can leave selection to the interests, preferences, and/or objectives of the patient and/or their physician, for example. Additionally, safety monitoring, blood tests, and/or other measures (e.g., periodic reevaluation, check-in, etc.) may be ordered depending on the likelihood of toxicity, in order to detect any toxicities early and be able to manage them while continuing treatment. In certain examples, the trained model can be validated, such as with Leave-One-Out Cross-Validation, where each sample is predicted individually with the rest as the training set.

As such, a variety of “artificial intelligence” (AI) models can be developed and deployed for use in a variety of health prediction applications. For example, a model can be used to predict static and/or dynamic prognostic factors for hepatitis using an AI model and patient (e.g., EHR, etc.) data.

Alternatively or additionally, a predictive model can be developed for ICI-related pneumonitis using small, noisy datasets. Using input data from structured (e.g., EHR, EMR, laboratory data system(s), etc.) and/or unstructured (e.g., curated from laboratory data system(s), EHR, EMR, etc.) data, input features can be evaluated to build models and output a predicted probability of developing pneumonitis. In certain examples, multiple models can be developed, and the system and associated process can iterate and decide between two or more model versions. For example, available data can be divided into two partitions with a sequential forward selection process, and robust performance evaluation can be used to validate and compare two developed models to select one for deployment.

For example, available data can be divided into two partitions, and a sequential forward selection process can be utilized to build a model. In each iteration, two model versions (e.g., two adjusted models refined from an initial model, etc.) are compared, and the one with higher performance on both partitions is selected. The second model version (e.g., an adjusted model) is created by adding a candidate feature to the first model version (e.g., the initial model or a prior adjusted model). On the larger partition, the comparison is based on performance results in the inner loop of a nested cross-validation (CV). On the smaller partition, permutation test is performed to test whether the candidate feature has predictive power (e.g., the second model version has higher performance). At the end, the outer loop of nested CV is used for robust performance evaluation of the final model.

Certain examples provide an automated framework to prepare EHR and/or other health data for use in machine learning-based model training. For example, the framework prepares data from multiple sources and generates combined time-dependent unrestricted intermediary outputs, which are used to aggregate features for training and deployment of time-dependent models. For example, input data sources can be processed, and the data is used to generate patient vectors. The patient vectors can be used to filter and aggregate, forming an interface definition. As such, a model-agnostic workflow creates input datasets for multiple model training. Intermediary outputs retain temporal structure for sequential modeling tasks and form a maintainable, sustainable framework with interface.

Certain examples provide predictive model building related to ICI, in which input data from multiple sources is prepared. Ground truth prediction labels can be generated from the prepared data and/or labels can be expertly created. Then one or more models are built on a feature matrix generated using labels and data, with ground truth prediction labels as standalone module of the framework. The framework can then drive a workflow to assess multiple efficacy surrogate endpoints to predict response(s) to ICI therapies, for example.

In certain examples, patient health data is prepared and used to train a model using a system involving a plurality of submodules. For example, the system includes a data extraction and processing submodule to extract patient blood test histories from EMR/EHR, clean the blood history data, and perform data quality check(s). A label definition submodule defines one or more feature labels related to the blood history data, and a feature engineering submodule can form blood features by aggregating and processing blood history data with respect to the labels. A model submodule trains and evaluates an AI model to dynamically predict immune-related hepatitis adverse event risk from fixed length blood test histories. Alternatively or additionally, liver function test values can be extracted, cleaned, and organized in a time series. A label definition algorithm can be executed to generate an AI model and target label for each set of blood and/or liver test values, while feature engineering (e.g., normalization, symbolic transformation and motif extraction) can be used train and evaluate AI risk prediction model(s), for example. Similarly, drug history information, medical condition history, anthropometric features, etc., can be used for labeling and feature formation.

Because features can vary significantly between toxicities, toxicity-specific feature formation and model training is important to provide meaningful features and accurate predictive results. Otherwise, a lack of meaningful features destroys performance of the resulting model. Thus, certain examples derive models focused on particular toxicities, and the outputs can be combined (together and/or further with a prediction of efficacy) to form a recommendation, such as based on a risk versus benefit analysis of toxicities versus efficacy for a given immunotherapy drug.

As such, certain examples drive therapy based on a prediction of the likelihood of complications from hepatitis, pneumonitis, etc. Patients can be selected for immunotherapy treatment, be removed from immunotherapy treatment and/or otherwise have their treatment plan adjusted, be selected for an immunotherapy clinical trial, etc., based on a prediction and/or other output of one or more AI models. Model(s) used in the prediction can evolve as data continues to be gathered from one or more patients, and associated prediction(s) can change based on gathered data as well. Model(s) and/or associated prediction(s) can be tailored to an individual and/or deployed for a group/type/etc. of patients, or for a group or individual ICI drug, etc., for example.

In certain examples, data values are normalized to an upper limit of a “normal” range (e.g., for blood test, liver test, etc.) such that values from different sources can be compared on the same scale. Data values and associated normalization/other processing can be specific to a lab, a patient, a patient type (e.g., male/female, etc.), etc. For example, each lab measurement may have a specific normal range that is used to evaluate its values across multiple patients.

With time-series data, one value depends on a previous and/or other value such that the data values have a relationship, rather than being independent. The dependency can be identified and taken into account to identify patients in the data over time. For example, if a data value in a times series of patient blood work exceeds twice a normal limit at a first time, reduces within the normal limit at a second time, and again exceeds twice the normal limit at a third time, then this pattern can be identified as important (e.g., worth further analysis). Data processing can flag or label this pattern accordingly, for example. As such, clinical data from a patient's record can be used over time to identify and form features, anomalies, other patterns, etc. Data is confused to a common model for comparison. Resulting models trained and tested on such data can be robust against outliers, scaling, etc. Features can be created for better (e.g., more efficient, more accurate, more robust, etc.) modeling such as pneumonitis modeling, colitis modeling, hepatitis modeling, etc.

Thus, data processing creates feature(s) that can be used to develop model(s) that can be deployed to predict outcome(s) with respect to patient(s). For example, a data processing pipeline creates tens of thousands of features (e.g., pneumonitis, frequency of ICD-10 codes such as frequency of C34 codes, etc.).

For example, data values can include ICD-10 codes for a given patient for a one-year time period. In certain examples, codes can span multiple years (e.g., a decade, etc.) and be harmonized for processing. The ICD-10 codes are processed to identify codes relevant to lung or respiratory function, and such codes can then be used to calculate a relative frequency of lung disease in the patient. As another example, a patient history can be analyzed to determine a relative frequency of a C34 code in the patient history, which is indicative of lung cancer. Smoking status can also be a binary flag set or unset from the data processing pipeline, for example. In certain examples, codes can be converted between code systems (e.g., ICD-9, ICD-10 (such as C34, C78, etc.), etc.). Codes can be reverse-engineered without all of the keys, for example.

Using codes and other data, a plurality (e.g., 5, 6, 10, etc.) of features can be created and used in a modeling framework to predict development of pneumonitis in a patient. The model is built in a stepwise, forward fashion. Labels for pneumonitis models are not inherently in the dataset, so a ground truth is created for model training based on expert judgment to identify labels from patient history (-ies), for example. Codebooks and quality control can be used to correctly label, for example.

In certain examples, historical data received from patients is asynchronous. Systems and methods then align the data for the patient (and/or between patients) to enable aggregation and analysis of the data with respect to a common baseline or benchmark. In certain examples, an influence point, anchor point, or other reference can be selected/determined, and patient data time series/timelines are aligned around that determined or selected point (e.g., an event occurring for the patient(s) such as a check-up, an injury, an episode, a test, a birthdate, a milestone, etc.). For example, a date of first chemotherapy, ICI therapy, first symptom/reaction (e.g., in lung function, etc.), etc., can be used to align patient data.

Processed data can then be used to predict static labels in a predefined or otherwise determined time window, etc. Models can be trained, validated, and deployed for hepatitis, pneumonitis, drug efficacy, etc. As such, data from an EHR, EMR, laboratory system, and/or other data source can be pre-processed and provided to a model to generate a prediction, which can be post-processed and output a user and/or other system for alert, follow-up, treatment protocol, etc. In certain examples, the prediction value is routed to another system (e.g., scheduling, lab, etc.) for further processing.

One or more AI models can be used to facilitate processing, correlation, and prediction based on available patient health data such as blood test results, liver test results, other test results, other patient physiological data, etc. Models can include a high recall low precision model, a low recall high precision model, a harmonic mean maximized (convergence) model, etc. A boosted decision tree model or variant such as random forest (RF), gradient boosting (GB), etc., can be used. For example, a majority class undersampling, random forest model can be used to maximize recall with relatively low precision. However, with hepatitis, prevention is inexpensive and easy, so false negatives can be afforded. As such, a gradient boosting model can be developed to maximize F1 score with no resampling applied.

Machine learning techniques, whether deep learning networks or other experiential/observational learning system, can be used to characterize and otherwise interpret, extrapolate, conclude, and/or complete acquired medical data from a patient, for example. Deep learning is a subset of machine learning that uses a set of algorithms to model high-level abstractions in data using a deep graph with multiple processing layers including linear and non-linear transformations. While many machine learning systems are seeded with initial features and/or network weights to be modified through learning and updating of the machine learning network, a deep learning network trains itself to identify “good” (e.g., useful, etc.) features for analysis. Using a multilayered architecture, machines employing deep learning techniques can process raw data better than machines using conventional machine learning techniques. Examining data for groups of highly correlated values or distinctive themes is facilitated using different layers of evaluation or abstraction.

Deep learning is a class of machine learning techniques employing representation learning methods that allows a machine to be given raw data and determine the representations needed for data classification. Deep learning ascertains structure in data sets using backpropagation algorithms which are used to alter internal parameters (e.g., node weights) of the deep learning machine. Deep learning machines can utilize a variety of multilayer architectures and algorithms. While machine learning, for example, involves an identification of features to be used in training the network, deep learning processes raw data to identify features of interest without the external identification.

Deep learning in a neural network environment includes numerous interconnected nodes referred to as neurons. Input neurons, activated from an outside source, activate other neurons based on connections to those other neurons which are governed by the machine parameters. A neural network behaves in a certain manner based on its own parameters. Learning refines the machine parameters, and, by extension, the connections between neurons in the network, such that the neural network behaves in a desired manner.

A variety of artificial intelligence networks can be deployed to process input data. For example, deep learning that utilizes a convolutional neural network segments data using convolutional filters to locate and identify learned, observable features in the data. Each filter or layer of the CNN architecture transforms the input data to increase the selectivity and invariance of the data. This abstraction of the data allows the machine to focus on the features in the data it is attempting to classify and ignore irrelevant background information.

Deep learning operates on the understanding that many datasets include high level features which include low level features. While examining an image, for example, rather than looking for an object, it is more efficient to look for edges which form motifs which form parts, which form the object being sought. These hierarchies of features can be found in many different forms of data such as speech and text, etc.

Learned observable features include objects and quantifiable regularities learned by the machine during supervised learning. A machine provided with a large set of well classified data is better equipped to distinguish and extract the features pertinent to successful classification of new data.

A deep learning machine that utilizes transfer learning may properly connect data features to certain classifications affirmed by a human expert. Conversely, the same machine can, when informed of an incorrect classification by a human expert, update the parameters for classification. Settings and/or other configuration information, for example, can be guided by learned use of settings and/or other configuration information, and, as a system is used more (e.g., repeatedly and/or by multiple users), a number of variations and/or other possibilities for settings and/or other configuration information can be reduced for a given situation.

An example deep learning neural network can be trained on a set of expert classified data, for example. This set of data builds the first parameters for the neural network, and this would be the stage of supervised learning. During the stage of supervised learning, the neural network can be tested to determine whether the desired behavior has been achieved.

Once a desired neural network behavior has been achieved (e.g., a machine has been trained to operate according to a specified threshold, etc.), the machine can be deployed for use (e.g., testing the machine with new/updated data, etc.). During operation, neural network classifications can be confirmed or denied (e.g., by an expert user, expert system, reference database, etc.) to continue to improve neural network behavior. The example neural network is then in a state of transfer learning, as parameters for classification that determine neural network behavior are updated based on ongoing interactions. In certain examples, the neural network can provide direct feedback to another process. In certain examples, the neural network outputs data that is buffered (e.g., via the cloud, etc.) and validated before it is provided to another process.

Deep learning machines can utilize transfer learning when interacting with physicians to counteract the small dataset available in the supervised training. These deep learning machines can improve their computer aided diagnosis over time through training and transfer learning. However, a larger dataset results in a more accurate, more robust deployed deep neural network model that can be applied to transform disparate medical data into actionable results (e.g., system configuration/settings, computer-aided diagnosis results, image enhancement, etc.).

One or more such models/machines can be developed and/or deployed with respect to prepared and/or curated data. For example, features can be extracted from EHR/EMR data tables (e.g., liver biomarkers, blood/plasma concentration, etc.), etc. Curated data extracts structured data from unstructured sources (e.g., diagnosis dates from medical notes, etc.), for example. Extracted features form the basis of label creation. Time series measurements are extracted and aggregation produces statistical descriptors for the time series data. A table of such data is then used to train a model to make predictions. In some examples, the data can be resampled if necessary, desired, or determined. The models are validated using a robust cross-validation, such as leave-one-out cross validation.

For example, a selection, input, or target can determine whether to maximize an F1 score or recall with a given model. When F1 score is to be maximized by the model, then no resampling of the data is performed. When recall is to be maximized by the model, then resampling of the data can be performed. The decision can be driven by the deployment environment for the model, for example. When the model is to be deployed in a system facilitating a drug trial, then a F1 score is to be maximized because the drug company wants patients with a highest chance to respond well to a given drug. High recall is more important in a clinical treatment setup where the system wants to eliminate as many toxicities as possible. In a treatment setup for hepatitis, for example, low model precision is acceptable due to the inexpensive nature of treatment for hepatitis.

Associated systems and methods can be used to assess a probability or reliability of a prediction generated by a model. The prediction and associated confidence level or other reliability can be output to another processing system, for example. Probable reactions to immunotherapy treatments can be modeled based on available patient data collected in clinical practice.

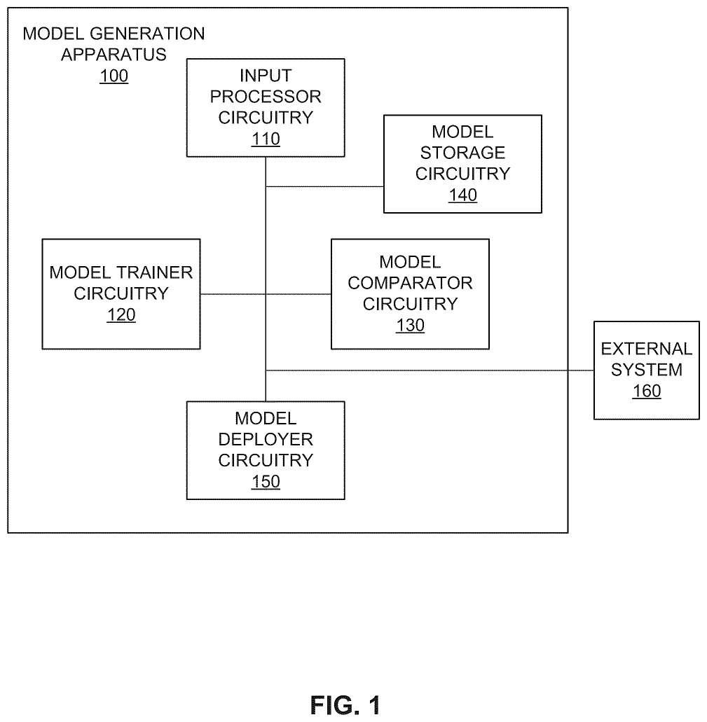

FIG. 1 illustrates an example model generation apparatus 100 including example input processor circuitry 110, example model trainer circuitry 120, example model comparator circuitry 130, example model storage circuitry 140, and example model deployer circuitry 150. As shown in the example of FIG. 1 the example input processor circuitry 110 processes input data pulled from a record to form a set of candidate features. The example model trainer circuitry 120 trains at least a first model and a second model using the set of candidate features. The example model comparator circuitry 130 tests at least the first model and the second model to compare performance of the first model and the second model. Based on the comparison, the example model comparator circuitry 130 selects at least one of the first model or the second model based on the comparison. The example model storage circuitry 140 provides memory circuitry to store the selected first and/or second model. The example model deployer circuitry 150 deploys the selected first model and/or second model to predict a likelihood of a toxicity, such as pneumonitis, hepatitis, colitis, etc., occurring due to immunotherapy according to a treatment plan for a patient.

In operation, the example input processor circuitry 110 processes input data related to one or more patients such as laboratory results, diagnosis codes, billing codes, etc. Input can originate at one or more external systems 160 such as an EHR, EMR, etc. The example input processor circuitry 110 can extract and organize the input in a time series for each patient, for example. In certain examples, the input processor circuitry 110 aligns the input data with respect to an anchor point to organize the input data in the time series. The anchor point is a reference or alignment point in time for the input data, such as a patient start to immunotherapy treatment, a first patient symptom, etc., that enables a group of patients to be tracked, modeled, and compared with respect to that reference point in time, for example. In certain examples, the input processor circuitry 110 generates labels for the input data to form the set of candidate features.

In certain examples, the input processor circuitry 110 performs feature engineering on the set of candidate features and/or underlying data. For example, the input processor circuitry feature engineers the set of candidate features by normalizing, transforming, and/or extracting, for example, from the set of candidate features. In certain examples, the input processor circuitry 110 selects from the set of candidate features to form a patient feature set to train and/or test at least the first model and the second model based on the feature engineering. In certain examples, the input processor circuitry 110 generates a feature matrix to train and/or test/validate the first model and/or the second model based on the feature engineering.

In certain examples, the model deployer circuitry 150 deploys the selected first and/or second model as an executable tool with an interface to facilitate gathering of patient data and interaction with the selected first and/or second model. The deployed model(s) can be used with the tool to select a patient for clinical trial, commence a course of immunotherapy treatment according to a treatment plan or protocol, adjust a current immunotherapy treatment plan, etc. In certain examples, the deployed model(s) can be stored and/or utilized in one more external systems 160 such as EHR, EMR, scheduling system, clinical information system, etc. The tool, implemented as a portal, an online platform, a computer application, etc., can be used to load one or more stored models (e.g., a toxicity-related model, an efficacy-related model, etc.), determine one or more thresholds or other patient selection criterion by which to evaluate an output of the model(s), process healthcare data for a given patient using the model(s), and evaluating the predictive output(s) of the model(s) with respect to the threshold(s) to take an action with respect to the patient (e.g., configure a treatment plan for the patient to include or exclude an immunotherapy, where that treatment plan can be a treatment plan for a cohort of patients in a clinical trial, etc.) based on the threshold.

As such, the example model generator apparatus 100 can pre-process data from one or more external systems 160 via the example input processor circuitry 110. The example model generator apparatus 100 then trains and validates a plurality of models using the model trainer circuitry 120 and the model comparator circuitry 130. The example model generator apparatus 100 post-processes, stores, and deploys one or more of the trained, validated models using the model storage circuitry 140 and the model deployer circuitry 150. Input data from an EHR, EMR, and/or other external system 160 is transformed into a plurality of models that can be stored and deployed to predict treatment efficacy, risk of toxicity and/or other side effect, etc., from immunotherapy treatment of a particular patient. Data from a plurality of patients can be leveraged through processing and modeling to generate targeted prediction for a particular patient, for example.

In certain examples, the model trainer circuitry 120 trains and validates a boosted decision tree model, other ensemble and/or regression-based learning model, etc. For example, a boosted decision tree model leverages a plurality of weak-learning decision trees to create a strong learning AI model. Trees in the model can correct for errors in other trees of the model, for example, and the entire ensemble of trees in the model works together to generate the predictive output. Models can also be formed as random forest (RF) models, gradient boosted (GB) models, etc. Data is conformed by the model trainer circuitry 120 to a common model for comparison that is robust against outliers and able to be scaled, for example.

In certain examples, the input data processor 110 normalizes input data values to an upper limit of normal such that values (e.g., blood work values, liver function test values, other lab work values, etc.) are on the same scale. Each lab value may have a certain range of values. The input data processor 110 normalizes values for a particular lab type over a range so that the data for that lab is comparable across patients, for example. The input data processor 110 can also organize data into a time series. The time series data can be normalized and/or otherwise adjusted by the input data processor 110 based on an anchor point (e.g., a common or reference event such as hospitalization, symptom, birth, date of diagnosis, date of first ICI administration, etc., around which data and associated feature(s) can be aligned for model development and prediction).

For example, received patient data may be asynchronous. Selecting an anchor/inflection/influence point allows the data to be aligned for same, similar, and/or different patients. For example, in a patient group with diabetes, ten years of patient history is reviewed while excluding data occurring after diagnosis. The point of diagnosis is selected as an anchor point for a retrospective look at the patient history data (e.g., when no blood sugar levels were being obtained or insulin taken, etc.). As another example, a data of first immunotherapy can be used to align patient data. Aligning a plurality of patients with respect to their start of immunotherapy allows correlation between subsequent events for patients in the plurality of patients, for example.

For example, date of birth, date of first immunotherapy, date of last immunotherapy, and date of death (or knowledge that the patient was still alive five years after start of immunotherapy), can be used to align all patients to the start of their immunotherapy as the anchor point, regardless of how old the patients were at that point in time. As such, the data is organized for identification of relationships and comparisons, for example. Patterns can be identified in the data, for example.

In certain examples, the time series data is formed by the input data processor 110 into a plurality of features. These features can be a set of candidate features from which features are selected to train and validate the models. For example, patient demographic data, biometric data, examination data, laboratory data, and other patient healthcare data can be extracted from patient records to form an input data set that can be associated with a plurality of features, such as red blood cell value, white blood cell value, drug count for one or more drugs, blood albumin, lymph system, alkaline phosphate, radiology, etc.

Feature engineering by the input data processor 110 can form a plurality of features based on codes (e.g., ICD-10 codes, etc.), for example. For example, ICD-10 codes for a patient in a given year can be processed to identify codes relevant to lung and/or respiratory function (C34, C78, etc.), and the time series of those codes can form a function used to calculate a relative likelihood of lung disease in the patient. Similarly, a plurality of features can be formed to predict development of pneumonitis, hepatitis, colitis, and/or other toxicity from immunotherapy treatment.

Features categorizing or identifying data based on lung function, liver biomarkers, blood work (e.g., concentration in blood, plasma, etc.), etc., can form the basis for label and/or other statistical descriptor creation for model training. For example, the gathered data can be compared to a ground truth set of identified labels to generate labels for the data. Resampling can be conducted as well as a validation (e.g., leave-one-out cross validation, other cross-validation, other validation, etc.) to generate a robust model.

The example model trainer circuitry 120 uses the features and/or other data to train one or more AI models, such as a toxicity prediction model (e.g., a hepatitis prediction model, a colitis prediction model, a pneumonitis prediction model, etc.), an efficacy prediction model (e.g., immunotherapy efficacy model, etc.), etc. The models can be high recall with low precision, low recall with high precision, harmonic mean (F1) maximized, majority claim undersampling, etc. A determination of model focus can be made by and/or for the example model trainer circuitry 120 (e.g., based on a setting, mode, type, other model selection criterion, etc.). For example, the model trainer circuitry 120 trains one or more models to maximize the F1 score or to maximize recall. When focusing on F1, resampling may not be necessary. When focusing on recall, resampling (e.g., with a training data set and a test data set, multiple training data sets and/or multiple test data sets, etc.) may be desirable to improve the model.

For example, when configuring participation in a drug trial, a focus is on F1 score maximization to identify patients with a highest chance to respond well to a given immunotherapy drug. When determining a clinical treatment for a patient, however, a goal is to eliminate as many toxicities as possible for the patient. As such, a model developed with high recall is more important, and low precision is acceptable because of inexpensive treatment options for hepatitis, colitis, pneumonitis, etc.

The model comparator circuitry 130 can compare and/or otherwise assess multiple trained models to validate the models and evaluate the probably of the prediction generated by each model. Such assessment/evaluation can be used to select one or more models to be deployed. For example, models can be compared and/or otherwise evaluated based on mean output values, standard deviation of model output, etc. One or more models can be selected for storage in the model storage circuitry 140 and/or other memory circuitry, deployment in a tool via the model deployer circuitry 150, output to one or more external systems 160, etc.

In certain examples, an external system 160 can leverage one or more deployed models from the model deployer circuitry 150 to drive a tool to evaluate efficacy and/or toxicity associated with immunotherapy for a patient and/or patient population, etc. Using one more deployed models, a patient can be assessed at the start of an immunotherapy treatment plan, during administration of the immunotherapy treatment plan, as a candidate for an immunotherapy clinical trial, etc. In certain examples, prior efficacy and/or toxicity model predictions for the patient can be used with updated efficacy and/or toxicity model predictions for the patient (e.g., in combination between prior and current results, with prior as an input to produce current results, etc.).

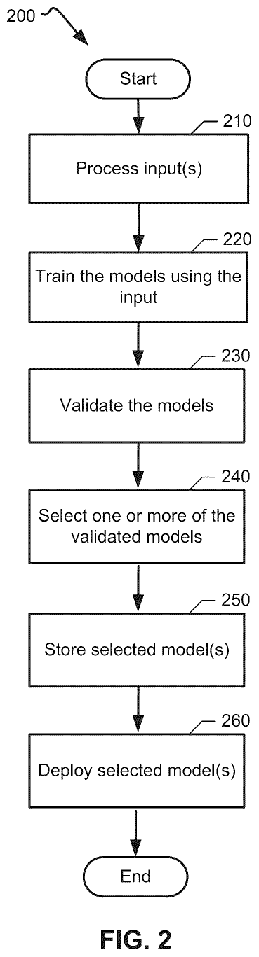

FIGS. 2-14 are flow charts of example processes representing computer-readable instructions storable in memory circuitry and executable by processor circuitry to implement and actuate the example model generation apparatus 100 of FIG. 1. The example process 200 of FIG. 2 begins at block 210, at which the input processor circuitry 110 processes data to form input(s) for the model trainer circuitry 120. For example, the input processor circuitry 110 can align data with respect to an anchor or reference point (e.g., a date, an event, a milestone or other marker, etc.). The input processor circuitry 110 can form a time series from the data. Alternatively or additionally, the input processor circuitry 110 can label and/or perform feature engineering on the data to form an input feature set to train the model(s), for example.

At block 220, models are trained using the input. For example, a plurality of AI models are trained by the model trainer circuitry 120 using the input feature set and/or other input provided from the input processor circuitry 110 with associated data. By identifying patterns and correlations in the input data based on the features, the model trainer circuitry 120 forms and weights nodes, layers, connections, etc., in the models (e.g., a RF model, a GB model, a boosted decision tree model, etc.), for example. Models can be tuned by iteratively adding and removing features to find a balance of precision and recall in the model, for example.

At block 230, the trained models are validated by the model trainer circuitry 120. For example, additional input (e.g., features, resampled input data, etc.) is used to validate (e.g., test, verify, etc.) that the trained models work as intended with a threshold level of precision, accuracy, etc.

At block 240, the model comparator circuitry 130 evaluates the validated models to select one or more of the models for deployment. For example, the model comparator circuitry 130 can compare and/or otherwise assess multiple trained, validated (e.g., tested, etc.) models according to one or more model selection criterion to evaluate the probably of the prediction generated by each model, the suitability of each model for a particular purpose (e.g., prediction of immunotherapy treatment efficacy, prediction of immunotherapy toxicity, immunotherapy clinical trial selection, immunotherapy treatment plan adjustment, etc.). Such assessment/evaluation can be used by the model comparator circuitry 130 to select one or more models. For example, models can be compared and/or otherwise evaluated based on mean output values, standard deviation of model output, etc. In certain examples, the model comparator circuitry 130 selects one model for use/deployment. In other examples, the model comparator circuitry 130 selects a plurality of models (e.g., a toxicity prediction model and an efficacy prediction model, etc.) for use/deployment.

At block 250, the one or more selected models can be stored by the model storage circuitry 140. As such, the selected model(s) can be saved for later use, deployment, etc. In some examples, unselected model(s) can also be stored by the model storage circuitry 140, as the unselected model(s) may be suitable for other usage/deployment in response to a subsequent request.

At block 260, the one or more selected models are deployed by the model deployer circuitry 150 to the external system 160. The model(s) can be deployed as part of a tool for immunotherapy treatment and/or clinical trial planning to provide a toxicity prediction, an efficacy prediction, etc.

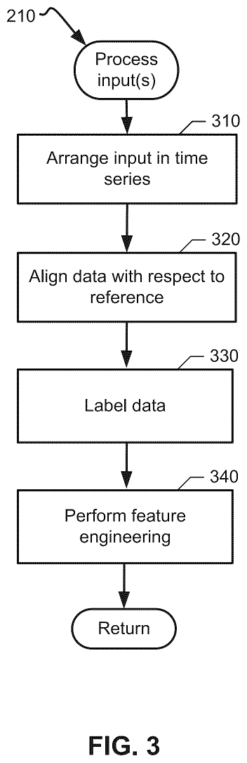

FIG. 3 illustrates an example implementation of input processing for model development (e.g., block 210 of the example of FIG. 2). At block 310, input data for each of a plurality of patients is arranged in one or more time series (e.g., put in order of time to show evolution, progression, dependency, and/or other pattern). At block 320, the time series data for each of the plurality of patients is aligned with respect to an anchor point or other reference event. For example, the various sets of time series data can be aligned according to birth, onset of a condition, event such as hospitalization, etc. By aligning the time series, the data in the time series can be more meaningfully evaluated, compared, and/or otherwise analyzed, for example.

At block 330, the aligned time series data is labeled. For example, target labels and associated grades can be generated for each set of data values (e.g., blood test values, liver function values, lung function values, etc.).

At block 340, feature engineering is performed on the data to prepare the data as a set of features with labels to be used to train, test, and/or otherwise validate models. For example, feature engineering can normalize, transform, and extract the data into a set of patient features for application to a model. In certain examples, the features form a set of candidate features that is evaluated to select a certain subset of patient features for model training, validation, etc.

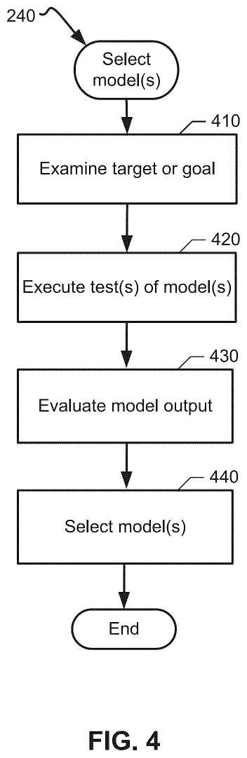

FIG. 4 illustrates an example implementation of model selection for deployment (e.g., block 240 of the example of FIG. 2). At block 410, a target or goal is examined. For example, a request for AI model may be for clinical trial evaluation, immunotherapy efficacy prediction, immunotherapy-associated toxicity and/or other side effect prediction, etc. Such a request can drive selection of a model with a certain characteristic such as high F1 score, high recall, etc. Based on the requested target or goal, a certain subset of available models can be selected. Put another way, based on the requested target or goal, a certain subset of available models can be excluded, leaving a subset of models available for further evaluation and selection.

Further, a target or goal can be associated with a risk or tolerance. For example, a clinical trial may tolerate low efficacy but be interested in low toxicity as well. As another example, a clinical trial may only be concerned with a risk of toxicity associated with a given immunotherapy as a gatekeeper to including or excluding patients from the clinical trial. Alternatively, a selection criterion for patient treatment using a given immunotherapy can balance a risk of toxicity and likely efficacy to determine whether a patient should receive (or continue to receive) the treatment, for example.

At block 420, a test is executed using each remaining model. For example, a permutation test, binomial test, and/or other comparative operation is executed using each model (e.g., evaluating lung function models with respect to smoking, no smoking, other binomial and/or permutation test, etc.). At block 430, an output of each model executing the test is evaluated based on one or more criterion. For example, each model can be evaluated based on mean, standard, and/or other comparative criterion to determine which model best satisfies the one or more criterion.

At block 440, based on the comparison of output and/or other model performance, one or more models are selected. For example, a single model may be selected for deployment based on the evaluation of how the model performed and/or output with respect to the one or more criterion. In some examples, multiple models may be selected based on the one or more criterion and/or other factor. For example, a plurality of models may satisfy the criterion (-ia). As another example, a request may include a request for treatment efficacy as well as likelihood of associated toxicity. In response to such a request, both an efficacy model and a toxicity model can be selected. In some examples, a single model may be trained on both toxicity and efficacy to output a combined prediction.

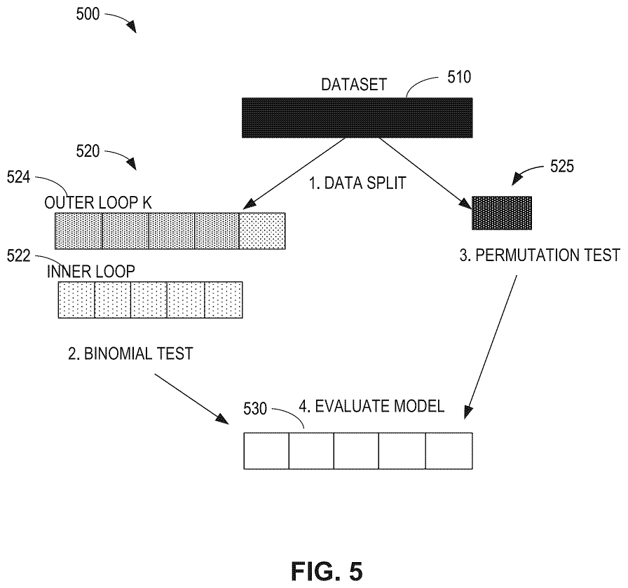

FIG. 5 illustrates an example sequential procedure 500 for model building and evaluation (e.g., through development of a feature set, etc.). As shown in the example of FIG. 5, at 1, an initial data set 510 is split into at least two partitions. Separate data analysis is performed on a first partition of the data 520 and a second partition of the data 525. The first data partition 520 is divided into at least two loops, such as an inner loop 522 and an outer loop 524 for data analysis. At 2, a first test, such as a binomial test evaluates data values in the inner loop 522 and the outer loop 524 (e.g., reject if a data value H₀ includes “smoking”, etc.) to train a model. At 3, the separate, “no touch” test data set 525 is processed using a permutation test, rather than using separate loops. The data set 525 can be processed over multiple permutations with a criterion such as with smoking versus without smoking to identify a likelihood of smoking greater than without smoking, for example. At 4, the model is evaluated to report mean, standard, and/or other evaluation/performance statistic with respect to the first and second data partitions 520, 525 and associated tests.

In certain examples, one or more of the loops 522, 524 are used to evaluate the first data partition 520 via exploratory data analysis with a K-fold cross-validation (CV) comparing a first feature set M1 and a second feature set M2. Through sequential iteration, a feature F is added to the first feature set M1 if K-fold CV performance of M1 is less than M2. Once features have been evaluated accordingly, the resulting model performance is tested using the second data partition 525. If both loops 522, 524 are utilized, M1 and M2 can be compared based on an inner loop 522 result (e.g., a binomial test). When M2 is chosen, a permutation test is performed to assess whether M2 is sufficiently better than M1 on the validation data set 525. Then, model performance can be evaluated on the outer loop 524, for example. As such, an initial feature set, M1, or an extended feature set, M2, can be selected for model training.

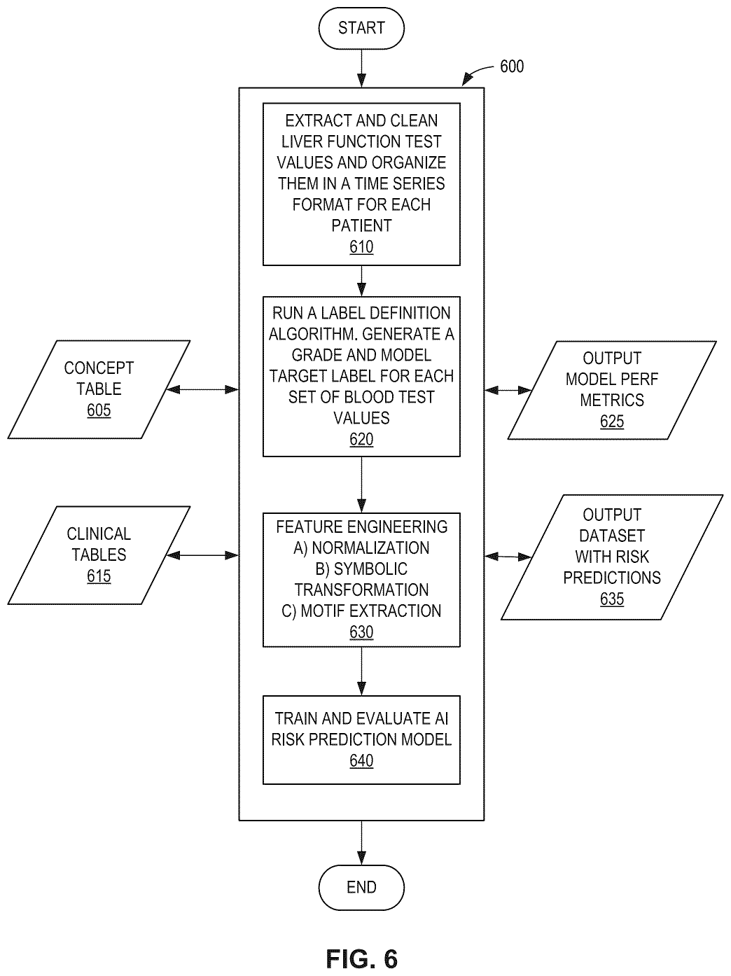

FIG. 6 illustrates an example process 600 for preparing input data for model training. Using the example process 600, data from disparate observational databases can be transformed into a common format (e.g., data model) and common representation (e.g., common terminology, vocabulary, coding scheme, etc.) for systematic analysis according to a library of analytic routines. The example process 600 accepts an input of concept table(s) 605 including concept identifiers, concept names, concept descriptions, etc. For example, concepts in this example relate to liver function tests. Clinical tables 615 are also input with database extracts for patient demographics, hospital visits, laboratory measurements, etc. At block 610, patient liver function test values (e.g., represented in patient blood test histories) are extracted from electronic medical records, etc., cleaned, quality checked, and organized in a time series format for one or more patients. Such extraction and processing can also apply to other patient information (e.g., lung function, drug efficacy, etc.).

At block 620, a label definition algorithm is executed to assign an adverse event (AE) grade to the set of blood test/liver function values (e.g., ALT, AST, TBILIRUBIN, ALKPHOS, etc.) and create a binary target label for the AI model. Such label definition can also apply to other patient test values (e.g., lung function, etc.).

At block 630, feature engineering is performed. For example, blood test values and/or other values can be normalized to an upper limit of “normal” to aid in feature generation, comparison, and other analysis. Such a “normal” range can be determined for a particular patient, based on particular blood work, other laboratory test, etc. Feature engineering transforms the normalized values into a discretized symbolic representation, such as a modified Symbolic Aggregate Approximation, etc. In certain examples, motifs can be extracted as n-grams from the series of symbol representations, and counts from patient history can be used as features.

At block 640, an AI model can then be trained and evaluated based on the features, etc., to dynamically predict immune-related adverse event risk from patient data. For example, immune-related adverse event risk can be predicted by the model from fixed-length blood test histories, etc. Output can include model performance metrics 625, a data set of AE risk prediction 635, etc. For example, the risk prediction dataset 635 can include immune-related hepatitis adverse event risk prediction for each history of blood test values. That is, for each patient, a risk of developing a hepatitis AE is predicted until a next ICI treatment appointment for that patient based on their blood test history, etc. The dataset 635 can alternatively include risk prediction for pneumonitis, colitis, hospitalization, etc., based on associated patient data training the model.

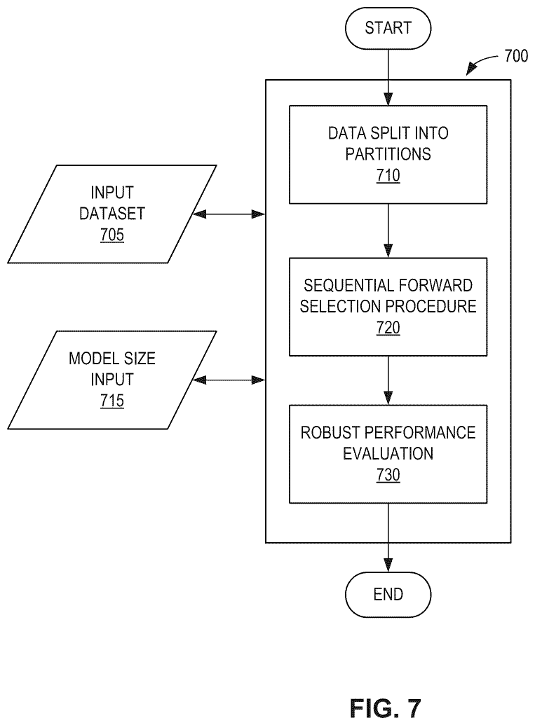

FIG. 7 illustrates an example process 700 for building an example classification model. At block 710, data from an input dataset 705 is divided into a plurality of partitions. For example, structured EHR-derived tabular data, unstructured data tables, aggregated measurement features over time (e.g., 30 days, 60 days, 1 year, etc.), curated labels (e.g., pneumonitis labels, hepatitis labels, colitis labels, etc.), other features (e.g., condition, smoking status, etc.) aggregated over time (e.g., 30 days, 60 days, 1 year, etc.), etc., can be gathered and partitioned. For example, 90% of available data can be organized in a first partition, and the remaining 10% of the data can be organized in a second partition. A model size input 715 can help adjust partitions, etc., based on a typical, expected, or desired number of features in a model.

At block 720, a sequential forward selection procedure is executed to evaluate a plurality of models. The example procedure iterates (e.g., from an initial model to a series of adjusted models) to find patterns in the data to form potential predictive features. For example, the first partition can be evaluated to identify candidate features based on associations between labels and features. A model is selected on each iteration (e.g., an adjusted model), and the evaluation continues.

At block 730, model performance is evaluated. For example, a model selected on a final iteration of the selection procedure 720 is cross-validated, such as on an outer loop of a nested cross-validation using the input dataset 705, to be finalized for deployment and usage.

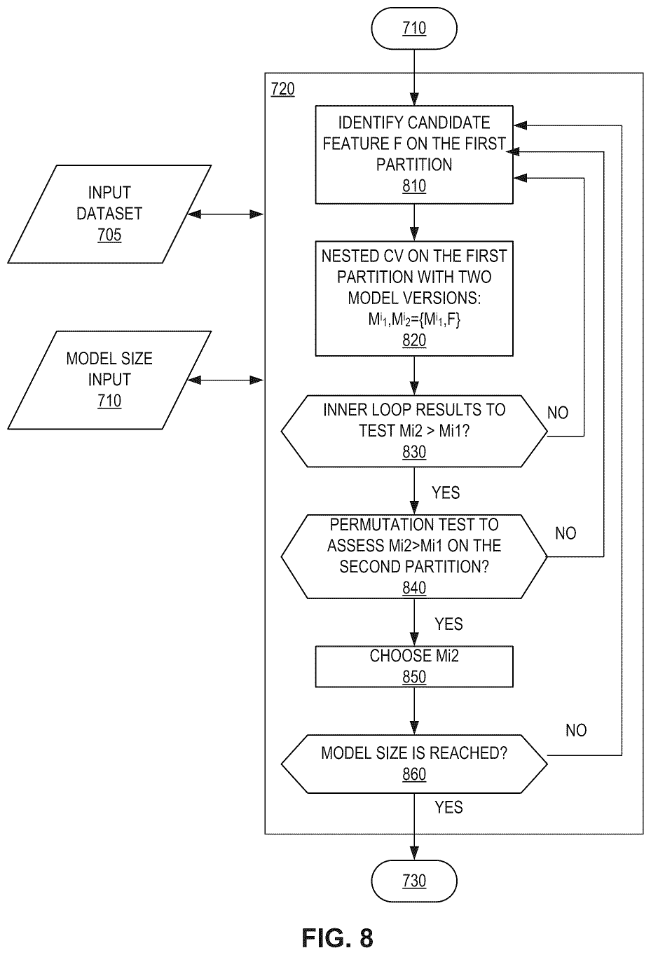

FIG. 8 provides an example implementation of the sequential forward selection procedure 720 of the example process 700 of FIG. 7. At block 810, a candidate feature F is identified in the first data partition. For example, the candidate feature F can be identified based on associations between a target label and existing/identified features. At block 820, a nested cross-validation (CV) is performed on the first partition with two models, M₁ⁱ, M₂ⁱ={M₁ⁱ, F}. That is, model M₂ⁱ includes the features of model M₁ⁱ plus new candidate feature F.

At block 830, inner loop results are evaluated to test whether M₂ⁱ is better than M₁ⁱ. For example, a binomial test is performed to assess whether M₂ⁱ is better than M₁ⁱ based on the inner loop results. If not, then control returns to block 810 to identify another candidate feature in the first partition. When M₂ⁱ is better than M₁ⁱ, at block 840, a permutation test is executed to assess whether M₂ⁱ is better than M₁ⁱ on the second partition. For example, M₂ⁱ and M₁ⁱ can be evaluated based on a p-value, which represents a fraction of permutations of the candidate feature F in which the performance of M₁ⁱ is not smaller than the performance of M₂ⁱ. When M₂ⁱ performs “significantly better” than M₁ⁱ. (e.g., with a standard significance level defined as 0.05 or 0.1), then, at block 850, model M₂ⁱ is selected. Otherwise, control returns to block 810 to identify another candidate feature in the first partition.

At block 860, model size is evaluated (e.g., by comparing current feature set to the model size input 715, etc.). If model size has been reached, then the process can advance to block 730. However, if the model size is not yet met, control returns to block 810 to select a new candidate feature in a next iteration of the example process.

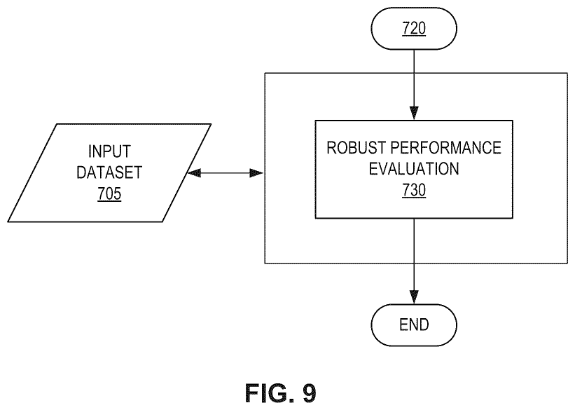

FIG. 9 provides an example implementation of the robust performance evaluation 730 of the example process 700 of FIG. 7. At block 730, performance of the final selected model M₁ⁱ or M₂ⁱ is evaluated using nested cross-validation (CV). As shown in the example of FIG. 9, the input dataset 705 serves as an external test set to validate performance of the selected model and its features. As such, an inner loop of partitioned data can be used to evaluate various candidate models, and an outer loop of test data can be used to validate the performance of the selected model, for example.

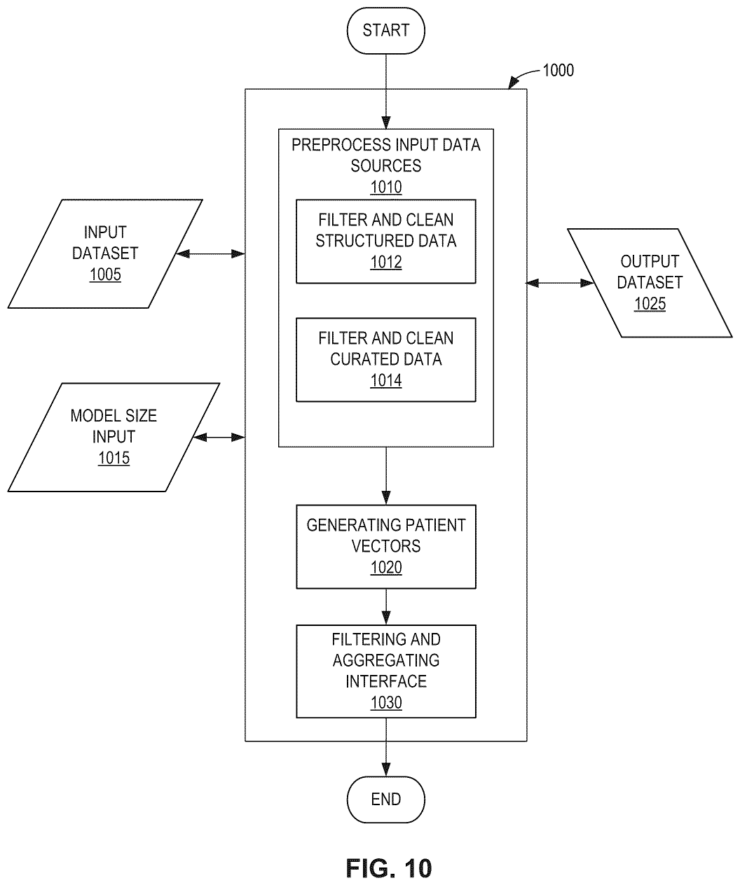

FIG. 10 illustrates an example process 1000 to prepare data from a plurality of sources for use in machine learning and/or other AI model training. For example, database extraction and/or expert curation can be used to generate data from/in an input dataset 1005. Data can be gathered from multiple structured and/or unstructured data sources to form the dataset 1005.

At block 1010, one or more input data sources 1005 are preprocessed to prepare the input data (e.g., by cleaning and extracting features from the data, labeling the features, etc.). Input data and resulting features can include smoking history, drug administration, medical condition(s), radiotherapy history, laboratory measurement(s), anthropometric data, etc. For example, at block 1012, structured data (e.g., drug administration, medical conditions, laboratory measurements, clinical procedures, anthropometric data, etc.) from the input dataset 1005 is filtered and cleaned in a common data format (e.g., a common data model such as an OMOP data model format, etc.). At block 1014, curated data (e.g., related to downstream tasks, smoking history, specific condition history, specific medical history, etc.) from the input dataset 1005 is filtered and cleaned to the common data format.

The preprocessed data set forms an input to block 1020, at which patient vectors are generated from the filtered, cleaned aggregated data set. For example, a model-agnostic set of aggregated patient vectors can be provided with feature filtering. Model-agnostic unaggregated tables having temporal data structure can be used for feature filtering and vector generation. The patient vectors and/or the unaggregated tables can form part of an output dataset 1025.

At block 1030, an interface filters and aggregates data. For example, data can be filtered and arranged around an anchor point and/or other reference, for aggregation and formation into patient vectors.

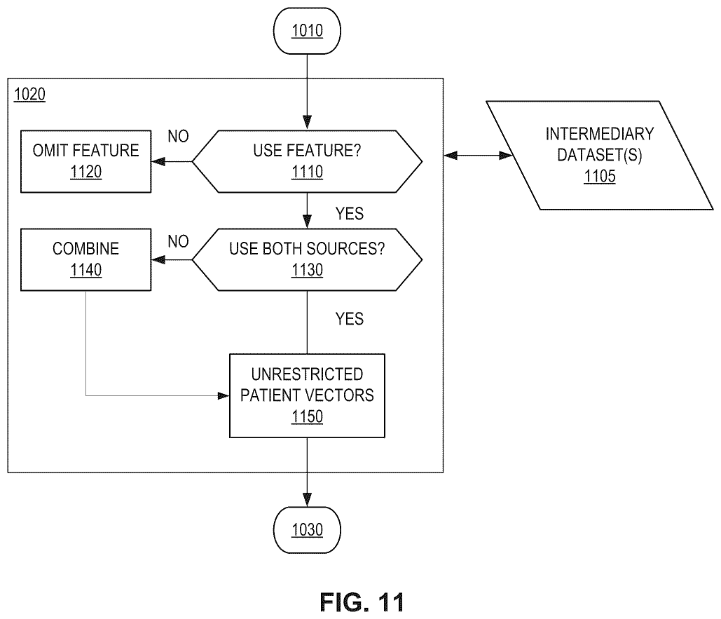

FIG. 11 provides an example implementation of generating patient vectors (e.g., block 1020 of the example process 1000). At block 1110, processed (e.g., filtered, cleaned, etc.) data is evaluated to determine whether a feature in the data is to be used? When the feature is not to be used, at block 1120, the feature is omitted. When the feature is to be used, at block 1130, available data (sources) are evaluated to determine whether multiple sources (e.g., structured and/or curated data sources, etc.) are to be used. When multiple sources are to be used, at block 1140, features from multiple data sources are combined and harmonized. At block 1150, unrestricted patient vectors are formed from downstream model-agnostic unaggregated tables retaining temporal structure. An intermediary dataset output 1105 can include one or more datasets separated by input domain and retaining temporal structure, for example.

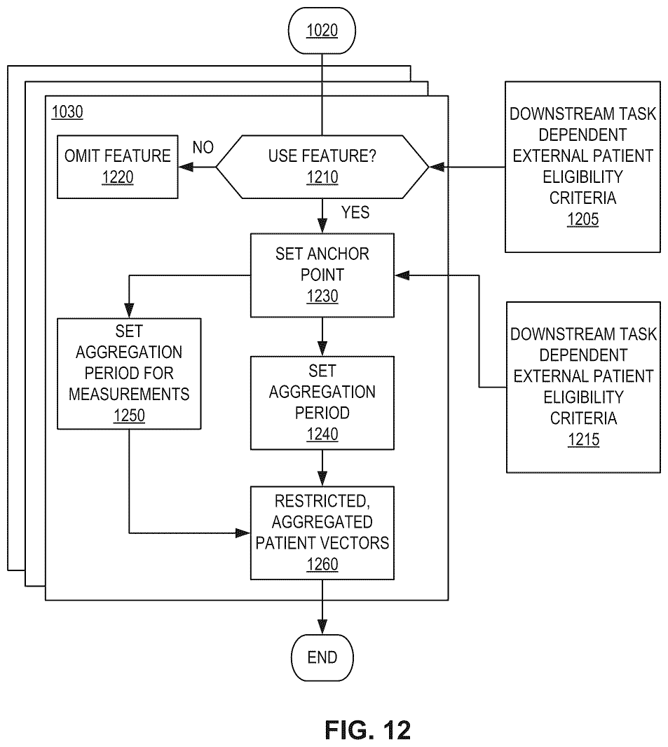

FIG. 12 provides an example implementation of a filtering and aggregating interface (e.g., block 1030 of the example process 1000). At 1210, a patient is evaluated to determine whether the patient is patient for further evaluation and processing. Such a determination can be based at least in part on downstream task-dependent external patient eligibility criteria 1205, for example. For example, does the patient satisfy certain health criteria, age criteria, condition criteria, likelihood of success, etc. When the patient is not eligible, at block 1220, the patient is omitted from further processing.

When the patient is eligible, at block 1230, an anchor point is set for further evaluation. Determination of the anchor point can be based on downstream, task-dependent, external patient eligibility criteria 1215, for example. The anchor point is a patient timeline event that aligns a plurality of patients for comparison. For example, events in a patient's life and/or medical history such as diagnosis, symptom, birth, hospitalization, initial treatment (e.g., date of first immunotherapy treatment), etc., can drive anchor point selection by the framework to align time series data for relational processing and comparison. The anchor point can be a configurable parameter dependent on downstream tasks 1205, for example.

At block 1240, an aggregation period is set. For example, the framework can configure and/or otherwise set an aggregation period before a prediction point (e.g., the anchor point and/or other point in time and/or data for the patient). For example, an aggregation period can include an aggregation of patient data 200 days before a prediction time, etc.

At block 1250, an aggregation period for measurements is set. For examples, the interface framework allows establishment of a different aggregation period for laboratory measurements, other measurements, etc., since such measurements can be more dynamically changing.

At block 1260, one or more restricted, aggregated patient vectors are formed as an output of the interface framework. Patient vectors can be tailored to multiple downstream tasks, for example. In certain examples, for a given set of inputs, patient vectors can be dynamically generated as downstream tasks change.

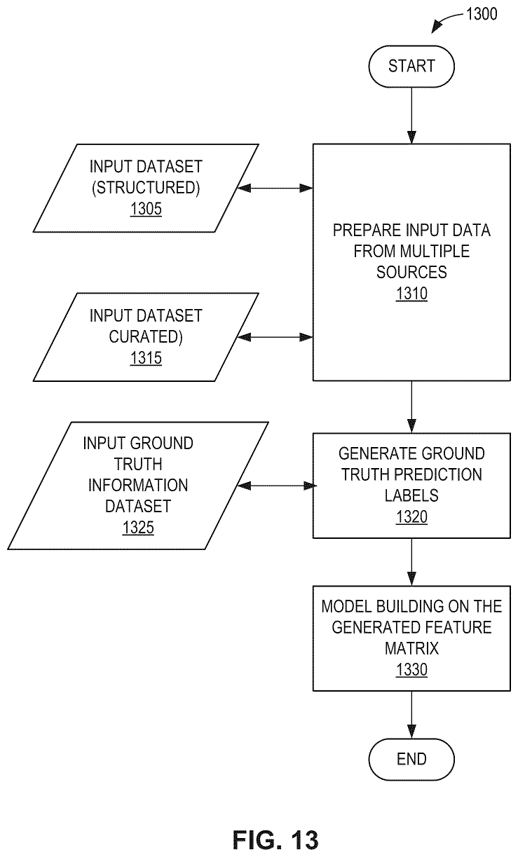

FIG. 13 illustrates an example process 1300 to build predictive models for efficacy related to ICI. Such models can assess multiple efficacy surrogate endpoints to predict a response to ICI therapies. Models can be created for separate cancer indications and/or combinations thereof.

At block 1310, input data from multiple sources is prepared for processing. For example, as described in more detail above, data can be cleaned and features extracted from sources such as a structured dataset 1305, a curated data set 1315, etc. For example, raw, automatically derived EHR data can be converted to a common format with curated data to form a set of features.

At block 1320, ground truth prediction labels are generated. Prediction labels can include time on ICI treatment (TOT), time to next treatment (TNET) (e.g., after discontinuation of ICI), overall survival (OS), etc. The listed ground truth endpoints can be derived from an input data 1325, for example, and can be generated on a continuous scale, such as expressed in days elapsed from an anchor point. For example, patient timelines can be aligned based on similarities in a course of ICI treatment. A default anchor point can be a first date of ICI treatment initiation, for example. Generated ground truth labels can be used as-is and/or with modified granularity (e.g., elapsed weeks, months, years, etc.) for training regression, survival analysis-based models, etc. Discretization of ground truth can be executed for binary and/or multi-class classification (e.g., responders versus non-responders, 5-year survival, etc.).

At block 1330, model building is conducted on a generated feature matrix in which the ground truth serves as a standalone module of the framework. In certain examples, efficacy endpoints can be modeled separately (e.g., individually, etc.). Modeling can be executed hypothesis-free and/or with different machine learning algorithms, for example. Alternatively or additionally, modeling can be done on a continuous scale with survival analysis methodology. Further, modeling can follow a multiclass classification methodology for all endpoints, for example. Example model building and selection is described further above.

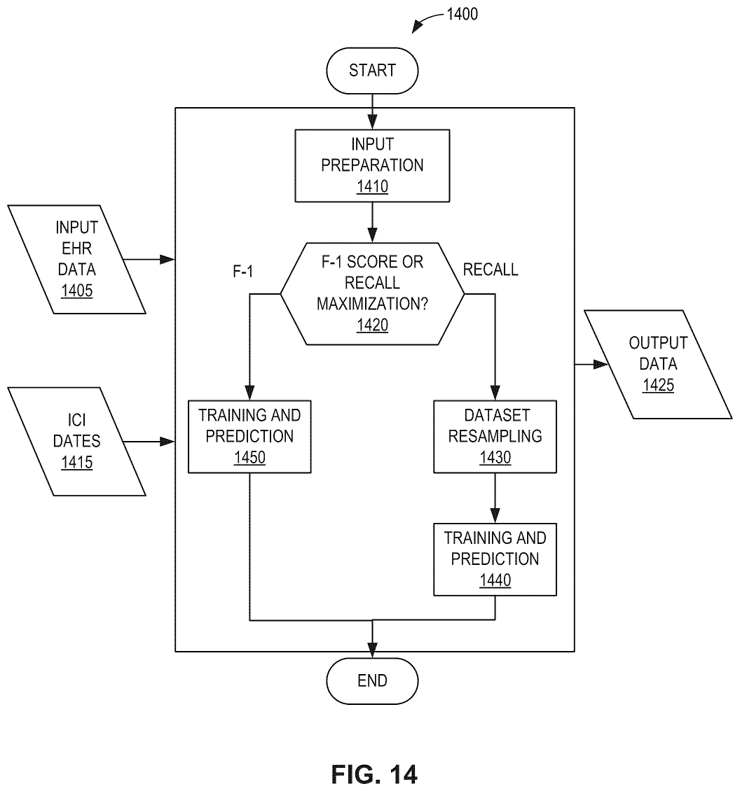

FIG. 14 illustrates an example method 1400 for model selection and generation. In the example of FIG. 14 model generation is driven at least in part by a purpose, goal, or target for the model (e.g., referred to herein as a model selection criterion). For example, the goal may be to generate a model having a high (e.g., maximized) F1 harmonic score (e.g., harmonic mean of recall and precision) such as a GB model, a model having high (e.g., maximized) recall (e.g., almost no false negatives) such as a RF model, a model having high precision (e.g., very few false positives), etc. In certain examples, majority class undersampling can be combined with time series data aggregation to obtain a well-balanced and static dataset to be fed to the models. In certain examples, models create probability estimates for labels, rather than only discrete labels themselves.

At block 1410, input is prepared, such as by extracting data from one or more EHR data tables 1405. Measurements such as liver biomarker concentration in blood plasma, other blood concentration values, etc., can be extracted. Time series measurement and/or other data can be put into a single complex data structure (e.g., aggregation), for example. Aggregated data can be processed using feature engineering to describe the time series data according to mean, standard deviation, minimum, maximum, etc. Lag and features and labels can be created, for example. In certain examples, a date of ICI treatment 1415 is generated (e.g., as a reference or anchor point) to help align and anchor the time series data.

At block 1420, a target or goal for the model(s) is evaluated. As described above, a goal of F1 maximization produces a different trained model (e.g., a gradient boosted model such as gradient-boosted decision tree, etc.) than a goal of recall maximization (e.g., a random forest model, etc.). Based on the determination, for recall maximization, at block 1430, dataset resampling occurs to balance unbalanced dataset from block 1410. For example, random majority class undersampling is performed on the dataset when the goal is to maximize the recall. When the goal is to maximize F-1 score, then resampling can be disregarded.

At block 1440 and/or 1450, a model is trained. For example, an RF model is trained for recall maximization at block 1440, and a GB model is trained for F1-score maximization at block 1450. The respective model is validated such as with leave-one-out cross-validation, for example, in which each sample is predicted individually with the rest serving as a training dataset. The model is trained on a collection of historical data from a plurality of healthcare records, for example.

As such, certain examples generate models that can be deployed, loaded, and utilized to generate predictions to drive patient treatment, adjust patient treatment, configure a cohort for a clinical trial, etc. As described above, nested cross-validation can be used with multiple loops to train and select models. For example, an inner loop can be used for hyperparameter tuning, such as starting from all data points in a patient's history and reducing with feature elimination or using a baseline set of features to which features are added and tested with statistical analysis to determine if the new model performed better than the old one. In the latter case, when an added feature improves performance of the model, that updated model becomes the new baseline, and iteration can continue through available features to arrive at a preferred model for a given set of features. While using all available data points from a patient record can result in tens of thousands of inputs, baseline evaluation can yield 10-12 beneficial feature inputs for effective model training before seeing diminishing returns from inputs being added.

Alternatively or additionally, model types can be evaluated. For example, a multilinear regression model can be generated and compared against a more complex model, such as a random forest model, to determine whether a balance of precision and recall (and/or metric) was better in one model over another. One or models can then be selected for usage in a particular scenario (e.g., for clinical trial or patient treatment, for certain toxicity or for efficacy-related measurement, etc.).

Certain examples generate and deploy particular models to generate particular output predictions from a set of patient healthcare data from one or more patient records. Since the models are trained and tested targeted toward a particular prediction, the models do not work for general prediction. For example, a model cannot successfully predict any toxicity (e.g., including any of pneumonitis, colitis, hepatitis, etc.). Such a model instead generates random chance. However, a model targeted at predicting colitis, or at predicting pneumonitis, or at predicting hepatitis, etc., from an immunotherapy can generate a reliable, actionable prediction to drive cohort configuration for clinical trial, establish and/or change a patient's treatment plan, etc., as described above.

### Example Model Deployment and Usage Systems and Associated Methods

In certain examples, a clinical system can leverage one or more deployed models to drive a tool to evaluate efficacy and/or toxicity associated with immunotherapy for a patient and/or patient population, etc. Using one more deployed models, a patient can be assessed at the start of an immunotherapy treatment plan, during administration of the immunotherapy treatment plan, as a candidate for an immunotherapy clinical trial, etc. In certain examples, prior efficacy and/or toxicity model predictions for the patient can be used with updated efficacy and/or toxicity model predictions for the patient (e.g., in combination between prior and current results, with prior as an input to produce current results, etc.). As such, models are trained and tested using a collection of historical healthcare data across a variety of patients and then applied to a given patient to evaluate outcome(s) and treatment(s) for that patient.

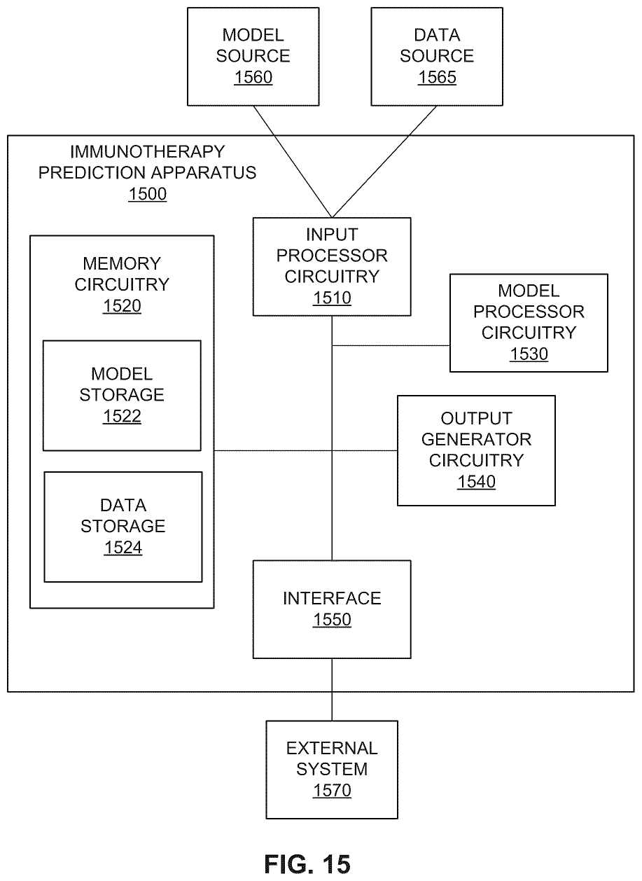

FIG. 15 illustrates an example immunotherapy prediction apparatus 1500 including example input processor circuitry 1510, example memory circuitry 1520, example model processor circuitry 1530, example output generator circuitry 1540, and an example interface 1550. As shown in the example of FIG. 15 the example input processor circuitry 1510 processes input from a model source 1560 (e.g., a model generator apparatus, model repository, EHR, EMR, etc.) as well as a data source 1565 (e.g., an EHR, EMR, laboratory system, clinical information system, scheduling system, etc.).

The input can come at different times, for example, from the model source 1560 and the data source 1565. For example, one or more models can be periodically obtained (e.g., via push and/or pull, etc.) from the model source 1560 and stored in model storage 1522 of the memory circuitry 1520. Patient data and/or other input data can be obtained from the data source 1565 periodically, according to a schedule (e.g., according to a scheduled exam or other appointment, etc.), on demand as requested by a user (e.g., a clinician, administrator, triggered by an examination record, etc.), etc., to be stored in a data storage 1540 of the memory circuitry 1520 to be provided to one or more models by the model processor circuitry 1530, for example. Input data can include structured data (e.g., admission information, discharge information, prescription information, billing codes, diagnosis codes, labs, etc.) and unstructured manually curated medical record data, images, etc.

Model(s) stored in the model store 1522 have been trained and validated by another system, such as the model generation apparatus 100. One or more models can be selected for use in the example apparatus 1500 by the model processor circuitry 1530 and/or by the input processor circuitry 1510 based on input patient and/or other clinical data, for example. Models can be selected according to a variety of model selection criterion. For example, a model can be selected to predict a likelihood of a toxicity, such as pneumonitis, hepatitis, colitis, etc., occurring due to immunotherapy according to a treatment plan for a patient. Alternatively or additionally, a model can be selected to predict an efficacy for immunotherapy treatment for the patient. For example, a model can be selected to initially determine efficacy of an immunotherapy treatment plan for the patient. The same and/or a different model can be selected to determine an ongoing efficacy of the immunotherapy treatment plan for the patient. A model can be selected to evaluate the patient's suitability for an immunotherapy clinical trial, for example.

For example, models can include a toxicity-related prediction model (e.g., a hepatitis prediction model, a colitis prediction model, a pneumonitis prediction model, etc.), an efficacy-related prediction model (e.g., immunotherapy efficacy model, survival model, time on treatment model, etc.), etc. The models can be high recall with low precision, low recall with high precision, harmonic mean (F1) maximized, majority undersampling, etc. A selection can be made by and/or for the example model processor circuitry 1530 (e.g., based on a setting, mode, type, query, patient identifier, other model selection criterion, etc.). For example, when configuring participation in a drug trial, a focus is on F1 score maximization to identify patients with a highest chance to respond well to a given immunotherapy drug. When determining a clinical treatment for a patient, however, a goal is to eliminate as many toxicities as possible for the patient. As such, a model developed with high recall is more important, and low precision is acceptable because of inexpensive treatment options for hepatitis, colitis, pneumonitis, etc. The goal may also depend on patient preference (e.g., desire for aggressive treatment, desire for quality of life, adjustment depending on life event, etc.), for example. Such a model can also be evaluated according to a particular threshold or other patient selection criterion,

The model processor circuitry 1530 processes input data (e.g., from the input processor circuitry 1510, the data storage 1522, etc.) using one or more selected models (e.g., from the input processor circuitry 1510, the model storage 1522, etc.). The model processor circuitry 1530 produces one or more predictive outputs based on one or more provided inputs. The output and/or other content can be processed by the output generator circuitry 1540 for output to an external device or system 1570 via the interface 1550. For example, the output generator circuitry 1540 can combine prediction(s) of toxicity and/or efficacy, together and/or further with images, explanation, treatment plan information, patient data, clinical trial information, etc.

In operation, the example input processor circuitry 1510 processes input patient data related to one or more patients such as laboratory results, diagnosis codes, billing codes, etc. Input can originate at one or more external systems 1565 such as an EHR, EMR, etc. The example input processor circuitry 1510 can extract and organize the input in a time series for a patient, for example. In certain examples, the input processor circuitry 1510 aligns the input data with respect to an anchor point (such as date of first disease symptom, date of diagnosis, date of first immunotherapy treatment, etc.) to organize the input data in the time series.

In certain examples, the time series data is formed by the input data processor 1510 into a plurality of features. These features can form a set of patient features identifying and/or categorizing healthcare data that are input to one or more models using the model processor circuitry 1530. Feature engineering by the input data processor 1510 can form a plurality of features based on codes (e.g., ICD-10 codes, etc.), for example. For example, ICD-10 codes for a patient in a given year can be processed to identify codes relevant to lung and/or respiratory function (C34, C78, etc.), and the time series of those codes can form a function used to calculate a relative likelihood of lung disease in the patient. Similarly, a plurality of features can be formed to predict development of pneumonitis, hepatitis, colitis, and/or other toxicity from immunotherapy treatment. Features can be formed based on lung function, liver biomarkers, blood work (e.g., concentration in blood, plasma, etc.), drugs taken, etc.

Patient features and/or other patient input are applied to one or more selected models by the model processor circuitry 1530. In certain examples, a single feature input set or string is provided to a model (e.g., a feature set of ICD-10 codes for the patient, etc.). In other examples, multiple inputs including multiple features, a prior model output prediction (e.g., a prior prediction of efficacy and/or toxicity input to the model for an updated prediction), a different model output prediction (e.g., providing an efficacy model prediction output as input to a toxicity model, providing a toxicity model prediction output as input to an efficacy model, etc.), etc., are applied to one more models. The model processor circuitry 1530 generates an output from the model(s) based on the input.

Output from the model(s) of the model processor circuitry 1530 is provided to the output generator circuitry 1540, which processes the prediction and/or other output of the model(s). For example, output from multiple models of the model processor circuitry 1530 can be compared by the output generator circuitry 1540 to form a resulting output to provide to the interface 1550, another system, etc. The output generator circuitry 1540 can post-process the output to validate the output, compare a current output against prior and/or other current output predictions, provide feedback to the model source 1560, reformat the output, etc. In certain examples, the output generator circuitry 1540 can correlate the model output with other data, such as image data, etc., to produce a qualified or refined output and/or other correlated/verified result.

In certain examples, an output of the model(s) is explainable, such as by providing an indication of the input feature(s), rule(s), model layer, etc., that resulted in the output prediction. The output generator circuitry 1540 can leverage the explanation accompanying the output to drive decision making and actionable output related to a treatment plan, clinical trial, and/or other next step for the patient, for example.

In certain examples, a patient selection criterion, such as a threshold, can be applied to the model output. For example, the model output can represent a probability of a certain outcome (e.g., a probability or likelihood of a certain experiencing a certain toxicity during the immunotherapy treatment, a probability of treatment success or efficacy, etc.) based on the data and associated features and correlations used to generate the model. A patient selection threshold can be used to set a point in the continuum of the model output at which a trigger or determination of action or event occurs. That is, if the predicted model output value falls on one side of the threshold, then one action happens (e.g., the patient is included in the immunotherapy, etc.), but, if the model predictive output falls on the other side of the threshold, then another action happens (e.g., the patient is excluded from the treatment). As such, a model may provide a binary prediction (e.g., in or out), for example. Alternatively, a model may provide a multiple outcome prediction (e.g., no, yes, yes with caveats, etc.), for example.

The output generator circuitry 1540 can incorporate the output prediction of efficacy and/or toxicity into an immunotherapy treatment plan for the patient, for example. Alternatively or additionally, the output generator circuitry 1540 can serve as a trigger to include or exclude the patient from a clinical trial or study based on the output, for example. The clinical trial can be thought of as a treatment plan involving the immunotherapy for a cohort of multiple patients, for example. The output generator circuitry 1540 can utilize the output as a trigger to modify an existing immunotherapy treatment plan (e.g., to continue, stop, increase, decrease, etc., administration of immunotherapy drug(s) to the patient, etc.) for the patient such as based on an increased probability of toxicity, decreased probability toxicity, increased efficacy, decreased efficacy, etc. In certain examples, the prediction drives a modification of the treatment plan to address an increased likelihood of toxicity, such as prescribing steroids to treat pneumonitis in a patient while continuing the course of immunotherapy treatment, pre-treating for a predicted onset of hepatitis based on a determined likelihood of liver toxicity, etc. In certain examples, the output generator circuitry 1540 generates an alert and/or otherwise provides decision support to effect change to a treatment plan, clinical trial, etc. Current and prior predictions along with old and new data points can drive treatment plans, adjustments, updated models, etc., in a dynamic, looping system.

The output generator circuitry 1540 can store output in the data storage 1524, for example. The output generator circuitry 1540 provides an output for transmission via the interface 1550, such as graphically to the external system 1570, as an input/command/setting for configuration of the external system 1570 (e.g., to activate a treatment plan, adjust a treatment plan, form a cohort of patients for a clinical trial, initiate a clinical trial, etc.), and/or other actionable output.

In certain examples, the output generator circuitry 1540 triggers an indication (e.g., a visual indication on a display, a textual indication in a patient record or report, an audible alert) corresponding to processing of the predictive output for the patient. The indication can differ based on whether the patient is recommended/accepted/selected (e.g., included in a clinical trial, selected for new/ongoing treatment, etc.) versus declined/removed (e.g., exclude from a clinical trial, declined for new treatment, removed from current treatment, etc.). The output can also include an associated action such as to include the patient in the cohort for the clinical trial, exclude the patient from the clinical trial, generate an order for the patient to begin immunotherapy treatment, cancel an order for ongoing immunotherapy treatment, connect a clinician and/or patient to a scheduling and/or ordering system, update patient record, etc.

As such, the example apparatus 1500 is a digital tool that can be used to select patients for clinical trial as well as to develop and deploy therapeutics and monitor treatment of a patient. The example apparatus 1500 enables toxicity potentially associated with immunotherapy, such as hepatitis, pneumonitis, colitis, etc., to be evaluated by one or more models while also evaluating efficacy of the immunotherapy with respect to a particular patient (e.g., likelihood of patient survival (with and/or without immunotherapy), progression-free survival, time on treatment, etc.).

The example apparatus 1500 can provide a plurality of predictions over time for the patient (e.g., periodically, at certain milestones, as the patient's condition and/or response to the treatment evolves, etc.). Comparison of multiple predictions by the apparatus 1500 enables assessment of a risk versus benefit ratio for the patient with respect to the immunotherapy treatment plan. Based on the ratio, treatment can be continued or increased when the benefit outweighs the risk and reduced or ceased when the risk outweighs the benefit, for example. In certain examples, a plurality of model prediction outputs can be compared to determine trends, update models, drive initiation and/or change to a treatment plan, clinical trial, etc.

For example, an evaluation to configure a cohort for a clinical trial may only use a toxicity-related model because efficacy may not yet be known (or sufficient efficacy-related data may not be available to make a reliable prediction) and/or because a pharmaceutical company may be focused on reducing toxicity regardless of predicted efficacy. An evaluation regarding a course of treatment for a patient may use a combination of a toxicity-related model and an efficacy-related model, as weighted or modified by one or more patient selection criterion or factors (e.g., patient criticality, disease severity, patient willingness, lifestyle, overall patient health or co-morbidity, etc.), to determine whether a balance of toxicity risk and treatment efficacy weigh in favor of or against starting the patient on a treatment regime, continuing the treatment regime, joining a clinical trial cohort, etc.

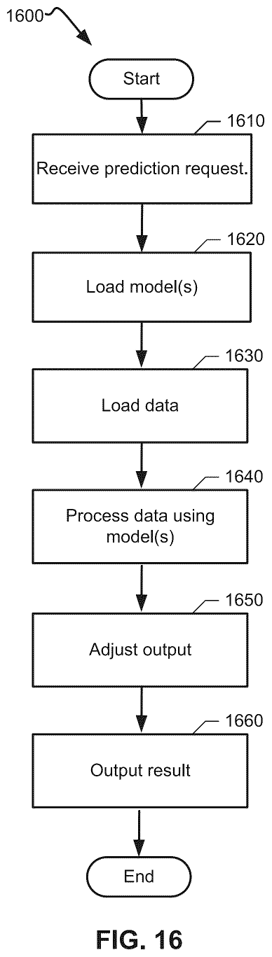

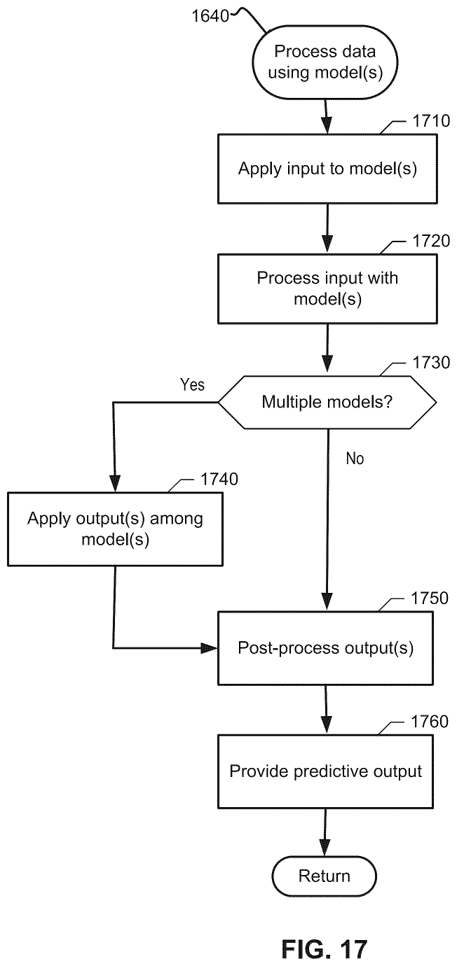

FIGS. 16 and 17 are flow charts of example processes representing computer-readable instructions storable in memory circuitry and executable by processor circuitry to implement and actuate the example immunotherapy prediction apparatus 1500 of FIG. 15. The example process 1600 of FIG. 16 begins at block 1610, at which a request for prediction and/or other processing trigger is received by the example prediction apparatus 1500. For example, a request for prediction modeling output is received via the interface 1550 (e.g., by user selection via a graphical user interface, by initiation of a software program, via the input processor circuitry 1510, otherwise from an external system 1570, etc.). The request can include a toxicity prediction associated with a plan for immunotherapy treatment (“an immunotherapy treatment plan”), an efficacy prediction for the immunotherapy treatment plan, a likelihood of successful inclusion in an immunotherapy clinical trial, etc.

At block 1620, one or more models are loaded from the model storage 1522 and/or external model source 1560, etc., for processing according to the request by the model processor circuitry 1530. For example, a RF, GB, and/or other model can be loaded based on the prediction desired and/or otherwise triggered by the request (e.g., toxicity, efficacy, eligibility, initial vs. in progress, etc.). A model selection criterion, such as model type, objective (e.g., a target or goal such as inclusive trial, exclusive trial, easy treatment, aggressive treatment, likely success, speculative success, low risk, high risk, etc.), etc., can be used to select model(s) for loading and use, for example. At block 1630, patient and/or other healthcare data to be input into the model is loaded for the model processor circuitry 1530 (e.g., from the data storage 1524, external data source 1565, other patient healthcare record, etc.).

At block 1640, the healthcare data for the given patient is processed using the selected model(s). For example, the model processor circuitry 1530 inputs the data to the selected model(s), which generate output. For example, based on codes and/or other patient data associated with lung function, liver function, blood work, etc., the model(s) determine a likelihood of hepatitis, pneumonitis, colitis, and/or other toxicity for the patient on the course of immunotherapy treatment. Alternatively or additionally, the model(s) can process the input to determine a likelihood of immunotherapy efficacy to drive prescription of a treatment plan, adjustment of a treatment plan, selection for clinical trial, etc.

At block 1650, the output can be adjusted, such as via post-processing, comparison, additional model output, etc. For example, output of one or more models from the model processor circuitry 1530 is further processed by the model processor circuitry 1530 to apply another model, compare model output, scale/refine/otherwise select model output, etc., and/or by the output generator circuitry to form an actionable output from the model prediction(s).

In certain examples, the output is compared to a threshold or other patient selection criterion to determine an associated next action. For example, a patient selection criterion can be determined based on a desired relationship or balance between precision and recall for the model (e.g., a high threshold to include, an intermediate threshold to include, a low threshold to include, etc.). As such, the model output can be evaluated with respect to the patient selection criterion and different indications and associated actions can be generated (e.g., as an actionable result or output) based on the comparison. For example, if the model predictive output satisfies the patient selection criterion (e.g., exceeds the threshold), then a first indication and associated action to include the patient (e.g., in the clinical trial, in the treatment, to continue the treatment, etc.) are generated. However, when the model predictive output does not satisfy the patient selection criterion (e.g., does not meet the threshold), then a second indication and associated action to exclude the patient (e.g., from the clinical trial, from the treatment, to discontinue treatment, etc.) are generated.

The patient selection criterion can be provided in the form of a value that divides the output into greater than, less than, greater than or equal to, less than or equal to, etc. The patient selection criterion can establish a lower boundary value or threshold, an upper boundary value or threshold, a range of acceptable or unacceptable values (e.g., with a maximum threshold and a minimum threshold), etc. The patient selection criterion can be determined based on an analysis of model characteristics, such as precision and recall graphs associated with the output of the model, for example. As such, characteristics of the particular model, taken alone or in combination with external/additional concerns for patient care, clinical trial development, etc., can be processed to generate a patient selection criterion (e.g., the in form of one or more single thresholds, associated ranges, etc.) to trigger a next action based on where the model prediction for a given patient falls with respect to the patient selection criterion.

At block 1660, an actionable result is provided. For example, the output generator circuitry 1540 generates a visual output for the interface 1550 from the processed prediction of the model(s) from the model processor circuitry 1530. As another example, an instruction and/or prescription for a new immunotherapy treatment plan and/or for modification of existing immunotherapy treatment plan can be output to the external system 1570 by the output generator circuitry 1540 via the interface 1550. As another example, an instruction and/or notification/alert/indication to include the patient in an immunotherapy clinical trial or remove the patient from the immunotherapy clinical trial can be output to the external system 1570 by the output generator circuitry 1540 via the interface 1550. Display of an indication and instruction to include/exclude, etc., can form an order generated by the output generator circuitry 1540 and provided via the interface 1550 to drive action at another system (e.g., scheduling, management, clinical care system, clinical trial database, etc.) with respect to the patient/patient record, for example. The output or indication can be driven by a comparison of the prediction to a threshold or other patient selection criterion, for example.

FIG. 17 illustrates further detail for an example implementation of processing input data using one or more models (e.g., block 1640 of the example process 1600). At block 1710, pre-processed input is applied to one or more selected model to, at block 1720, process the input. For example, input patient data related to one or more patients such as laboratory results, diagnosis codes, billing codes, etc., can be aligned in a time series with respect to an anchor point (e.g., a reference event or point in time in the patient's health history and record) and applied to the model(s) of the model processor circuitry 1530.

For example, the model processor circuitry 1530 inputs the data to the selected model(s), which generate output. For example, based on codes and/or other patient data associated with lung function, liver function, blood work, etc., the model(s) determine a likelihood of hepatitis, pneumonitis, colitis, and/or other toxicity for the patient on the course of immunotherapy treatment. Alternatively or additionally, the model(s) can process the input to determine a likelihood of immunotherapy efficacy to drive prescription of a treatment plan, adjustment of a treatment plan, selection for clinical trial, etc.

In certain examples, the time series data is formed by the input data processor 1510 into a plurality of features. These features can form a set of patient features that are input to one or more models using the model processor circuitry 1530. Feature engineering by the input data processor 1510 can form a plurality of features based on codes (e.g., ICD-10 codes, etc.), for example. For example, ICD-10 codes for a patient in a given year can be processed to identify codes relevant to lung and/or respiratory function (C34, C78, etc.), and the time series of those codes can form a function used to calculate a relative likelihood of lung disease in the patient. Similarly, a plurality of features can be formed to predict development of pneumonitis, hepatitis, colitis, and/or other toxicity from immunotherapy treatment. Features can be formed based on lung function, liver biomarkers, blood work (e.g., concentration in blood, plasma, etc.), etc.

Patient features and/or other patient input are applied to one or more selected models by the model processor circuitry 1530. In certain examples, a single feature input set or string is provided to a model (e.g., a feature set of ICD-10 codes for the patient, etc.). In other examples, multiple inputs including multiple features, a prior model output prediction (e.g., a prior prediction of efficacy and/or toxicity input to the model for an updated prediction), a different model output prediction (e.g., providing an efficacy model prediction output as input to a toxicity model, providing a toxicity model prediction output as input to an efficacy model, etc.), etc., are applied to one more models.

At block 1730, selected model(s) are evaluated to determine whether the model(s) include a plurality of related models. For example, the model processor circuitry 1530 evaluates the selected model(s) to determine whether the model(s) include related models for immunotherapy efficacy and toxicity. The model processor circuitry 1530 may also evaluate the selected model(s) to determine whether the model(s) include a current model and a prior model or model output. At block 1740, if there are related models selected for processing, the model processor circuitry 1530 applies one or more outputs between the related models. For example, the model processor circuitry 1530 may apply a prior prediction in comparison with a new model prediction output and/or as an input to a new model. The model processor circuitry 1530 may compare and/or otherwise process efficacy and toxicity predictions, for example.

At block 1750, output from the model processor circuitry 1530 is post-processed. For example, the model processor circuitry 1530 and/or the output generator circuitry 1540 process output(s) of the model prediction(s) to formulate and/or otherwise adjust a treatment plan, formulate instruction associated with treatment plan for clinical care or for clinical trial, etc. Predictive model output(s) can be correlated with image and/or other data, for example. Predictive model output(s) can be compared to a threshold and/or otherwise evaluated with respect to other patient selection criterion (-ia) to translate the prediction into an action to be taken (e.g., automatically and/or by a clinician, etc.). Explanation and/or other actionable information/instructions can be associated with predictive output(s) to make the output(s) actionable by another system, program, device, etc.

At block 1760, the processed, actionable predictive output is provided. For example, the output generator circuitry 1540 can incorporate the output prediction of efficacy and/or toxicity into an immunotherapy treatment plan for the patient, for example. Alternatively or additionally, the output generator circuitry 1540 can serve as a trigger to include or exclude the patient from a clinical trial or study based on the output, for example. The output generator circuitry 1540 can utilize the output as a trigger to modify an existing immunotherapy treatment plan (e.g., to continue, stop, increase, decrease, etc., administration of immunotherapy drug(s) to the patient, etc.) for the patient such as based on an increased probability of toxicity, decreased probability toxicity, increased efficacy, decreased efficacy, etc. In certain examples, the prediction drives a modification of the treatment plan to address an increased likelihood of toxicity, such as prescribing steroids to treat pneumonitis in a patient while continuing the course of immunotherapy treatment, pre-treating for a predicted onset of hepatitis based on a determined likelihood of liver toxicity, etc. In certain examples, the output generator circuitry 1540 provides decision-making, decision support, and/or other notification/alert to affect change to a treatment plan, clinical trial, etc. Current and prior predictions along with old and new data points can drive treatment plans, adjustments, updated models, etc., in a dynamic, looping system. The output generator circuitry 1540 provides actionable output to the interface 1550 for display and/or other distribution to the external system 1570 and/or other connected device, for example.

As such, one or more models can be generated, deployed, and configured for use to predict toxicity resulting from an immunotherapy treatment, efficacy of the immunotherapy treatment, a combination thereof, etc. In certain examples, a model (e.g., a toxicity-related model, an efficacy-related model, etc.) is deployed as part of a program or platform that loads the model and provides healthcare data from a healthcare record for a given patient to that model. An output of the model, as evaluated with respect to a single threshold, range, or other patient selection criterion, is used to configure a treatment plan for the given patient. That treatment plan includes an immunotherapy treatment and may include a cohort of multiple patients forming a clinical trial, for example.

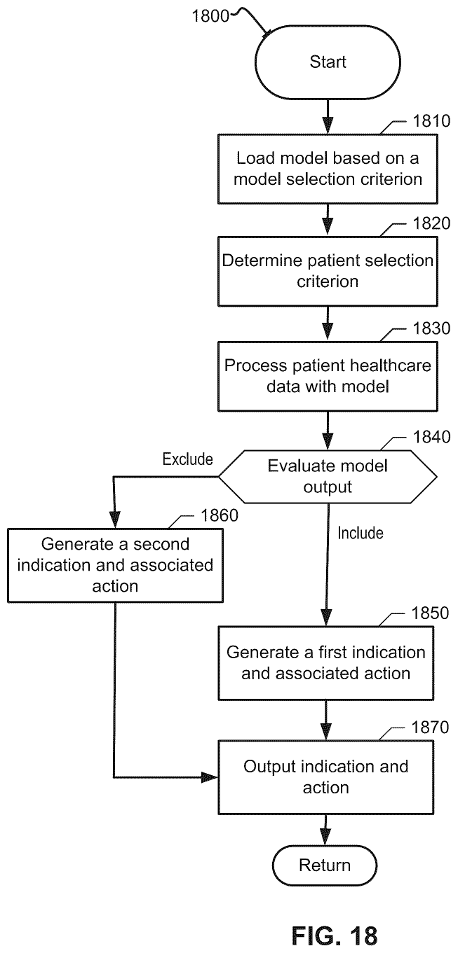

FIG. 18 illustrates another example process 1800 for configuration of a clinical trial or treatment plan associated with a patient. At block 1810, a model is loaded based on a model selection criterion. For example, a model, such as a toxicity-related model (e.g., a pneumonitis toxicity model, a colitis toxicity model, a hepatitis toxicity model, etc.), an efficacy-related model (e.g., a survival model, a time-on-treatment model, etc.), etc., can be loaded based on a model selection criterion such as a permissive or inclusive model target (e.g., err on the side of including a patient), an exclusive model target (e.g., err on the side of excluding the patient), etc. As such, a model can be selected that was trained to emphasize recall over precision, precision over recall, balance precision and recall, etc., depending on the objective or goal, such as including as many patients as possible in a study, restricting the number of patients in the study, aggressively treating a patient with immunotherapy, conservatively treating the patient with immunotherapy, etc.

In certain examples, the objective or motivation can change over time (e.g., treatment starts cautiously for a patient and becomes more aggressive, the patient's circumstances change to warrant a treatment with less toxicity and/or greater efficacy, a drug company does an initial screen and then wants to switch to a broader confirmation, etc.). As such, model loading can be repeated with different model selection criterion over time.

At block 1820, a patient selection criterion is determined. For example, the patient selection criterion can be based on or correlate with the model selection criterion (e.g., both restrictive, both permissive, etc.). However, the patient selection criterion can also diverge or otherwise be different from the model selection criterion. For example, while a model to predict toxicity in a patient from an immunotherapy treatment may be permissive for a clinical trial, the patient selection criteria may adjust to seek a different balance of true positive, true negative, false positive, and false negative to select a patient.

The threshold or other patient selection criterion is used to define a balance between precision and recall associated with a confidence level that the prediction provided by the model is correct. The threshold can be set based on one or more factors, such as a confidence in predicting that one person will not get the toxicity versus a confidence in predicting that another person will get the toxicity. One treatment plan may prioritize being correct that someone will get the toxicity, and another treatment plan may prioritize being correct that someone will not experience the toxicity, for example. Similarly, one treatment plan may prioritize correctly predicting that someone will survive, and another treatment plan may prioritize correctly predicting that someone will not survive. Certain treatment plans, whether for individual patient treatment or for clinical trial, may prioritize a certain minimum precision, a certain minimum recall, or a certain balance between precision and recall to move forward with an action triggered by the prediction, for example.

In certain examples, the patient selection criterion is a threshold set at a number between 0.0 and 1.0. The threshold can be used to determine a next course of action with respect to a patient based on the predictive output of the model. For example, the patient selection criterion can be set at a threshold of 0.5. Then, a first patient with a toxicity prediction of less than 0.5 can be flagged for inclusion in a clinical trial, but a second patient with a toxicity prediction of greater than 0.5 can be flagged for exclusion in the clinical trial. As another example, a threshold can be set for patient selection criterion at 0.3 such that a first patient with an efficacy-related prediction of 0.3 or more is flagged for treatment with an immunotherapy drug while a second patient with an efficacy-related prediction of less than 0.3 is not included.

In certain examples, the objective or motivation can change over time (e.g., treatment starts cautiously for a patient and becomes more aggressive, the patient's circumstances change to warrant a treatment with less toxicity and/or greater efficacy, a drug company does an initial screen and then wants to switch to a broader confirmation, etc.). As such, the patient's healthcare data can be re-evaluated with the same and/or a different model with the output transformed according to a different patient selection criterion and/or a different model selection criterion over time. For example, a patient may not be very sick and just looking for an immunotherapy treatment with low risk of toxicity. If the patient's condition worsens, the goal can switch to looking for a treatment with a high likelihood of efficacy for that patient, regardless of toxicity. A different model can be selected, and updated patient data processed with the updated model, for example.

At block 1830, patient healthcare data is processed using the selected model. For example, healthcare data from a patient's healthcare record (e.g., EMR, EHR, other clinical or healthcare database, etc.) is provided to the selected model (e.g., a toxicity-related model, an efficacy-related model, etc.). The patient data (e.g., bloodwork, prescription history, other biometric and/or lab data, etc.) is processed by the model, which correlates and weights features associated with the patient data to generate a prediction (e.g., predict a likelihood of toxicity from an immunotherapy treatment, predict a likely efficacy of an immunotherapy treatment, etc.). As noted above, the model is trained, tested, and deployed to target a particular prediction, such as a likelihood of a particular toxicity, efficacy of a particular immunotherapy drug, etc., rather than provide a general indication of any toxicity likely to result from having a patient take the drug or a general efficacy of any drug to treat a particular type of cancer, etc., for example. As such, the model provides a particular predictive output using data from the particular patient's medical records.

At block 1840, the model output is evaluated. For example, the model output can be a prediction on a scale from 0.0 to 1.0. The model predictive output can be evaluated with respect to the patient selection criterion, for example. As described above, a patient selection criterion or threshold can be set to allow low precision/high recall (a low threshold such as 0.0-0.3), a balance between precision and recall (an intermediate threshold such as 0.4-0.6), high precision/low recall (a high threshold such as 0.7-1.0), etc. Such a threshold or other patient selection criterion can be selected based on a tolerance or priority for correctly predicting a positive outcome, incorrectly predicting a positive outcome, correctly predicting a negative outcome, incorrectly predicting a negative outcome, etc.

Based on the evaluation of the model output according to the patient selection criterion, a next action is triggered. When the patient's model output satisfies the patient selection criterion, then, at block 1850, a first indication is generated to include the patient in a clinical trial, treatment plan, etc., and an associated action is triggered (e.g., to generate an order to prescribe for the patient, include the patient in a trial or study cohort, modify the patient's treatment plan, etc.). When the patient's model output does not satisfy the patient selection criterion, then, at block 1860, a second indication is generated to exclude the patient in a clinical trial, treatment plan, etc., and an associated action is triggered (e.g., to note in the patient record that the patient is excluded from the trial or treatment, modify the patient's treatment plan, etc.). At block 1870, the indication and associated action are output. For example, the output can be displayed on a screen, messaged to a responsible clinician, messaged to the patient, generated in an order for an immunotherapy drug or other regimen, routed to a scheduling system for an appointment, used to update the patient's medical record or clinical trial cohort, etc.

In certain examples, a decision may not be a treat/don't treat binary decision. Instead, the decision may be to treat the patient according to the immunotherapy for a certain period of time and/or with additional testing, monitoring, etc., to help ensure that the patient does not begin to experience adverse effects from the treatment (and/or is responding well to the treatment). As such, a prior decision/output can be fed back into the model to re-evaluate periodically based on updated patient information, etc.

As described above, a balance between precision and recall can be determined for a single model and/or for a set of related models. In order to make a model output (e.g., a prediction) actionable, the patient selection criterion (e.g., a threshold or other selection criterion) is set, above which a first action is taken and below which a second action is taken. A low threshold or selection criterion provides low precision in the model (e.g., many false positives) but high recall in the model (e.g., almost no false negatives). An intermediate threshold or other selection criterion provides a good balance between false positives and false negatives. A choice of intermediate threshold depends on the characteristics and performance of the particular model so that it can vary from model to model rather than being uniform. A high threshold or other selection criterion provides high precision in the model (e.g., very few false positives) but low recall (e.g., many false negatives).

For example, a predictive output of a single toxicity-related model can be evaluated based on a patient selection criterion/threshold set at a low threshold to accept more false positives (e.g., more predictions of toxicity than actually occur) in the interest of getting more people into a clinical trial. As another example, an efficacy-related model such as an overall survival model can be configured or set according to a patient selection criterion set at a high threshold to allow few false positives.

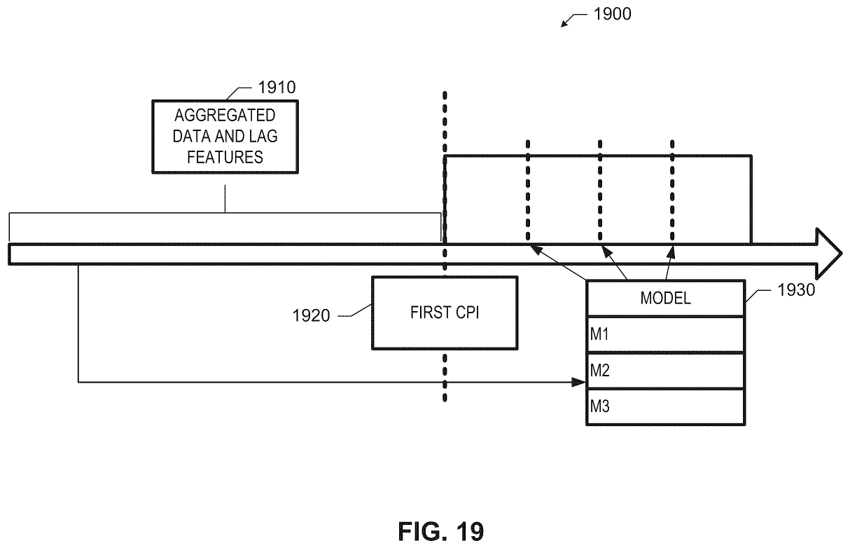

Thus, as set forth above and depicted, for example, in the representation 1900 of FIG. 19, a large volume of aggregated patient-related healthcare data can be processed according to a subset of features associated with the data 1910. When the first immunotherapy checkpoint inhibitor (CPI) is administered 1920, that date 1920 can be used as an anchor point to align the aggregated patient-related data set for use in model training and testing to form a plurality of models 1930 M1, M2, M3, etc. One of the models M1, M2, Me can be selected based on a model selection criterion, such as an emphasis on precision (e.g., how many retrieved items are relevant), an emphasis on recall (e.g., how many relevant items are retrieved), a balance between precision and recall, etc., and deployed for use in an application, platform, service, system, etc.

### Example Model Characteristics and Associated Output

As described above, a model is built to answer a particular question such as what is a patient's risk of a particular toxicity, what is the chance that this immunotherapy is effective for this patient. Such questions, also referred to as targets or objectives, allow available data to be organized and processed to form connections and correlations to predict potential (e.g., likely) answers to those questions. A model takes the data, including results observed in the patient data pool (e.g., this person survived with treatment, this person suffered from colitis while on this treatment, etc.) and forms a network of connections, weights, and other correlations to generate a probabilistic output. As such, the model can turn patient information into probabilities based on a collection of other data and results. Such probabilities can be expressed in terms of precision and recall, for example.

In certain examples, each model can be characterized by a relationship or combination of precision and recall related to its output. Based on characteristics of the model, as illustrated by the precision and recall of the associated model output, a threshold or other patient selection criterion can be set to process or interpret the output of the model. For example, model characteristics or behavior, as reflected in precision and recall curves associated with the particular model, can be evaluated to set the patient selection criterion. If a goal or objective is to reduce or minimize false positives, then the threshold can be set accordingly based on an evaluation of a precision curve associated with the model. If the goal or objective is to reduce or minimize false negatives, then the threshold can be set accordingly based on an evaluation of a recall curve associated with the model. In many instances, a balance of false positives and false negatives leads to an analysis of both precision and recall curves to set the patient selection criterion threshold for use of a model.

Setting the threshold as a patient selection criterion indicates that a certain balance of (or dichotomy between) precision and recall is satisfactory to take action based on the particular model. For example, a clinician or researcher may be satisfied with a certain percentage of errors or inaccuracies in the prediction. Alternatively or additionally, a clinician or researcher may require a certain reliability in the prediction. Such risk tolerance can depend on the patient, the immunotherapy, health concerns, liability concerns, etc.

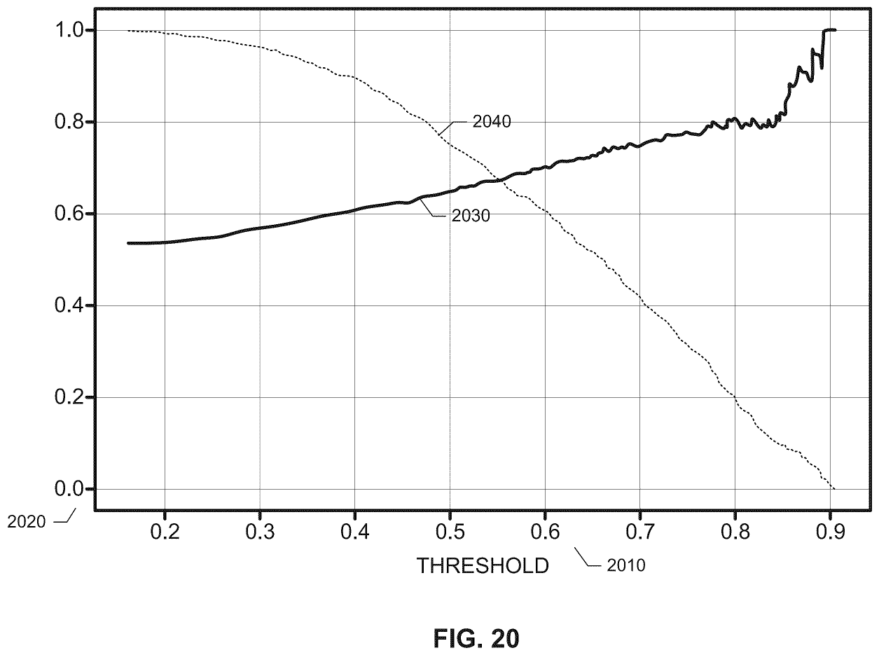

FIGS. 20-24 illustrate example precision/recall graphs related to the reliability of model output. As shown in the following examples, model characteristics are reflected in the graphs, and different thresholds can be set as patient selection criterion based on the particular behavior or characteristics of the model. For example, data including patient outcomes or results is used to generate or develop a model (e.g., train and test the model so that the model can then be deployed for use). As such, results are used to generate a model, and the model is then used to generate probabilities associated with a particular target, focus, or question. A threshold is determined based on one or more concerns, requirements, boundaries, preferences, etc., to specify a certain combination of precision (e.g., a positive prediction value) and recall (e.g., a true positive rate or sensitivity) of the model.

By specifying a certain threshold, the model's percentage of positive predictions and its true positivity rate are acceptable for the model's purpose in that situation. That threshold can change based on circumstance (e.g., individual patient treatment, cohort treatment in a clinical trial, patient life stage, disease severity, patient preference, etc.) and/or over time (e.g., as the patient changes, disease/symptoms change, preference changes, etc.). As such, a model can have applicability to a variety of situations based on the threshold selected for output for that particular patient in that particular situation. As the patient and/or the given patient's circumstances change over time, the model can change as well.

In certain examples, a model can be generated and set to a certain state or mode (e.g., a “clinical trial mode” in which the model identifies patients suitable for this clinical trial, a “patient treatment mode” in which the model determines whether this particular treatment would likely benefit this particular patient, etc.). As such, a model can be loaded and set for a particular purpose, and that purpose can further be refined by setting a particular patient selection criterion/threshold.

FIG. 20 illustrates an example predictive output of an overall survival model. An x-axis 2010 of the graph corresponds to a threshold (e.g., a patient selection criterion), and a y-axis 2020 of the graph corresponds to a probability of survival (e.g., qualified by a precision curve 2030 or a recall curve 2040). A threshold 2010 selected for the patient selection criterion sets a point, for example, at which below the threshold, one action is taken and, above the threshold, another action is taken.

In the example of FIG. 20, patient results are reflected in the model. The precision 2030 and recall 2040 curves indicate a likelihood of true positive, false positive, or false negative result. For example, a patient's survival is a data point classified as a negative result. A patient who did not survive is a data point classified as a positive result. An associated probability is indicated on the y-axis 2020. For example, from 0.0 to 1.0 (indicating a 0% chance up to a 100% chance), a probability of greater than 0.5 indicates that the patient is more likely to not survive than to survive. A probability of less than 0.5 indicates that the patient is more likely to survive than not. A probability of 0.5 indicates a 50/50 chance of either outcome. The precision 2030 and recall 2040 curves tell whether a sampling of these data points are likely to be a true positive, a false positive, or a false negative. The precision 2030 and recall 2040 curves drive determination of a corresponding patient selection criterion threshold by which to make the model output actionable.

For example, as shown in FIG. 20, if a low threshold (e.g., 0.0 to 0.3) is selected, then the model result has low precision (e.g., many false positives) and high recall (e.g., almost no false negatives). As such, with a low threshold, there is a low likelihood of incorrectly identifying someone as alive but a high likelihood of misidentifying some as deceased. As a result, almost all patients who are predicted to be alive following the immunotherapy treatment will be alive. However, the model wrongly predicts many deaths that do not actually occur. In the pharmaceutical clinical trial use case, the model will screen out or eliminate too many patients from the trial due to the inflated death numbers. In the clinical practice use case for patient treatment, the model will potentially withhold immunotherapy from too many patients because of the inflated probability of death.

For example, if a threshold of 0.1 is selected in the example of FIG. 20, then no false survivals are predicted (no false negatives) because the recall 2040 is at 1.0. However, there is about a 50/50 chance that a predicted death is incorrect (a false positive) because the precision 2030 is around 0.55.

If an intermediate threshold is selected (e.g., 0.4 to 0.6), then there is more balance between false positives and false negatives. In the pharmaceutical use case, the model will screen out or exclude some patients who may have survived and screen in or include some patients who will not survive. In the clinical practice context, the model will make some inappropriate predictions, but most patients for whom immunotherapy is recommended will survive. With this threshold applied to the model, most patients for whom the treatment is not recommended would not have survived.

For example, if a threshold of 0.5 is selected in the example of FIG. 20, then relatively few false survivals are predicted because the recall 2040 is around 0.75. Additionally, the model also provides relatively few incorrect predictions regarding lack of survival because the precision is approximately 0.65. As such, a good balance of false positives and false negatives is provided at this threshold. Then, a patient whose probability of survival is at least 0.5 would be selected for the treatment, for example.

If a high threshold is selected (e.g., 0.7 to 1.0), then the model output has high precision (e.g., very few false positives) and low recall (e.g., many false negatives). As a result, almost all patients who are predicted to be deceased will be truly deceased, and the model wrongly predicts many survivals that do not actually occur. In the pharmaceutical context, the model will include too many patients in the clinical trial that will not survive. In the clinical patient care context, the model will incorrectly indicate immunotherapy too often.

For example, if a threshold of 0.9 is selected in the example of FIG. 20, then almost all predicted survivals are false because the recall 2040 is at 0.0. However, all predicted deaths are correct because the precision 2030 is at 1.0.

Using the precision 2030 and recall 2040 characteristics of the model represented in FIG. 20, the patient selection criterion threshold 2010 can be selected to drive clinical trial cohort selection, patient treatment determination, etc. The model, trained on a broader population of prior healthcare data, outputs a probability of survival for a given patient by processing that particular patient's healthcare data to generate a predictive output representative of efficacy (using overall survivability as a surrogate or substitute for efficacy of the immunotherapy treatment) for that patient. The model output for that patient is evaluated according to the determined threshold 2010 to determine whether or not the patient is an acceptable candidate for clinical trial cohort, immunotherapy treatment regime, etc. Based on clinical trial motivations and constraints, a patient's personal circumstances and desires, etc., the patient selection threshold 2010 can be adjusted to be more or less inclusive/exclusive to include or exclude the particular patient from the clinical trial cohort, treatment plan, etc., taking on an associated risk of inaccuracy/accuracy as the threshold is moved according to the precision 2030 and recall 2040 curves for the model.

As shown in the example of FIG. 20, selecting an intermediate threshold of 0.55 represents an intersection of the precision 2030 and recall 2040 curves at approximately 0.7. Such an intersection provides good precision and good recall with less than one-third false positives and less than one-third false negatives, resulting in the model making some inaccurate predictions, but most patients recommended for immunotherapy will survive and most patients for which the immunotherapy is not recommended would not have survived. As such, an overall patient survival rate is improved due to the characteristics of the model.

To generate the model represented in FIG. 20, data and associated features were used. Features used to train and test this model include features representing an aggregation of lab measurements over a time period (e.g., 120 days, 100 days, 30 days, etc.) as well as an aggregation of patient conditions, medications, etc., over a time period (e.g., 1 year, 9 months, 6 months, etc.), which can be generated using one hot encoding and methods such as last value, median time-weighted average (twa), mean, median, etc. Features included lymph node percentage, blood albumin (e.g., last value, twa, median, etc.), dexamethasone drug count, count of chemo-related procedures (e.g., number of chemotherapies administered in the aggregation period), red blood cell distribution width (RDW) (e.g., twa, last, median, etc.), red blood cell count (RBC), hemoglobin (twa, median, last, etc.), kidney function, lung function, cancer stage, cancer recurrence, etc. As described above, the features were combined, weighted, and iterated to generate the model, which is reflected in the example of FIG. 20. This model can be deployed and used to predict outcomes for patients.

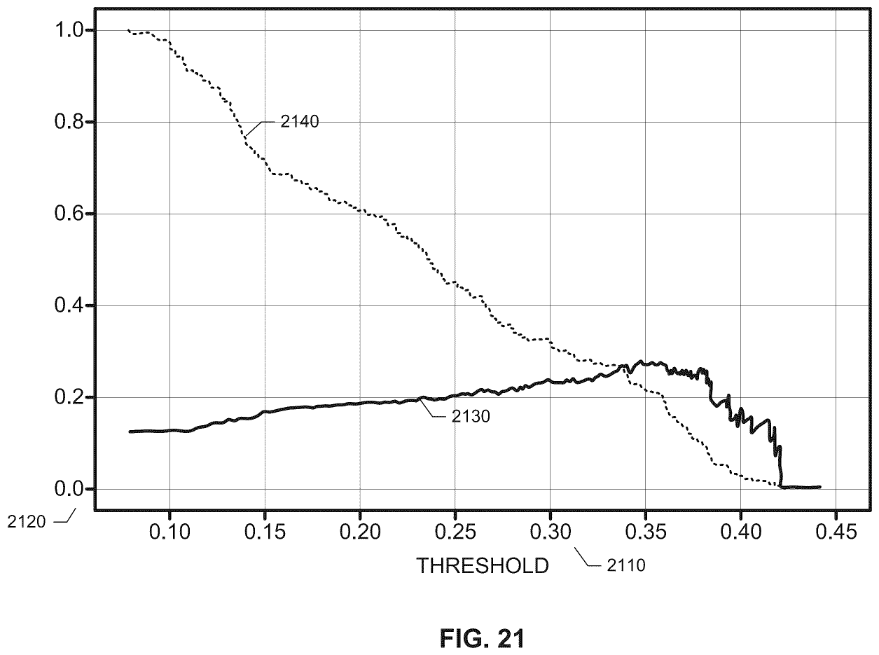

FIG. 21 illustrates an example predictive output of a pneumonitis toxicity model. An x-axis 2110 of the graph corresponds to a threshold (e.g., a patient selection criterion), and a y-axis 2120 of the graph corresponds to a probability of developing pneumonitis toxicity (e.g., on a precision curve 2130 or a recall curve 2140). A threshold 2110 selected for the patient selection criterion sets a point at which below the threshold, one action is taken and, above the threshold, another action is taken.

In the example of FIG. 21, a negative result indicates that the patient does not suffer from pneumonitis toxicity, and a positive result indicates that the patient does suffer from pneumonitis toxicity. If a low threshold (e.g., 0.0 to 0.25) is selected, then the model result has low precision (e.g., many false positives) and high recall (e.g., almost no false negatives). As a result, almost all patients who are predicted not to suffer pneumonitis toxicity as a result of taking the immunotherapy will not have pneumonitis. However, the model wrongly predicts many pneumonitis toxicities in patients that do not actually occur. In the pharmaceutical clinical trial use case, the model will screen out or eliminate too many patients from the trial due to the inflated pneumonitis toxicity numbers. In the clinical practice use case for patient treatment, the model will potentially withhold immunotherapy from too many patients because of the inflated probability of toxicity.

For example, if a threshold of 0.1 is selected in the example of FIG. 21, then almost all predictions of no toxicity are true because the recall 2140 is at almost 1.0. However, most predicted toxicities are incorrect because the precision 2130 is at a little over 0.1.

However, if an intermediate or high threshold is selected, then both precision 2130 and recall 2140 are low, providing large numbers of false positives and false negatives. As such, large numbers will be excluded from clinical trial or treatment.

For example, if a threshold of 0.3 is selected in the example of FIG. 21, then almost all predictions of no toxicity are false because the recall 2140 is at 0.3. Additionally, most predicted toxicities are incorrect because the precision 2130 is at a little over 0.2. Similarly, if a threshold of 0.45 is selected, the all predictions of no toxicity and toxicity are false because the precision 2130 and recall 2140 are both 0.0.

Using the precision 2130 and recall 2140 characteristics of the model represented in FIG. 21, the patient selection criterion threshold 2110 can be selected to drive clinical trial cohort selection, patient treatment determination, etc. The model, trained on a broader population of prior healthcare data, outputs a probability of developing pneumonitis toxicity from the immunotherapy for a given patient by processing that particular patient's healthcare data to generate a predictive output representative of pneumonitis toxicity for that patient. The model output for that patient is evaluated according to the determined threshold 2110 to determine whether or not the patient is an acceptable candidate for clinical trial cohort, immunotherapy treatment regime, etc. Based on clinical trial motivations and constraints, a patient's personal circumstances and desires, etc., the patient selection threshold 2110 can be adjusted to be more or less inclusive/exclusive to include or exclude the particular patient from the clinical trial, treatment plan, etc., taking on an associated risk of inaccuracy/accuracy as the threshold is moved according to the precision 2130 and recall 2140 curves for the model.

In the example of FIG. 21, an intersection of the precision 2130 and recall 2140 curves occurs at approximately 0.34. At this intersection point, however, both precision 2130 and recall 2140 are poor, at less than 0.3 each, indicating over 70% false positives and over 70% false negatives. As such, the intersection is good for neither precision nor recall. With the pneumonitis toxicity model of FIG. 21, precision 2130 and recall 2140 are not well aligned, and precision 2130 is poor throughout the model. Thus, a threshold 2110 can be selected based on recall 2140 at 0.7 with precision 2130 around 0.18, for example, since precision hovers around 0.2 until both precision 2130 and recall 2140 drop from 0.35 to 0.45.

As such, a desired predictability of outcome must be balanced with the reality of the model's characteristics based on the data and correlations used to generate that model. As shown in FIG. 21, there is not always a perfect balance between precision and recall on which to base an evaluation of model predictive output. As described above, the model's characteristics can change over time as the available data changes, associated features change, feedback from an old model is provided, etc.

To generate the model represented in FIG. 21, features and associated data were used. Features used to train and test this model include features representing an aggregation of lab measurements over a time period (e.g., 120 days, 100 days, 30 days, etc.) as well as an aggregation of patient conditions, medications, etc., over a time period (e.g., 1 year, 9 months, 6 months, etc.) that can be generated using one hot encoding and methods such as last value, median time-weighted average (twa), mean, median, etc. For example, data input to train the model can include, for each patient, all ICD-10 codes within a year prior to first immunotherapy treatment, oxygen saturation values from lab measurements within 120 days before first treatment, first immunotherapy treatment date, etc. Those data inputs are transformed into a set of features including frequency of lung-related ICD-10 codes (e.g., C34, C78, R91, J, R05, R06, R07, R09, etc.), frequency of ICD-10 code C34 (malignant neoplasm of bronchus and lung), frequency of ICD-10 code C78 (secondary malignant neoplasm of respiratory and digestive organs), median oxygen saturation in blood lab measurements within 120 days before first ICI treatment, smoking, etc. The features train the model (e.g., as a binary classifier using random forest, etc.) to generate a predicted probability of developing pneumonitis toxicity at a time after immunotherapy treatment. As described above, the features were combined, weighted, and iterated to generate the model, which is reflected in the example of FIG. 21. This model can be deployed and used to predict outcomes for patients.

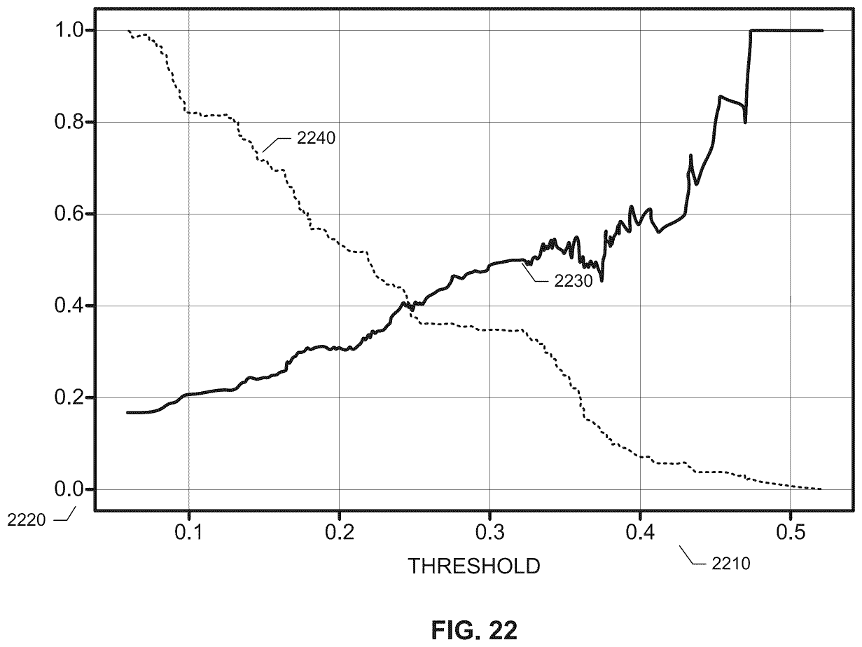

FIG. 22 illustrates an example predictive output of a colitis toxicity model. An x-axis 2210 of the graph corresponds to a threshold (e.g., a patient selection criterion), and a y-axis 2220 of the graph corresponds to a probability of developing colitis toxicity (e.g., on a precision curve 2230 or a recall curve 2240). A threshold 2210 selected for the patient selection criterion sets a point at which below the threshold, one action is taken and, above the threshold, another action is taken.

In the example of FIG. 22, a negative result indicates that the patient does not suffer from colitis toxicity, and a positive result indicates that the patient does suffer from colitis toxicity. If a low threshold (e.g., 0.0 to 0.2) is selected, then the model result has low precision (e.g., many false positives) and high recall (e.g., almost no false negatives). As a result, almost all patients who are predicted not to suffer colitis toxicity as a result of taking the immunotherapy will not have colitis. However, the model wrongly predicts many colitis toxicities in patients that do not actually occur. In the pharmaceutical clinical trial use case, the model will screen out or eliminate too many patients from the trial due to the inflated colitis toxicity numbers. In the clinical practice use case for patient treatment, the model will potentially withhold immunotherapy from too many patients because of the inflated probability of colitis toxicity.

For example, if a threshold of 0.1 is selected in the example of FIG. 22, then almost all predictions of no toxicity are true because the recall 2240 is at 0.8. However, most predicted toxicities are incorrect because the precision 2230 is at 0.2.

If an intermediate threshold is selected (e.g., 0.2 to 0.33), then there is more balance between false positives and false negatives. In the pharmaceutical use case, the model will screen out or exclude some patients who will not experience colitis toxicity and will screen in or include some patients who will experience colitis toxicity.

For example, if a threshold of 0.3 is selected in the example of FIG. 22, then less than half of the predictions of no toxicity are true because the recall 2240 is at 0.35. However, approximately half of the predicted toxicities are correct/incorrect because the precision 2230 is approximately 0.5.

If a high threshold is selected (e.g., 0.33 to 0.6), then the model output has high precision (e.g., very few false positives) and low recall (e.g., many false negatives). As a result, almost all patients who are predicted to suffer colitis toxicity will truly have colitis, and the model fails to predict many colitis toxicities that do actually occur. In the pharmaceutical context, the model will include too many patients in the clinical trial that will experience colitis toxicity. In the clinical patient care context, the model will falsely underestimate colitis toxicity occurrence.

For example, if a threshold of 0.5 is selected in the example of FIG. 22, then none of the predictions of no toxicity are true because the recall 2240 is at 0.0. However, all of the predicted toxicities are correct because the precision 2230 is at 1.0.

Using the precision 2230 and recall 2240 characteristics of the model represented in FIG. 22, the patient selection criterion threshold 2210 can be selected to drive clinical trial cohort selection, patient treatment determination, etc. The model, trained on a broader population of healthcare data, outputs a probability of developing colitis toxicity from the immunotherapy for a given patient by processing that particular patient's healthcare data to generate a predictive output representative of colitis toxicity for that patient. The model output for that patient is evaluated according to the determined threshold 2210 to determine whether or not the patient is an acceptable candidate for clinical trial cohort, immunotherapy treatment regime, etc. Based on clinical trial motivations and constraints, a patient's personal circumstances and desires, etc., the patient selection threshold 2210 can be adjusted to be more or less inclusive/exclusive to include or exclude the particular patient from the clinical trial, treatment plan, etc., taking on an associated risk of inaccuracy/accuracy as the threshold 2210 is moved according to the precision 2230 and recall 2240 curves for the model.

As shown in the example of FIG. 22, selecting a threshold of 0.25 represents an intersection of the precision 2230 and recall 2240 curves at approximately 0.4. Such an intersection provides decent precision and good recall. However, false positives and false negatives are both still over 50%. As such, a different threshold 2210 can be selected to prioritize recall 2240 (e.g., at 0.15 to reduce false negatives) or precision 2230 (e.g., at 0.35 to reduce false positives).

To generate the model represented in FIG. 22, data and associated features were used. Features used to train and test this model include features representing an aggregation of lab measurements over a time period (e.g., 120 days, 100 days, 30 days, etc.) as well as an aggregation of patient conditions, medications, etc., over a time period (e.g., 1 year, 9 months, 6 months, etc.) that can be generated using one hot encoding and methods such as last value, median time-weighted average (twa), mean, median, etc. For example, features formed to train the model can include class of immunotherapy drug, condition (e.g., one-hot encoded, etc.), below normal hemoglobin blood frequency, number of chemotherapy-related procedures in the time frame, below/above normal lymphocyte number, number of unique immunotherapy drugs, albumin below/above normal, white blood cell count (WBC) below/above normal, RBC below/above normal, hemoglobin above/below normal, immunotherapy drug class, etc. The features train the model (e.g., as a binary classifier using random forest, etc.) to generate a predicted probability of developing colitis toxicity at a time after immunotherapy treatment. As described above, the features were combined, weighted, and iterated to generate the model, which is reflected in the example of FIG. 22. This model can be deployed and used to predict outcomes for patients.

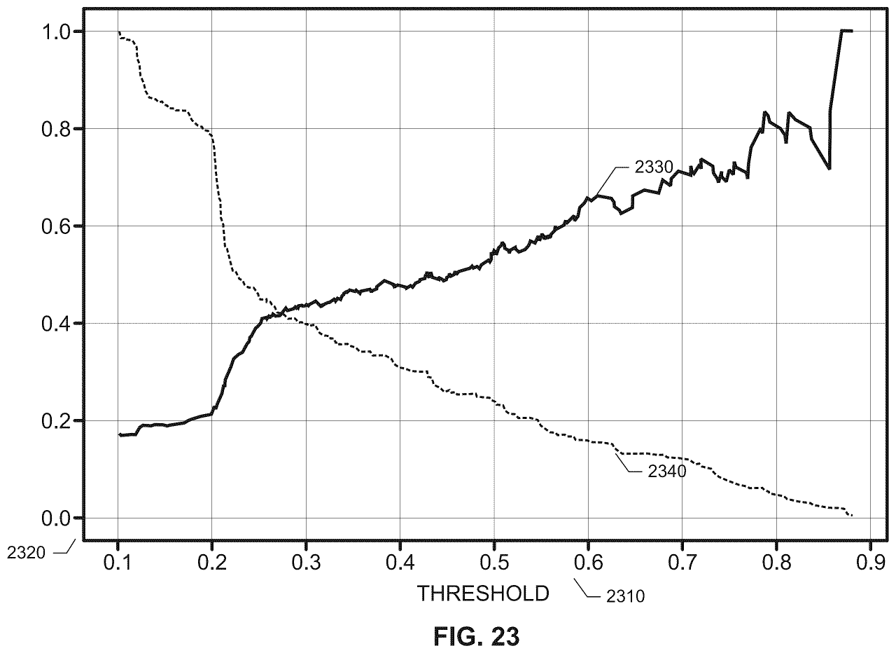

FIG. 23 illustrates an example predictive output of a hepatitis toxicity model. An x-axis 2310 of the graph corresponds to a threshold (e.g., a patient selection criterion), and a y-axis 2320 of the graph corresponds to a probability of developing hepatitis toxicity (e.g., on a precision curve 2330 or a recall curve 2340). A threshold 2310 selected for the patient selection criterion sets a point at which below the threshold, one action is taken and, above the threshold, another action is taken.

In the example of FIG. 23, a negative result indicates that the patient does not suffer from hepatitis toxicity, and a positive result indicates that the patient does suffer from hepatitis toxicity. If a low threshold (e.g., 0.0 to 0.2) is selected, then the model result has low precision (e.g., many false positives) and high recall (e.g., almost no false negatives). As a result, the model wrongly predicts many hepatitis toxicities in patients that do not actually occur, but few patients who are predicted not to suffer hepatitis toxicity as a result of taking the immunotherapy will actually have hepatitis.

For example, if a threshold of 0.1 is selected in the example of FIG. 23, then all predictions of no toxicity are true because the recall 2340 is at 1.0. However, most of the predicted toxicities are incorrect because the precision 2330 is at slightly less than 0.2.

If an intermediate threshold is selected (e.g., 0.2 to 0.6), then there is more balance between false positives and false negatives. In the pharmaceutical use case, the model will screen out or exclude some patients who will not experience hepatitis toxicity and will screen in or include some patients who will experience hepatitis toxicity.

For example, if a threshold of 0.5 is selected in the example of FIG. 23, then most predictions of no toxicity are incorrect because the recall 2340 is at 0.25. However, approximately half of the predicted toxicities are correct because the precision 2330 is at 0.55.

If a high threshold is selected (e.g., 0.6 to 1.0), then the model output has high precision (e.g., few false positives) and low recall (e.g., many false negatives). As a result, many patients who are predicted to suffer from hepatitis toxicity will actually have hepatitis, but patients who are predicted not to suffer from hepatitis toxicity will have hepatitis. In the pharmaceutical context, the model will include too many patients in the clinical trial that will experience hepatitis toxicity. In the clinical patient care context, the model will falsely underestimate hepatitis toxicity occurrence.

For example, if a threshold of 0.9 is selected in the example of FIG. 23, then no predictions of no toxicity are true because the recall 2340 is 0.0. However, all of the predicted toxicities are correct because the precision 2330 is 1.0.

Using the precision 2330 and recall 2340 characteristics of the model represented in FIG. 23, the patient selection criterion threshold 2310 can be selected to drive clinical trial cohort selection, patient treatment determination, etc. The model, trained on a broader population of prior healthcare data, outputs a probability of developing hepatitis toxicity from the immunotherapy for a given patient by processing that particular patient's healthcare data to generate a predictive output representative of hepatitis toxicity for that patient. The model output for that patient is evaluated according to the determined threshold 2310 to determine whether or not the patient is an acceptable candidate for clinical trial cohort, immunotherapy treatment regime, etc. Based on clinical trial motivations and constraints, a patient's personal circumstances and desires, etc., the patient selection threshold 2310 can be adjusted to be more or less inclusive/exclusive to include or exclude the particular patient from the clinical trial, treatment plan, etc., taking on an associated risk of inaccuracy/accuracy as the threshold 2310 is moved according to the precision 2330 and recall 2340 curves for the model.

As shown in the example of FIG. 23, an intersection of the precision 2330 and recall 2340 curves occurs at approximately 0.28. At this intersection point, however, both precision 2330 and recall 2340 are slightly less than average, at a little more than 0.4 each, indicating approximately 60% false positives and approximately 60% false negatives. As such, the intersection is not great for either precision or recall. Thus, a threshold 2310 can be selected based on recall 2340 at 1.0 with precision 2330 around 0.19, for example, or based on recall 2340 at 0.58 with precision 2330 at 0.25, for example, depending on the selection criterion for the model, patient, etc.

To generate the model represented in FIG. 23, data and associated features were used. Features used to train and test this model include features representing an aggregation of lab measurements over a time period (e.g., 120 days, 100 days, 30 days, etc.) as well as an aggregation of patient conditions, medications, etc., over a time period (e.g., 1 year, 9 months, 6 months, etc.) that can be generated using one hot encoding and methods such as last value, median time-weighted average (twa), mean, median, etc. For example, features formed to train the model can include level of alkaline phosphatase in blood (e.g., last, twa, max, min, etc.), aspartate aminotransferase in blood (e.g., last, twa, max, min, etc.), level of alanine transaminase in blood (e.g., last, twa, max, min, etc.), bilirubin in blood (e.g., max, twa, last, min, etc.), etc. The features train the model (e.g., a binary classifier using random forest) to generate a predicted probability of developing hepatitis toxicity at a time after immunotherapy treatment. As described above, the features were combined, weighted, and iterated to generate the model, which is reflected in the example of FIG. 23. This model can be deployed and used to predict outcomes for patients.

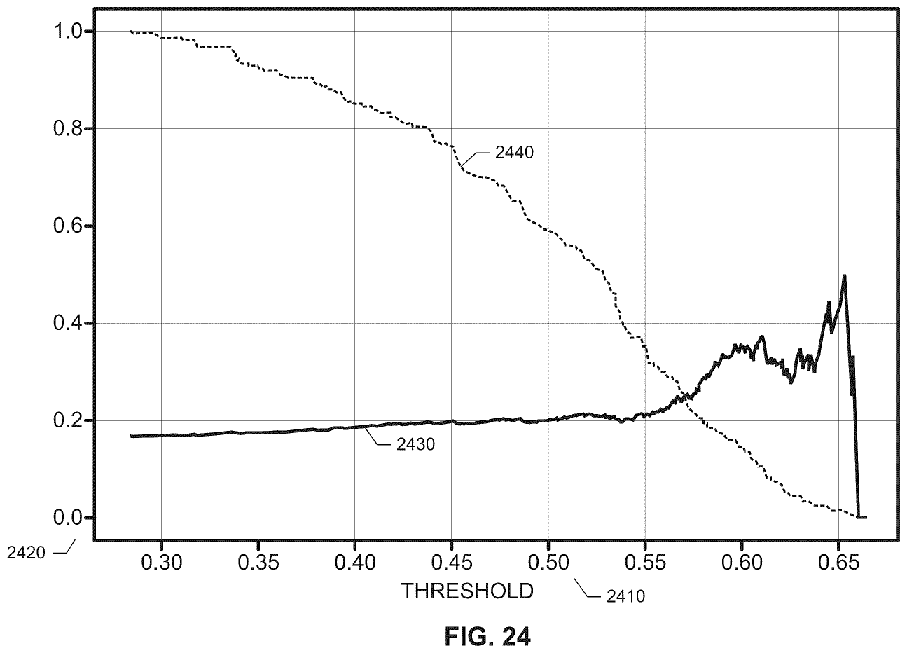

FIG. 24 shows characteristics of a time on treatment model, which is another surrogate for treatment efficacy that can be used instead of or in addition to the overall survivability model reflected in FIG. 20. An x-axis 2410 of the graph corresponds to a threshold (e.g., a patient selection criterion), and a y-axis 2420 of the graph corresponds to a probability of remaining on the immunotherapy treatment (e.g., on a precision curve 2430 or a recall curve 2440). A threshold 2410 selected for the patient selection criterion sets a point at which below the threshold, one action is taken and, above the threshold, another action is taken.

In the example of FIG. 24, a negative result indicates that the patient continues treatment, and a positive result indicates that the patient has stopped treatment. If a low threshold (e.g., 0.0 to 0.5) is selected, then the model result has high precision (e.g., very few false positives) and low recall (e.g., many false negatives). As such, there is a high likelihood of incorrectly identifying someone who has stopped treatment but a low likelihood of misidentifying some who is still on treatment. As a result, almost all patients who are predicted to still be taking the immunotherapy treatment will in fact be taking the immunotherapy. However, the model wrongly predicts many cessations of treatment that do not actually occur. In the pharmaceutical clinical trial use case, the model will screen out or eliminate too many patients from the trial due to the inflated treatment cessation numbers. In the clinical practice use case for patient treatment, the model will potentially withhold immunotherapy from too many patients because of the inflated probability that the immunotherapy will have to prematurely cease.

For example, if a threshold of 0.3 is selected in the example of FIG. 24, then all predictions of stopped treatment are true because the recall 2440 is almost 1.0. However, most of the predictions of continued treatment are incorrect because the precision 2430 is slightly less than 0.2.

If an intermediate threshold is selected (e.g., 0.5 to 0.6), then there is more balance between false positives and false negatives. In the pharmaceutical use case, the model will screen out or exclude some patients who may have completed the treatment and screen in or include some patients who will not continue with the treatment. However, an overall survival rate is improved due to the characteristics of the model. In the clinical practice context, the model will make some inappropriate predictions, but most patients for whom immunotherapy is recommended will complete the treatment. With this threshold applied to the model, most patients for whom the treatment is not recommended would not have been able to complete the treatment.

For example, if a threshold of 0.5 is selected in the example of FIG. 24, then more than half of the predictions of stopped treatment are true because the recall 2440 is 0.6. However, most of the predictions of continued treatment are incorrect because the precision 2430 is 0.2.

If a high threshold is selected (e.g., 0.6 to 0.7), then the model output has low precision (e.g., many false positives) and high recall (e.g., few false negatives). As a result, almost all patients who are predicted to not complete the treatment will in fact not complete the treatment, and the model wrongly predicts many ceased treatment regimens that are actually still occurring. In the pharmaceutical context, the model will exclude too many patients in the clinical trial that would complete the treatment regimen. In the clinical patient care context, the model will incorrectly indicate immunotherapy too rarely.

For example, if a threshold of 0.65 is selected in the example of FIG. 24, then almost all predictions of stopped treatment are untrue because the recall 2440 is slightly above 0.0. Additionally, most of the predictions of continued treatment are incorrect because the precision 2430 is slightly greater than 0.4.

Using the precision 2430 and recall 2440 characteristics of the model represented in FIG. 24, the patient selection criterion threshold 2410 can be selected to drive clinical trial cohort selection, patient treatment determination, etc. The model, trained on a broader population of prior healthcare data, outputs a probability of successful treatment for a given patient by processing that particular patient's healthcare data to generate a predictive output representative of efficacy (using time on treatment as a surrogate or substitute for efficacy of the immunotherapy treatment) for that patient. The model output for that patient is evaluated according to the determined threshold 2410 to determine whether or not the patient is an acceptable candidate for clinical trial cohort, immunotherapy treatment regime, etc. Based on clinical trial motivations and constraints, a patient's personal circumstances and desires, etc., the patient selection threshold 2410 can be adjusted to be more or less inclusive/exclusive to include or exclude the particular patient from the clinical trial, treatment plan, etc., taking on an associated risk of inaccuracy/accuracy as the threshold 2410 is moved according to the precision 2430 and recall 2440 curves for the model.

As shown in the example of FIG. 24, an intersection of the precision 2430 and recall 2440 curves occurs at approximately 0.57. At this intersection point, however, both precision 2430 and recall 2440 are poor, at less than 0.3 each, indicating over 70% false positives and over 70% false negatives. As such, the intersection is good for neither precision nor recall. With the time-on-treatment model of FIG. 24, precision 2430 and recall 2440 are not well aligned, and precision 2430 is poor throughout the model. Thus, a threshold 2410 can be selected to maximize recall 2440 at 1.0, for example, since precision hovers around 20% unless the recall is 0.0.

To generate the model represented in FIG. 24, data and associated features were used. Features used to train and test this model include features representing an aggregation of lab measurements over a time period (e.g., 120 days, 100 days, 30 days, etc.) as well as an aggregation of patient conditions, medications, etc., over a time period (e.g., 1 year, 9 months, 6 months, etc.) that can be generated using one hot encoding and methods such as last value, median time-weighted average (twa), mean, median, etc. Features can include RBC (e.g., median, etc.), promethazine drug count, immgranulocytes (e.g., median, etc.), lymphocytes (e.g., median, etc.), blood albumin (e.g., last value, twa, median, etc.), RDW (e.g., median, etc.), neutrophils (e.g., median), alkanine phosphatase (e.g., median, etc.), dexamethasone drug count, neutabs (e.g., median, etc.), radiology count, one-hot encoded condition (e.g., for codes C77, R05, G89, etc.), chemotherapies in a time window, drug counts, etc. As described above, the features were combined, weighted, and iterated to generate the model, which is reflected in the example of FIG. 24. This model can be deployed and used to predict outcomes for patients.

As such, precision and recall can vary from model to model, and a given model can change over time as more data is gathered, the patient population changes, the particular patient changes, feedback is provided to regenerate or otherwise update the model, etc. However, the model framework enables the generation of a variety of models and the dynamic setting of threshold(s) and other patient and/or model selection criterion to drive productive, reliable outcomes for patient treatment, including by the patient's physician, as part of a clinical trial, etc.

As described herein, configuring a cohort for a clinical trial and configuring a treatment plan are two examples of how the developed models can be used. However, a patient's treatment plan could be or include to place that patient in a clinical trial (e.g., to see if the immunotherapy in the clinical trial helps the patient). As such, configuring a cohort for a clinical trial could be a particular instance of or environment for configuring a patient treatment plan to involve immunotherapy.

In some examples, decisions can be driven by a toxicity-related model alone. For example, risk of toxicity, such as pneumonitis, hepatitis, or colitis, can be the driving factor as to whether or not a patient is given immunotherapy (e.g., either as part of a clinical trial or as part of an individual treatment plan). For example, a patient may want treatment at all costs. In other examples, likelihood of treatment efficacy can be the driver of whether to administer an immunotherapy to the patient, regardless of the risk of toxicity. For example, a patient may prioritize quality of life over more aggressive treatment. In such instances, additional tests and monitoring (e.g., check-ins, model re-runs, etc.) may be ordered to help ensure patient health should toxicity start to develop. In some examples, output of both an efficacy-related model and one or more toxicity-related models can be combined to balance likelihood of success of the immunotherapy with chance of developing one or more toxicities. As such, a model can provide a binary output (e.g., include/exclude, treat/do not treat, etc.) and/or a multi-faceted or nuanced output that conveys more than a yes or no answer, for example.

As such, the model framework can generate a plurality of models that are used in various ways, according to various thresholds, based on clinical needs, study needs, patient desires, etc. Such qualitative indicators can be transformed into quantitative thresholds for patient selection criterion, which enable the same model to drive different decisions and behaviors according to the threshold. Patient selection criterion can change over time, as a patient may initially want to be aggressive, then pull back to allow them to go on vacation, then resume more aggressively, and possibly pull back again as end of life quality decisions are made. The models can accommodate all of these circumstances through the setting of the patient selection criterion thresholds (as well as through the model selection criterion that drives model generation for a certain purpose or focus), for example.

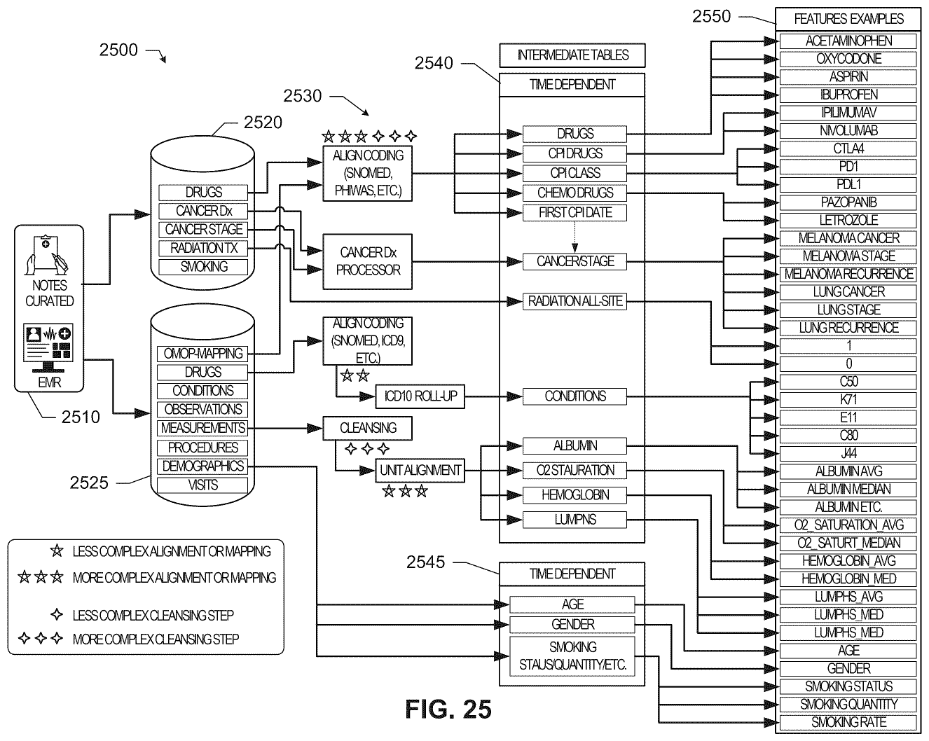

FIG. 25 illustrates an example infrastructure and associated process 2500 by which patient healthcare data from one or more patient healthcare records 2510 is organized/curated in one or more data stores 2520, 2525 for intermediate processing using one or more alignment, mapping, and/or cleansing actions 2530 to form time-dependent values 2540, 2545. The time-dependent values from the intermediate tables form features 2550 that can be used for model development, as described above. Thus, healthcare data from existing records 2510 is pulled in and organized 2520 for processing 2530 according to varying degrees of complexity. The intermediate tables 2540, 2545 form time-series data values or variables that correlate to multiple features 2550. The features can be used to train and test multiple models, such as described above.

While example implementations are illustrated in this application and associated appendix, one or more of the elements, processes, and/or devices illustrated may be combined, divided, re-arranged, omitted, eliminated, and/or implemented in any other way. Further, the example elements may be implemented by hardware alone or by hardware in combination with software and/or firmware. Thus, for example, any of the example elements could be implemented by processor circuitry, analog circuit(s), digital circuit(s), logic circuit(s), programmable processor(s), programmable microcontroller(s), graphics processing unit(s) (GPU(s)), digital signal processor(s) (DSP(s)), application specific integrated circuit(s) (ASIC(s)), programmable logic device(s) (PLD(s)), and/or field programmable logic device(s) (FPLD(s)) such as Field Programmable Gate Arrays (FPGAs). Further still, the example elements may include one or more elements, processes, and/or devices in addition to, or instead of, those illustrated, and/or may include more than one of any or all of the illustrated elements, processes and devices.

In certain examples, hardware logic circuitry, machine readable instructions, hardware implemented state machines, and/or any combination thereof can implement the system(s) and/or execute the methods disclosed herein. The machine readable instructions may be one or more executable programs or portion(s) of an executable program for execution by processor circuitry. The program may be embodied in software stored on one or more non-transitory computer readable storage media such as a compact disk (CD), a floppy disk, a hard disk drive (HDD), a solid-state drive (SSD), a digital versatile disk (DVD), a Blu-ray disk, a volatile memory (e.g., Random Access Memory (RAM) of any type, etc.), or a non-volatile memory (e.g., electrically erasable programmable read-only memory (EEPROM), FLASH memory, an HDD, an SSD, etc.) associated with processor circuitry located in one or more hardware devices, but the entire program and/or parts thereof could alternatively be executed by one or more hardware devices other than the processor circuitry and/or embodied in firmware or dedicated hardware. The machine readable instructions may be distributed across multiple hardware devices and/or executed by two or more hardware devices (e.g., a server and a client hardware device). For example, the client hardware device may be implemented by an endpoint client hardware device (e.g., a hardware device associated with a user) or an intermediate client hardware device (e.g., a radio access network (RAN)) gateway that may facilitate communication between a server and an endpoint client hardware device). Similarly, the non-transitory computer readable storage media may include one or more mediums located in one or more hardware devices. Further, an order of execution may be changed, and/or some of the blocks described may be changed, eliminated, or combined. Additionally or alternatively, any or all code blocks may be implemented by one or more hardware circuits (e.g., processor circuitry, discrete and/or integrated analog and/or digital circuitry, an FPGA, an ASIC, a comparator, an operational-amplifier (op-amp), a logic circuit, etc.) structured to perform the corresponding operation without executing software or firmware. The processor circuitry may be distributed in different network locations and/or local to one or more hardware devices (e.g., a single-core processor (e.g., a single core central processor unit (CPU)), a multi-core processor (e.g., a multi-core CPU, an XPU, etc.) in a single machine, multiple processors distributed across multiple servers of a server rack, multiple processors distributed across one or more server racks, a CPU and/or a FPGA located in the same package (e.g., the same integrated circuit (IC) package or in two or more separate housings, etc.).

The machine readable instructions described herein may be stored in one or more of a compressed format, an encrypted format, a fragmented format, a compiled format, an executable format, a packaged format, etc. Machine readable instructions as described herein may be stored as data or a data structure (e.g., as portions of instructions, code, representations of code, etc.) that may be utilized to create, manufacture, and/or produce machine executable instructions. For example, the machine readable instructions may be fragmented and stored on one or more storage devices and/or computing devices (e.g., servers) located at the same or different locations of a network or collection of networks (e.g., in the cloud, in edge devices, etc.). The machine readable instructions may require one or more of installation, modification, adaptation, updating, combining, supplementing, configuring, decryption, decompression, unpacking, distribution, reassignment, compilation, etc., in order to make them directly readable, interpretable, and/or executable by a computing device and/or other machine. For example, the machine readable instructions may be stored in multiple parts, which are individually compressed, encrypted, and/or stored on separate computing devices, wherein the parts when decrypted, decompressed, and/or combined form a set of machine executable instructions that implement one or more operations that may together form a program such as that described herein.

In another example, the machine readable instructions may be stored in a state in which they may be read by processor circuitry, but require addition of a library (e.g., a dynamic link library (DLL)), a software development kit (SDK), an application programming interface (API), etc., in order to execute the machine readable instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, machine readable media, as used herein, may include machine readable instructions and/or program(s) regardless of the particular format or state of the machine readable instructions and/or program(s) when stored or otherwise at rest or in transit.

The machine readable instructions described herein can be represented by any past, present, or future instruction language, scripting language, programming language, etc. For example, the machine readable instructions may be represented using any of the following languages: C, C++, Java, C#, Perl, Python, JavaScript, HyperText Markup Language (HTML), Structured Query Language (SQL), Swift, etc.

As mentioned above, the example operations disclosed herein may be implemented using executable instructions (e.g., computer and/or machine readable instructions) stored on one or more non-transitory computer and/or machine readable media such as optical storage devices, magnetic storage devices, an HDD, a flash memory, a read-only memory (ROM), a CD, a DVD, a cache, a RAM of any type, a register, and/or any other storage device or storage disk in which information is stored for any duration (e.g., for extended time periods, permanently, for brief instances, for temporarily buffering, and/or for caching of the information). As used herein, the terms non-transitory computer readable medium, non-transitory computer readable storage medium, non-transitory machine readable medium, and non-transitory machine readable storage medium are expressly defined to include any type of computer readable storage device and/or storage disk and to exclude propagating signals and to exclude transmission media. As used herein, the terms “computer readable storage device” and “machine readable storage device” are defined to include any physical (mechanical and/or electrical) structure to store information, but to exclude propagating signals and to exclude transmission media. Examples of computer readable storage devices and machine readable storage devices include random access memory of any type, read only memory of any type, solid state memory, flash memory, optical discs, magnetic disks, disk drives, and/or redundant array of independent disks (RAID) systems. As used herein, the term “device” refers to physical structure such as mechanical and/or electrical equipment, hardware, and/or circuitry that may or may not be configured by computer readable instructions, machine readable instructions, etc., and/or manufactured to execute computer readable instructions, machine readable instructions, etc.

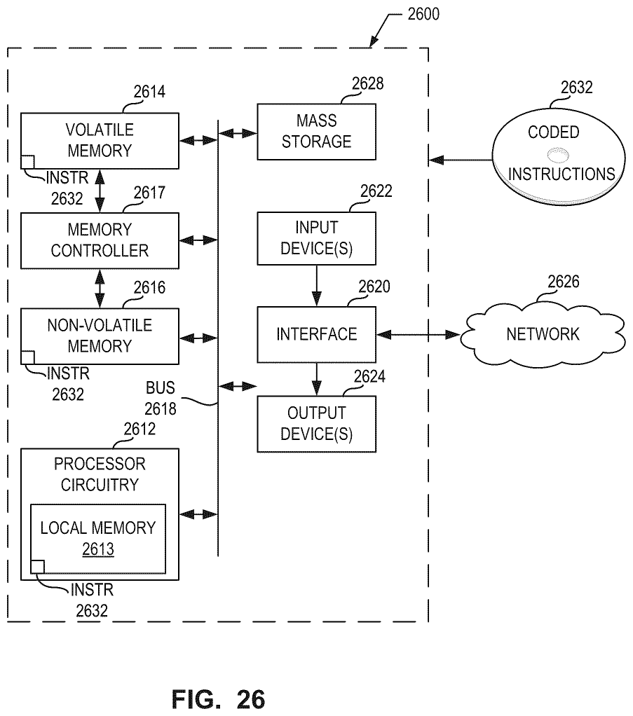

FIG. 26 is a block diagram of an example processor platform 2600 structured to execute and/or instantiate the machine readable instructions and/or the operations disclosed and described herein. The processor platform 2600 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, a DVD player, a CD player, a digital video recorder, a Blu-ray player, a gaming console, a personal video recorder, a set top box, a headset (e.g., an augmented reality (AR) headset, a virtual reality (VR) headset, etc.) or other wearable device, or any other type of computing device.

The processor platform 2600 of the illustrated example includes processor circuitry 2612. The processor circuitry 2612 of the illustrated example is hardware. For example, the processor circuitry 2612 can be implemented by one or more integrated circuits, logic circuits, FPGAs, microprocessors, CPUs, GPUs, DSPs, and/or microcontrollers from any desired family or manufacturer. The processor circuitry 2612 may be implemented by one or more semiconductor based (e.g., silicon based) devices.

The processor circuitry 2612 of the illustrated example includes a local memory 2613 (e.g., a cache, registers, etc.). The processor circuitry 2612 of the illustrated example is in communication with a main memory including a volatile memory 2614 and a non-volatile memory 2616 by a bus 2618. The volatile memory 2614 may be implemented by Synchronous Dynamic Random Access Memory (SDRAM), Dynamic Random Access Memory (DRAM), RAMBUS® Dynamic Random Access Memory (RDRAM®), and/or any other type of RAM device. The non-volatile memory 2616 may be implemented by flash memory and/or any other desired type of memory device. Access to the main memory 2614, 2616 of the illustrated example is controlled by a memory controller 2617.

The processor platform 2600 of the illustrated example also includes interface circuitry 2620. The interface circuitry 2620 may be implemented by hardware in accordance with any type of interface standard, such as an Ethernet interface, a universal serial bus (USB) interface, a Bluetooth® interface, a near field communication (NFC) interface, a Peripheral Component Interconnect (PCI) interface, and/or a Peripheral Component Interconnect Express (PCIe) interface.

In the illustrated example, one or more input devices 2622 are connected to the interface circuitry 2620. The input device(s) 2622 permit(s) a user to enter data and/or commands into the processor circuitry 2612. The input device(s) 2622 can be implemented by, for example, an audio sensor, a microphone, a camera (still or video), a keyboard, a button, a mouse, a touchscreen, a track-pad, a trackball, an isopoint device, and/or a voice recognition system.

One or more output devices 2624 are also connected to the interface circuitry 2620 of the illustrated example. The output device(s) 2624 can be implemented, for example, by display devices (e.g., a light emitting diode (LED), an organic light emitting diode (OLED), a liquid crystal display (LCD), a cathode ray tube (CRT) display, an in-place switching (IPS) display, a touchscreen, etc.), a tactile output device, a printer, and/or speaker. The interface circuitry 2620 of the illustrated example, thus, typically includes a graphics driver card, a graphics driver chip, and/or graphics processor circuitry such as a GPU.

The interface circuitry 2620 of the illustrated example also includes a communication device such as a transmitter, a receiver, a transceiver, a modem, a residential gateway, a wireless access point, and/or a network interface to facilitate exchange of data with external machines (e.g., computing devices of any kind) by a network 2626. The communication can be by, for example, an Ethernet connection, a digital subscriber line (DSL) connection, a telephone line connection, a coaxial cable system, a satellite system, a line-of-site wireless system, a cellular telephone system, an optical connection, etc.

The processor platform 2600 of the illustrated example also includes one or more mass storage devices 2628 to store software and/or data. Examples of such mass storage devices 2628 include magnetic storage devices, optical storage devices, floppy disk drives, HDDs, CDs, Blu-ray disk drives, redundant array of independent disks (RAID) systems, solid state storage devices such as flash memory devices and/or SSDs, and DVD drives.

The machine readable instructions 2632 may be stored in the mass storage device 2628, in the volatile memory 2614, in the non-volatile memory 2616, and/or on a removable non-transitory computer readable storage medium such as a CD or DVD.

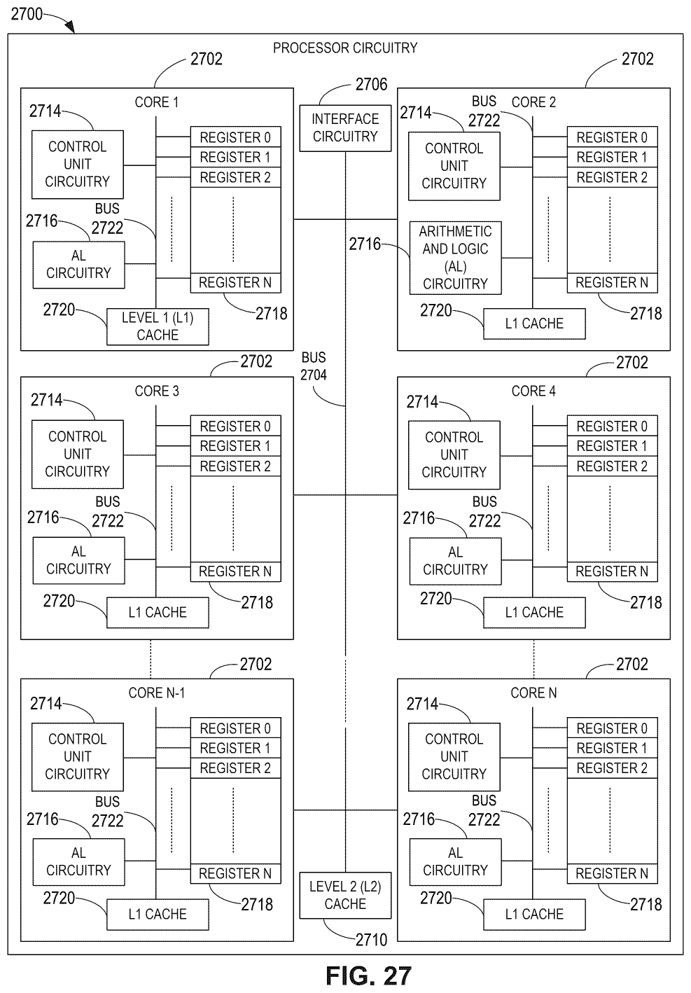

FIG. 27 is a block diagram of an example implementation of the processor circuitry 2612 of FIG. 26. In this example, the processor circuitry 2612 of FIG. 26 is implemented by a microprocessor 2700. For example, the microprocessor 2700 may be a general purpose microprocessor (e.g., general purpose microprocessor circuitry). The microprocessor 2700 executes some or all of the machine readable instructions to effectively instantiate the circuitry described herein as logic circuits to perform the operations corresponding to those machine readable instructions. In some such examples, the circuitry is instantiated by the hardware circuits of the microprocessor 2700 in combination with the instructions. For example, the microprocessor 2700 may be implemented by multi-core hardware circuitry such as a CPU, a DSP, a GPU, an XPU, etc. Although it may include any number of example cores 2702 (e.g., 1 core), the microprocessor 2700 of this example is a multi-core semiconductor device including N cores. The cores 2702 of the microprocessor 2700 may operate independently or may cooperate to execute machine readable instructions. For example, machine code corresponding to a firmware program, an embedded software program, or a software program may be executed by one of the cores 2702 or may be executed by multiple ones of the cores 2702 at the same or different times. In some examples, the machine code corresponding to the firmware program, the embedded software program, or the software program is split into threads and executed in parallel by two or more of the cores 2702. The software program may correspond to a portion or all of the machine readable instructions and/or operations disclosed herein.

The cores 2702 may communicate by a first example bus 2704. In some examples, the first bus 2704 may be implemented by a communication bus to effectuate communication associated with one(s) of the cores 2702. For example, the first bus 2704 may be implemented by at least one of an Inter-Integrated Circuit (I2C) bus, a Serial Peripheral Interface (SPI) bus, a PCI bus, or a PCIe bus. Additionally or alternatively, the first bus 2704 may be implemented by any other type of computing or electrical bus. The cores 2702 may obtain data, instructions, and/or signals from one or more external devices by example interface circuitry 2706. The cores 2702 may output data, instructions, and/or signals to the one or more external devices by the interface circuitry 2706. Although the cores 2702 of this example include example local memory 2720 (e.g., Level 1 (L1) cache that may be split into an L1 data cache and an L1 instruction cache), the microprocessor 2700 also includes example shared memory 2710 that may be shared by the cores (e.g., Level 2 (L2 cache)) for high-speed access to data and/or instructions. Data and/or instructions may be transferred (e.g., shared) by writing to and/or reading from the shared memory 2710. The local memory 2720 of each of the cores 2702 and the shared memory 2710 may be part of a hierarchy of storage devices including multiple levels of cache memory and the main memory (e.g., the main memory 2614, 2616 of FIG. 26). Typically, higher levels of memory in the hierarchy exhibit lower access time and have smaller storage capacity than lower levels of memory. Changes in the various levels of the cache hierarchy are managed (e.g., coordinated) by a cache coherency policy.

Each core 2702 may be referred to as a CPU, DSP, GPU, etc., or any other type of hardware circuitry. Each core 2702 includes control unit circuitry 2714, arithmetic and logic (AL) circuitry (sometimes referred to as an ALU) 2716, a plurality of registers 2718, the local memory 2720, and a second example bus 2722. Other structures may be present. For example, each core 2702 may include vector unit circuitry, single instruction multiple data (SIMD) unit circuitry, load/store unit (LSU) circuitry, branch/jump unit circuitry, floating-point unit (FPU) circuitry, etc. The control unit circuitry 2714 includes semiconductor-based circuits structured to control (e.g., coordinate) data movement within the corresponding core 2702. The AL circuitry 2716 includes semiconductor-based circuits structured to perform one or more mathematic and/or logic operations on the data within the corresponding core 2702. The AL circuitry 2716 of some examples performs integer based operations. In other examples, the AL circuitry 2716 also performs floating point operations. In yet other examples, the AL circuitry 2716 may include first AL circuitry that performs integer based operations and second AL circuitry that performs floating point operations. In some examples, the AL circuitry 2716 may be referred to as an Arithmetic Logic Unit (ALU). The registers 2718 are semiconductor-based structures to store data and/or instructions such as results of one or more of the operations performed by the AL circuitry 2716 of the corresponding core 2702. For example, the registers 2718 may include vector register(s), SIMD register(s), general purpose register(s), flag register(s), segment register(s), machine specific register(s), instruction pointer register(s), control register(s), debug register(s), memory management register(s), machine check register(s), etc. The registers 2718 may be arranged in a bank as shown in FIG. 27. Alternatively, the registers 2718 may be organized in any other arrangement, format, or structure including distributed throughout the core 2702 to shorten access time. The second bus 2722 may be implemented by at least one of an I2C bus, a SPI bus, a PCI bus, or a PCIe bus.

Each core 2702 and/or, more generally, the microprocessor 2700 may include additional and/or alternate structures to those shown and described above. For example, one or more clock circuits, one or more power supplies, one or more power gates, one or more cache home agents (CHAs), one or more converged/common mesh stops (CMSs), one or more shifters (e.g., barrel shifter(s)) and/or other circuitry may be present. The microprocessor 2700 is a semiconductor device fabricated to include many transistors interconnected to implement the structures described above in one or more integrated circuits (ICs) contained in one or more packages. The processor circuitry may include and/or cooperate with one or more accelerators. In some examples, accelerators are implemented by logic circuitry to perform certain tasks more quickly and/or efficiently than can be done by a general purpose processor. Examples of accelerators include ASICs and FPGAs such as those discussed herein. A GPU or other programmable device can also be an accelerator. Accelerators may be on-board the processor circuitry, in the same chip package as the processor circuitry and/or in one or more separate packages from the processor circuitry.

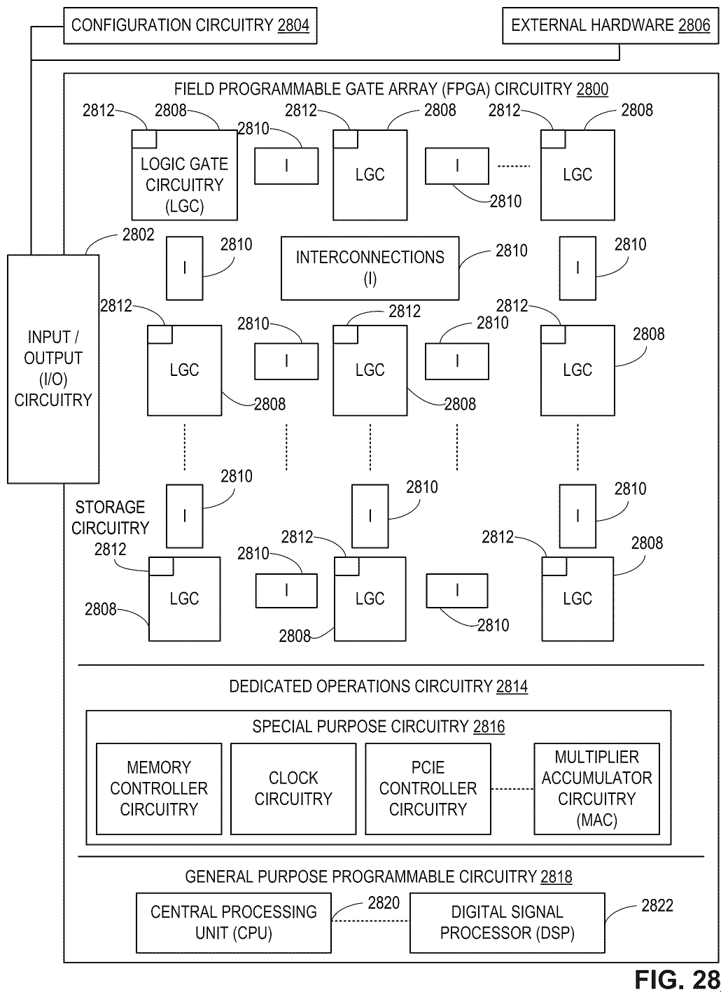

FIG. 28 is a block diagram of another example implementation of the processor circuitry 2612 of FIG. 26. In this example, the processor circuitry 2612 is implemented by FPGA circuitry 2800. For example, the FPGA circuitry 2800 may be implemented by an FPGA. The FPGA circuitry 2800 can be used, for example, to perform operations that could otherwise be performed by the example microprocessor 2700 of FIG. 27 executing corresponding machine readable instructions. However, once configured, the FPGA circuitry 2800 instantiates the machine readable instructions in hardware and, thus, can often execute the operations faster than they could be performed by a general purpose microprocessor executing the corresponding software.

More specifically, in contrast to the microprocessor 2700 of FIG. 27 described above (which is a general purpose device that may be programmed to execute some or all of the machine readable instructions disclosed here but whose interconnections and logic circuitry are fixed once fabricated), the FPGA circuitry 2800 of the example of FIG. 28 includes interconnections and logic circuitry that may be configured and/or interconnected in different ways after fabrication to instantiate, for example, some or all of the machine readable instructions disclosed herein. In particular, the FPGA circuitry 2800 may be thought of as an array of logic gates, interconnections, and switches. The switches can be programmed to change how the logic gates are interconnected by the interconnections, effectively forming one or more dedicated logic circuits (unless and until the FPGA circuitry 2800 is reprogrammed). The configured logic circuits enable the logic gates to cooperate in different ways to perform different operations on data received by input circuitry. Those operations may correspond to some or all of the software disclosed herein As such, the FPGA circuitry 2800 may be structured to effectively instantiate some or all of the machine readable instructions as dedicated logic circuits to perform the operations corresponding to those software instructions in a dedicated manner analogous to an ASIC. Therefore, the FPGA circuitry 2800 may perform the operations corresponding to the some or all of the machine readable instructions faster than the general purpose microprocessor can execute the same.

In the example of FIG. 28, the FPGA circuitry 2800 is structured to be programmed (and/or reprogrammed one or more times) by an end user by a hardware description language (HDL) such as Verilog. The FPGA circuitry 2800 of FIG. 28, includes example input/output (I/O) circuitry 2802 to obtain and/or output data to/from example configuration circuitry 2804 and/or external hardware 2806. For example, the configuration circuitry 2804 may be implemented by interface circuitry that may obtain machine readable instructions to configure the FPGA circuitry 2800, or portion(s) thereof. In some such examples, the configuration circuitry 2804 may obtain the machine readable instructions from a user, a machine (e.g., hardware circuitry (e.g., programmed or dedicated circuitry) that may implement an Artificial Intelligence/Machine Learning (AI/ML) model to generate the instructions), etc. In some examples, the external hardware 2806 may be implemented by external hardware circuitry. For example, the external hardware 2806 may be implemented by the microprocessor 2700 of FIG. 27. The FPGA circuitry 2800 also includes an array of example logic gate circuitry 2808, a plurality of example configurable interconnections 2810, and example storage circuitry 2812. The logic gate circuitry 2808 and the configurable interconnections 2810 are configurable to instantiate one or more operations that may correspond to at least some of the machine readable instructions and/or other desired operations. The logic gate circuitry 2808 shown in FIG. 28 is fabricated in groups or blocks. Each block includes semiconductor-based electrical structures that may be configured into logic circuits. In some examples, the electrical structures include logic gates (e.g., And gates, Or gates, Nor gates, etc.) that provide basic building blocks for logic circuits. Electrically controllable switches (e.g., transistors) are present within each of the logic gate circuitry 2808 to enable configuration of the electrical structures and/or the logic gates to form circuits to perform desired operations. The logic gate circuitry 2808 may include other electrical structures such as look-up tables (LUTs), registers (e.g., flip-flops or latches), multiplexers, etc.

The configurable interconnections 2810 of the illustrated example are conductive pathways, traces, vias, or the like that may include electrically controllable switches (e.g., transistors) whose state can be changed by programming (e.g., using an HDL instruction language) to activate or deactivate one or more connections between one or more of the logic gate circuitry 2808 to program desired logic circuits.

The storage circuitry 2812 of the illustrated example is structured to store result(s) of the one or more of the operations performed by corresponding logic gates. The storage circuitry 2812 may be implemented by registers or the like. In the illustrated example, the storage circuitry 2812 is distributed amongst the logic gate circuitry 2808 to facilitate access and increase execution speed.

The example FPGA circuitry 2800 of FIG. 28 also includes example Dedicated Operations Circuitry 2814. In this example, the Dedicated Operations Circuitry 2814 includes special purpose circuitry 2816 that may be invoked to implement commonly used functions to avoid the need to program those functions in the field. Examples of such special purpose circuitry 2816 include memory (e.g., DRAM) controller circuitry, PCIe controller circuitry, clock circuitry, transceiver circuitry, memory, and multiplier-accumulator circuitry. Other types of special purpose circuitry may be present. In some examples, the FPGA circuitry 2800 may also include example general purpose programmable circuitry 2818 such as an example CPU 2820 and/or an example DSP 2822. Other general purpose programmable circuitry 2818 may additionally or alternatively be present such as a GPU, an XPU, etc., that can be programmed to perform other operations.

Although FIGS. 27 and 28 illustrate two example implementations of the processor circuitry 2612 of FIG. 26, many other approaches are contemplated. For example, as mentioned above, modern FPGA circuitry may include an on-board CPU, such as one or more of the example CPU 2820 of FIG. 28. Therefore, the processor circuitry 2612 of FIG. 26 may additionally be implemented by combining the example microprocessor 2700 of FIG. 27 and the example FPGA circuitry 2800 of FIG. 28. In some such hybrid examples, a first portion of the machine readable instructions may be executed by one or more of the cores 2702 of FIG. 27, a second portion of the machine readable instructions may be executed by the FPGA circuitry 2800 of FIG. 28, and/or a third portion of the machine readable instructions may be executed by an ASIC. It should be understood that some or all of the circuitry may, thus, be instantiated at the same or different times. Some or all of the circuitry may be instantiated, for example, in one or more threads executing concurrently and/or in series. Moreover, in some examples, some or all of the circuitry may be implemented within one or more virtual machines and/or containers executing on the microprocessor.

In some examples, the processor circuitry 2612 of FIG. 26 may be in one or more packages. For example, the microprocessor 2700 of FIG. 27 and/or the FPGA circuitry 2800 of FIG. 28 may be in one or more packages. In some examples, an XPU may be implemented by the processor circuitry 2612 of FIG. 26, which may be in one or more packages. For example, the XPU may include a CPU in one package, a DSP in another package, a GPU in yet another package, and an FPGA in still yet another package.

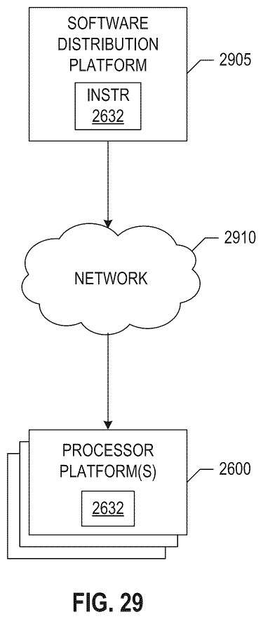

A block diagram illustrating an example software distribution platform 2905 to distribute software such as the example machine readable instructions 2632 of FIG. 26 to hardware devices owned and/or operated by third parties is illustrated in FIG. 29. The example software distribution platform 2905 may be implemented by any computer server, data facility, cloud service, etc., capable of storing and transmitting software to other computing devices. The third parties may be customers of the entity owning and/or operating the software distribution platform 2905. For example, the entity that owns and/or operates the software distribution platform 2905 may be a developer, a seller, and/or a licensor of software such as the example machine readable instructions 2632 of FIG. 26. The third parties may be consumers, users, retailers, OEMs, etc., who purchase and/or license the software for use and/or re-sale and/or sub-licensing. In the illustrated example, the software distribution platform 2905 includes one or more servers and one or more storage devices. The storage devices store the machine readable instructions 2632, which may correspond to the example machine readable instructions 2632 of FIG. 26, as described above. The one or more servers of the example software distribution platform 2905 are in communication with an example network 2910, which may correspond to any one or more of the Internet and/or any of the example networks described above. In some examples, the one or more servers are responsive to requests to transmit the software to a requesting party as part of a commercial transaction. Payment for the delivery, sale, and/or license of the software may be handled by the one or more servers of the software distribution platform and/or by a third party payment entity. The servers enable purchasers and/or licensors to download the machine readable instructions 2632 from the software distribution platform 2905. For example, the software, which may correspond to the example machine readable instructions 2632 of FIG. 26, may be downloaded to the example processor platform 2600, which is to execute the machine readable instructions 2632 to implement the systems and methods described herein. In some examples, one or more servers of the software distribution platform 2905 periodically offer, transmit, and/or force updates to the software (e.g., the example machine readable instructions 2632) to ensure improvements, patches, updates, etc., are distributed and applied to the software at the end user devices.

In some examples, rather than downloading machine readable instructions 2932 to a local processor platform 2900, a deployed model and/or patient data can be uploaded to execute remotely via the cloud-based platform 2905. In some examples, the example platform 2905 can host one or more models, accessible by the network 2910, and a processor platform 2600 can provide input to the model and receive a result, prediction, and/or other output.

From the foregoing, it will be appreciated that example systems, methods, apparatus, and articles of manufacture have been disclosed that enable model generation and deployment to drive processes for therapeutic prediction and treatment execution. Disclosed systems, methods, apparatus, and articles of manufacture improve the efficiency of using a computing device by enabling model generation and deployment to drive processes for therapeutic prediction and treatment execution. Disclosed systems, methods, apparatus, and articles of manufacture are accordingly directed to one or more improvement(s) in the operation of a machine such as a computer or other electronic and/or mechanical device.

Certain examples provide systems and methods to train and evaluate an AI model to predict an adverse risk event from fixed length patient history data. An example system and associated method include a submodule to extract patient blood test histories from electronic medical records, clean the histories and perform data quality checks. The example system and associated method include a label definition submodule instantiating an algorithm to assign a hepatitis adverse event grade to a set of blood test values (e.g., ALT, AST, TBILIRUBIN, ALKPHOS), and create a binary target label for the AI model. The example system and method include a feature engineering submodule to normalize blood test values to the upper limit of normal value (e.g., specific to patient, laboratory, and/or blood test, etc.). The example feature engineering submodule is to transform the normalized values to a discretized symbolic representation, such as a modified version of Symbolic Aggregate ApproXimation, etc. The example feature engineering submodule is to extract motifs as n-grams from the symbol series, and use the counts in recent patient history as features. The example system and method include a submodule to train and evaluate an AI model to dynamically predict immune-related hepatitis adverse event risk from fixed length blood test histories.

Certain examples provide systems and methods to build a classification model (e.g., a pneumonitis classification model, etc.) using a sequential procedure. An example system and method include preprocessing structured EHR and unstructured data tables. Patient timelines are aligned at the first ICI administration, for example. Lab measurements are aggregated over a time window (e.g., a 60-day time window, etc.) before the first ICI using statistics. Other features (e.g., conditions, smoking status, etc.) can use a different time window (e.g., a 1-year time window, etc.), for example.

The example system and method include finding patterns in the data to identify potential predictive features associated with development of ICI-related toxicities like pneumonitis, colitis, and hepatitis. In case of a small and noisy dataset, many data points are utilized. The data is split, with a first partition (e.g., a 90% partition, an 80% partition, a 95% partition, etc.) to identify candidate features based on associations between the pneumonitis label and the features.

The example system and method include, in each iteration of the procedure, deciding between two model versions, one with the original feature set (M1) and one extended with a candidate feature (M2). Nested cross-validation is performed on the first (e.g., 90%, etc.) partition, and the inner loop results are used to compare M1 and M2. A binomial test is performed to assess whether M2 is significantly better than M1.

The example system and method include, when M2 is significantly better in step 3), assessing whether M2 has better performance on the held out second partition (e.g., a 10% partition, 5% partition, 20% partition, etc.). A permutation test is performed to estimate the probability of observing a better performance just by random chance. This step acts as a safety measure to avoid overfitting to the first (e.g., 90%, etc.) data partition. If M2 has sufficiently better performance based on step 4), M2 is chosen.

The example system and method include continuing to test new candidate features until a desired model size is reached. The final model's performance on the outer loop is assessed. This way, a performance estimator with smaller variance is obtained, and variability in test predictions and model instability can be assessed. If the final model has promising performance, the model is evaluated on an external test set that is sufficiently large.

Certain examples provide systems and methods forming an automated framework to prepare multiple source electronic health record data for use in machine learning model training. The example method and associated system include preparing input data from multiple sources: This part of the framework is mainly concerned about cleaning and extracting features from multiple data sources. The step takes in raw, automatically derived EHR data in a data model format (e.g., an OMOP Data Model format, etc.) and multiple expert curated data sources for additional features and labels. The step is open for extensions and includes but is not restricted to modules for preparation of smoking history, drug administration, medical conditions, radiotherapy history, laboratory measurements and anthropometric data.

The example method and associated system include generating combined time dependent unrestricted intermediary outputs. This step is condensing the input data in a uniform format, retaining time stamps of the individual data items per patient. This intermediary step provides a plug-in possibility to modules preparing time dependent input data (not implemented) for sequence modeling algorithms and provides a flexible input for the aggregation step of the framework.

The example method and associated system include aggregating features for time independent models. This step is a target agnostic, highly configurable plug-in module for creating time independent, aggregated inputs for machine learning models. The step is open for extensions, configurable parameters include but are not restricted to prediction time point, length of aggregation, data sources to involve, feature types to involve.

Certain examples provide systems and methods for predictive model building for efficacy surrogate endpoint related to immune checkpoint inhibitor treatment. An example method and associated system include preparing input data from multiple sources. This part of the framework involves cleaning and extracting features from multiple data sources. The step takes in raw, automatically derived EHR data in OMOP Data Model format, for example, and multiple expert curated data sources for additional features. The example method and associated system include generating ground truth prediction labels such as Time on ICI treatment (TOT), Time to next treatment (after ICI discontinuation) (TNET), Overall Survival (OS), etc. The listed ground truth endpoints are generated on a continuous scale, expressed in days elapsed from an anchor point (patient timelines are alignment based on similarities in the ICI treatment course). The default anchor point is the first date of ICI treatment initiation. Generated ground truth can be used as is, or with modified granularity (elapsed weeks, months, years etc.) for training regression or survival analysis-based models. Discretization of the ground truth can be carried out for binary, or multiclass classification (e.g., responders vs. non-responders, 5-year survival, etc.).

The example method and associated system include model building on the generated feature matrix and the ground truth as a standalone module of the framework. Endpoints can be modeled separately. The modelling can be carried out hypothesis free, and with different machine learning algorithms, for example. A model building and selection workflow can be used to generate a predictive model for immune checkpoint inhibitor-related pneumonitis and a sequential procedure for model building.

Certain examples provide systems and methods for hepatitis prediction. An example system and associated method include input preparation through extraction of the relevant section of blood features from the Electronic Health Record (EHR) data tables received. These are measurements of liver biomarker concentration in the blood plasma (such as ALT, AST, Alkaline Phosphatase and Bilirubin) and other concentration values in the blood. This step is followed by putting the time series data into a single complex data structure, which is an efficient option to then continue by aggregating this information into the final data table, which is now ready for preprocessing and transformation steps. The aggregation step of the feature engineering consists of describing the time-series data of the blood particles with their mean, standard deviation, minimum and maximum. Lag features can also be created by taking the last liver biomarker measurements available before the treatment. The labels are created using a definition obtained from medical experts, which, for example, classified someone as positive when the level of at least one liver biomarker exceeded 3-times the upper limit of normal within a predefined window, negative otherwise. The date of the ICI treatment used in this scheme can be output from a different workflow.

The example system and method include dataset resampling. For example, the resulting dataset from step 1 is unbalanced, therefore random majority class us. is performed on the dataset if the goal is to maximize the recall value. If the F1-score is the subject of maximization, then the resampling step is disregarded.

The example system and method include training and prediction. For example, on the final data resulting from step 2, a model is trained, which is RF for recall maximization, and GB for F1-score maximization. The validation is carried out with Leave-One-Out Cross-Validation, where each sample is predicted individually with the rest as the training dataset.

### Brief Listing of Some Examples

Further aspects of the present disclosure are provided by the subject matter of the following clauses:

Example 1 is a method of configuring a treatment plan for a patient, the method including: a) loading a toxicity-related model for a selected toxicity associated with an immunotherapy, the toxicity-related model selected from a plurality of candidate models using a model selection criterion; b) determining a patient selection criterion to set a balance between precision and recall for action responsive to an output of the toxicity-related model, the output including a toxicity prediction; c) processing, using the toxicity-related model, healthcare data from a healthcare record for a given patient to generate the toxicity prediction; d) evaluating the toxicity prediction with respect to the patient and the patient selection criterion; e) when the toxicity prediction satisfies the patient selection criterion, generating a first indication and configuring the treatment plan to include the immunotherapy; f) when the toxicity prediction does not satisfy the patient selection criterion, generating a second indication and excluding the immunotherapy from the treatment plan; and g) outputting an order configuring the treatment plan for the patient based on the first indication or the second indication.

Example 2 is a method of configuring a cohort for a clinical trial, the method including: a) loading a toxicity-related model for a selected toxicity associated with an immunotherapy, the toxicity-related model selected from a plurality of candidate models using a model selection criterion; b) determining a patient selection criterion to set a balance between precision and recall for action responsive to an output of the toxicity-related model, the output including a toxicity prediction; c) processing, using the toxicity-related model, healthcare data from a healthcare record for a given patient to generate the toxicity prediction; d) evaluating the toxicity prediction with respect to the patient and the patient selection criterion; e) when the toxicity prediction satisfies the patient selection criterion, generating a first indication and configuring the treatment plan to include the immunotherapy; f) when the toxicity prediction does not satisfy the patient selection criterion, generating a second indication and excluding the immunotherapy from the treatment plan; and g) outputting an order configuring the treatment plan for the patient based on the first indication or the second indication.

Example 3 includes the method of any preceding clause, further including generating the toxicity-related model by forming features from an input data set obtained from healthcare records, the features iteratively compared through associated outcomes to form the model, the model tuned according to an analysis of precision and recall.

Example 4 includes the method of any preceding clause, wherein the patient selection criterion is a first patient selection criterion, the balance is a first balance, and the output is a first output, and further including: loading an efficacy-related model associated with an efficacy of the immunotherapy; determining a second patient selection criterion to set a second balance between precision and recall for action responsive to a second output of the efficacy-related model, the second output including an efficacy prediction; processing, using the efficacy-related model, healthcare data from a healthcare record for a given patient to generate the efficacy prediction; and evaluating the efficacy prediction with respect to the patient and the second patient selection criterion.

Example 5 includes the method of any preceding clause, further including: when the toxicity prediction satisfies the first patient selection criterion and the efficacy prediction satisfies the second patient selection criterion, generating the first indication and configuring the treatment plan to include the immunotherapy; and otherwise, generating the second indication and excluding the immunotherapy from the treatment plan.

Example 6 includes the method of any preceding clause, further including evaluating the toxicity prediction in comparison the efficacy prediction for the given patient to generate the respective first or second indication and configure the treatment plan to include or exclude the immunotherapy, respectively.

Example 7 includes the method of any preceding clause, wherein the efficacy is measured by patient survival or time on treatment.

Example 8 includes the method of any preceding clause, wherein configuring the treatment plan includes configuring the treatment plan for a plurality of selected patients to build a cohort for a clinical trial.

Example 9 includes the method of any preceding clause, wherein configuring the treatment plan further includes determining a schedule for application of the immunotherapy to the patient.

Example 10 includes the method of any preceding clause, wherein configuring the treatment plan further includes provisioning patient monitoring to evaluate the patient during execution of the treatment plan.

Example 11 includes the method of any preceding clause, wherein the patient selection criterion includes a threshold, the threshold defining a balance between false positives and false negatives for the toxicity-related model.

Example 12 includes the method of any preceding clause, wherein the selected toxicity is pneumonitis, colitis, or hepatitis, and wherein the toxicity prediction is i) a probability that the patient will suffer from the selected toxicity or ii) a probability that the patient will not suffer from the selected toxicity.

Example 13 includes the method of any preceding clause, wherein the healthcare data includes at least one of laboratory test results, diagnosis codes, or billing codes.

Example 14 includes the method of any preceding clause, further including aligning the healthcare data with respect to a reference point corresponding to an event in the healthcare record for the patient.

Example 15 includes the method of any preceding clause, further including re-evaluating the patient using updated healthcare data for the patient and an updated toxicity-related model.

Example 16 includes the method of any preceding clause, wherein the model selection criterion includes at least one of a high F1 score, a high recall, or a high precision.

Example 17 is a method of configuring a treatment plan for a patient, the method including: a) loading an efficacy-related model associated with an efficacy of an immunotherapy, the efficacy-related model selected from a plurality of candidate models using a model selection criterion; b) determining a patient selection criterion to set a balance between precision and recall for action responsive to an output of the efficacy-related model, the output including an efficacy prediction; c) processing, using the efficacy-related model, healthcare data from a healthcare record for a given patient to generate the efficacy prediction; d) evaluating the efficacy prediction with respect to the patient and the patient selection criterion; e) when the efficacy prediction satisfies the patient selection criterion, generating a first indication and configuring the treatment plan to include the immunotherapy; f) when the efficacy prediction does not satisfy the patient selection criterion, generating a second indication and excluding the immunotherapy from the treatment plan; and g) outputting an order configuring the treatment plan for the patient based on the first indication or the second indication.

Example 18 is a method of configuring a cohort for a clinical trial, the method including: a) loading an efficacy-related model associated with an efficacy of an immunotherapy, the efficacy-related model selected from a plurality of candidate models using a model selection criterion; b) determining a patient selection criterion to set a balance between precision and recall for action responsive to an output of the efficacy-related model, the output including an efficacy prediction; c) processing, using the efficacy-related model, healthcare data from a healthcare record for a given patient to generate the efficacy prediction; d) evaluating the efficacy prediction with respect to the patient and the patient selection criterion; e) when the efficacy prediction satisfies the patient selection criterion, generating a first indication and configuring the treatment plan to include the immunotherapy; f) when the efficacy prediction does not satisfy the patient selection criterion, generating a second indication and excluding the immunotherapy from the treatment plan; and g) outputting an order configuring the treatment plan for the patient based on the first indication or the second indication.

Example 19 includes the method of any preceding clause, further including generating the efficacy-related model by forming features from an input data set obtained from healthcare records, the features iteratively compared through associated outcomes to form the model, the model tuned according to an analysis of precision and recall.

Example 20 includes the method of any preceding clause, wherein the patient selection criterion is a first patient selection criterion, the balance is a first balance, and the output is a first output, and further including: loading a toxicity-related model for a selected toxicity associated with the immunotherapy; determining a second patient selection criterion to set a second balance between precision and recall for action responsive to a second output of the toxicity-related model, the second output including a toxicity prediction; processing, using the toxicity-related model, healthcare data from a healthcare record for a given patient to generate the toxicity prediction; and evaluating the toxicity prediction with respect to the patient and the second patient selection criterion.

Example 21 includes the method of any preceding clause, further including: when the efficacy prediction satisfies the first patient selection criterion and the toxicity prediction satisfies the second patient selection criterion, generating the first indication and configuring the treatment plan to include the immunotherapy; and otherwise, generating the second indication and excluding the immunotherapy from the treatment plan.

Example 22 includes the method of any preceding clause, further including evaluating the toxicity prediction in comparison the efficacy prediction for the given patient to generate the respective first or second indication and configure the treatment plan to include or exclude the immunotherapy, respectively.

Example 23 includes the method of any preceding clause, wherein the selected toxicity is pneumonitis, colitis, or hepatitis, and wherein the toxicity prediction is i) a probability that the patient will suffer from the selected toxicity or ii) a probability that the patient will not suffer from the selected toxicity.

Example 24 includes the method of any preceding clause, wherein configuring the treatment plan includes configuring the treatment plan for a plurality of selected patients to build a cohort for a clinical trial.

Example 25 includes the method of any preceding clause, wherein configuring the treatment plan further includes determining a schedule for application of the immunotherapy to the patient.

Example 26 includes the method of any preceding clause, wherein configuring the treatment plan further includes provisioning patient monitoring to evaluate the patient during execution of the treatment plan.

Example 27 includes the method of any preceding clause, wherein the patient selection criterion includes a threshold, the threshold defining a balance between false positives and false negatives for the efficacy-related model.

Example 28 includes the method of any preceding clause, wherein the healthcare data includes at least one of laboratory test results, diagnosis codes, or billing codes.

Example 29 includes the method of any preceding clause, further including aligning the healthcare data with respect to a reference point corresponding to an event in the healthcare record for the patient.

Example 30 includes the method of any preceding clause, further including re-evaluating the patient using updated healthcare data for the patient and an updated efficacy-related model.

Example 31 includes the method of any preceding clause, wherein the model selection criterion includes at least one of a high F1 score, a high recall, or a high precision.

Example 32 includes the method of any preceding clause, wherein the efficacy is measured by patient survival or time on treatment.

Example 33 is a method of configuring a treatment plan for a patient, the method including: a) generating a toxicity-related model for a selected toxicity associated with an immunotherapy by at least: i) train an initial model on a plurality of features generated from a plurality of prior healthcare data, the prior healthcare data arranged in time series and aligned with respect to a reference point; ii) iteratively add and remove each of a set of additional features to adjust the initial model into an adjusted model and evaluate a resulting precision and recall associated with an output of the adjusted model with respect to a model selection criterion; and iii) deploy the adjusted model as the toxicity-related model when the model selection criterion is satisfied; b) determining a patient selection criterion to set a balance between precision and recall for action responsive to an output of the toxicity-related model, the output including a toxicity prediction; c) processing, using the toxicity-related model, healthcare data from a healthcare record for a given patient to generate the toxicity prediction; d) evaluating the toxicity prediction with respect to the patient and the patient selection criterion; e) when the toxicity prediction satisfies the patient selection criterion, generating a first indication and configuring the treatment plan to include the immunotherapy; f) when the toxicity prediction does not satisfy the patient selection criterion, generating a second indication and excluding the immunotherapy from the treatment plan; and g) outputting an order configuring the treatment plan for the patient based on the first indication or the second indication.

Example 34 is a method of configuring a cohort for a clinical trial, the method including: a) generating a toxicity-related model for a selected toxicity associated with an immunotherapy by at least: i) train an initial model on a plurality of features generated from a plurality of prior healthcare data, the prior healthcare data arranged in time series and aligned with respect to a reference point; ii) iteratively add and remove each of a set of additional features to adjust the initial model into an adjusted model and evaluate a resulting precision and recall associated with an output of the adjusted model with respect to a model selection criterion; and iii) deploy the adjusted model as the toxicity-related model when the model selection criterion is satisfied; b) determining a patient selection criterion to set a balance between precision and recall for action responsive to an output of the toxicity-related model, the output including a toxicity prediction; c) processing, using the toxicity-related model, healthcare data from a healthcare record for a given patient to generate the toxicity prediction; d) evaluating the toxicity prediction with respect to the patient and the patient selection criterion; e) when the toxicity prediction satisfies the patient selection criterion, generating a first indication and configuring the treatment plan to include the immunotherapy; f) when the toxicity prediction does not satisfy the patient selection criterion, generating a second indication and excluding the immunotherapy from the treatment plan; and g) outputting an order configuring the treatment plan for the patient based on the first indication or the second indication.

Example 35 includes the method of any preceding clause, further including iteratively evaluating the model by partitioning the features into inner and outer loops and comparing the loops according to the model selection criterion.

Example 36 includes the method of any preceding clause, wherein the patient selection criterion is a first patient selection criterion, the balance is a first balance, and the output is a first output, and further including: loading an efficacy-related model associated with an efficacy of the immunotherapy; determining a second patient selection criterion to set a second balance between precision and recall for action responsive to a second output of the efficacy-related model, the second output including an efficacy prediction; processing, using the efficacy-related model, healthcare data from a healthcare record for a given patient to generate the efficacy prediction; and evaluating the efficacy prediction with respect to the patient and the second patient selection criterion.

Example 37 includes the method of any preceding clause, further including: when the toxicity prediction satisfies the first patient selection criterion and the efficacy prediction satisfies the second patient selection criterion, generating the first indication and configuring the treatment plan to include the immunotherapy; and otherwise, generating the second indication and excluding the immunotherapy from the treatment plan.

Example 38 includes the method of any preceding clause, further including evaluating the toxicity prediction in comparison the efficacy prediction for the given patient to generate the respective first or second indication and configure the treatment plan to include or exclude the immunotherapy, respectively.

Example 39 includes the method of any preceding clause, wherein the efficacy is measured by patient survival or time on treatment.

Example 40 includes the method of any preceding clause, wherein configuring the treatment plan includes configuring the treatment plan for a plurality of selected patients to build a cohort for a clinical trial.

Example 41 includes the method of any preceding clause, wherein configuring the treatment plan further includes scheduling application of the immunotherapy to the patient.

Example 42 includes the method of any preceding clause, wherein configuring the treatment plan further includes provisioning patient monitoring to evaluate the patient during execution of the treatment plan.

Example 43 includes the method of any preceding clause, wherein the patient selection criterion includes a threshold, the threshold defining a balance between false positives and false negatives for the toxicity-related model.

Example 44 includes the method of any preceding clause, wherein the selected toxicity is pneumonitis, colitis, or hepatitis, and wherein the toxicity prediction is i) a probability that the patient will suffer from the selected toxicity or ii) a probability that the patient will not suffer from the selected toxicity.

Example 45 includes the method of any preceding clause, wherein the healthcare data includes at least one of laboratory test results, diagnosis codes, or billing codes.

Example 46 includes the method of any preceding clause, further including aligning the healthcare data with respect to a reference point corresponding to an event in the healthcare record for the patient.

Example 47 includes the method of any preceding clause, further including re-evaluating the patient using updated healthcare data for the patient and an updated toxicity-related model.

Example 48 includes the method of any preceding clause, wherein the model selection criterion includes at least one of a high F1 score, a high recall, or a high precision.

Example 49 includes the method of any preceding clause, further including deploying the toxicity-related model in a tool with an interface to facilitate gathering of patient healthcare data and interaction with the toxicity-related model.

Example 50 is a method of configuring a treatment plan for a patient, the method including: a) generating an efficacy-related model predictive of an efficacy of an immunotherapy by at least: i) train an initial model on a plurality of features generated from a plurality of prior healthcare data, the prior healthcare data arranged in time series and aligned with respect to a reference point; ii) iteratively add and remove each of a set of additional features to adjust the initial model into an adjusted model and evaluate a resulting precision and recall associated with an output of the adjusted model with respect to a model selection criterion; and iii) deploy the adjusted model as the efficacy-related model when the model selection criterion is satisfied; b) determining a patient selection criterion to set a balance between precision and recall for action responsive to an output of the efficacy-related model, the output including an efficacy prediction; c) processing, using the efficacy-related model, healthcare data from a healthcare record for a given patient to generate the efficacy prediction; d) evaluating the efficacy prediction with respect to the patient and the patient selection criterion; e) when the efficacy prediction satisfies the patient selection criterion, generating a first indication and configuring the treatment plan to include the immunotherapy; f) when the efficacy prediction does not satisfy the patient selection criterion, generating a second indication and excluding the immunotherapy from the treatment plan; and g) outputting an order configuring the treatment plan for the patient based on the first indication or the second indication.

Example 51 is a method of configuring a cohort for a clinical trial, the method including: a) generating an efficacy-related model predictive of an efficacy of an immunotherapy by at least: i) train an initial model on a plurality of features generated from a plurality of prior healthcare data, the prior healthcare data arranged in time series and aligned with respect to a reference point; ii) iteratively add and remove each of a set of additional features to adjust the initial model into an adjusted model and evaluate a resulting precision and recall associated with an output of the adjusted model with respect to a model selection criterion; and iii) deploy the adjusted model as the efficacy-related model when the model selection criterion is satisfied; b) determining a patient selection criterion to set a balance between precision and recall for action responsive to an output of the efficacy-related model, the output including an efficacy prediction; c) processing, using the efficacy-related model, healthcare data from a healthcare record for a given patient to generate the efficacy prediction; d) evaluating the efficacy prediction with respect to the patient and the patient selection criterion; e) when the efficacy prediction satisfies the patient selection criterion, generating a first indication and configuring the treatment plan to include the immunotherapy; f) when the efficacy prediction does not satisfy the patient selection criterion, generating a second indication and excluding the immunotherapy from the treatment plan; and g) outputting an order configuring the treatment plan for the patient based on the first indication or the second indication.

Example 52 includes the method of any preceding clause, further including iteratively evaluating the model by partitioning the features into inner and outer loops and comparing the loops according to the model selection criterion.

Example 53 includes the method of any preceding clause, wherein the patient selection criterion is a first patient selection criterion, the balance is a first balance, and the output is a first output, and further including: loading a toxicity-related model for a selected toxicity associated with the immunotherapy; determining a second patient selection criterion to set a second balance between precision and recall for action responsive to a second output of the toxicity-related model, the second output including a toxicity prediction; processing, using the toxicity-related model, healthcare data from a healthcare record for a given patient to generate the toxicity prediction; and evaluating the toxicity prediction with respect to the patient and the second patient selection criterion.

Example 54 includes the method of any preceding clause, further including: when the efficacy prediction satisfies the first patient selection criterion and the toxicity prediction satisfies the second patient selection criterion, generating the first indication and configuring the treatment plan to include the immunotherapy; and otherwise, generating the second indication and excluding the immunotherapy from the treatment plan.

Example 55 includes the method of any preceding clause, further including evaluating the toxicity prediction in comparison the efficacy prediction for the given patient to generate the respective first or second indication and configure the treatment plan to include or exclude the immunotherapy, respectively.

Example 56 includes the method of any preceding clause, wherein the selected toxicity is pneumonitis, colitis, or hepatitis, and wherein the toxicity prediction is i) a probability that the patient will suffer from the selected toxicity or ii) a probability that the patient will not suffer from the selected toxicity.

Example 57 includes the method of any preceding clause, wherein configuring the treatment plan includes configuring the treatment plan for a plurality of selected patients to build a cohort for a clinical trial.

Example 58 includes the method of any preceding clause, wherein configuring the treatment plan further includes scheduling application of the immunotherapy to the patient.

Example 59 includes the method of any preceding clause, wherein configuring the treatment plan further includes provisioning patient monitoring to evaluate the patient during execution of the treatment plan.

Example 60 includes the method of any preceding clause, wherein the patient selection criterion includes a threshold, the threshold defining a balance between false positives and false negatives for the efficacy-related model.

Example 61 includes the method of any preceding clause, wherein the healthcare data includes at least one of laboratory test results, diagnosis codes, or billing codes.

Example 62 includes the method of any preceding clause, including aligning the healthcare data with respect to a reference point corresponding to an event in the healthcare record for the patient.

Example 63 includes the method of any preceding clause, further including re-evaluating the patient using updated healthcare data for the patient and an updated efficacy-related model.

Example 64 includes the method of any preceding clause, wherein the model selection criterion includes at least one of a high F1 score, a high recall, or a high precision.

Example 65 includes the method of any preceding clause, wherein the efficacy is measured by patient survival or time on treatment.

Example 66 includes the method of any preceding clause, further including deploying the efficacy-related model in a tool with an interface to facilitate gathering of patient healthcare data and interaction with the efficacy-related model.

Example 67 is a computer-readable medium including instructions which, when executed, cause processor circuitry to implement the method of any preceding clause.

Example 68 is an apparatus comprising: memory; instructions; and processor circuitry to implement the method of any preceding clause.

As disclosed and described herein, processor circuitry provides a means for processing (e.g., including means for processing input data, means for training models, means for selecting, means for deploying, means for generating, means for evaluating, means for triggering, means for ordering, means for displaying, etc.) and memory circuitry provides a means for storing. As described above, various circuitry can be implemented by the processor circuitry and by the memory circuitry.

The following claims are hereby incorporated into this Detailed Description by this reference. Although certain example systems, methods, apparatus, and articles of manufacture have been disclosed herein, the scope of coverage of this patent is not limited thereto. On the contrary, this patent covers all systems, methods, apparatus, and articles of manufacture fairly falling within the scope of the claims of this patent.

1. A computer-implemented method of configuring a treatment plan for a patient, the method comprising:
a) loading, by executing an instruction using processor circuitry, a toxicity-related model for a selected toxicity associated with an immunotherapy, the toxicity-related model selected from a plurality of candidate models using a model selection criterion;
b) determining, by executing an instruction using processor circuitry, a patient selection criterion to set a balance between precision and recall for action responsive to an output of the toxicity-related model, the output including a toxicity prediction;
c) processing, using the toxicity-related model, healthcare data from a healthcare record for a given patient to generate the toxicity prediction;
d) evaluating, by executing an instruction using processor circuitry, the toxicity prediction with respect to the patient and the patient selection criterion;
e) when the toxicity prediction satisfies the patient selection criterion, generating, by executing an instruction using processor circuitry, a first indication and configuring the treatment plan to include the immunotherapy;
f) when the toxicity prediction does not satisfy the patient selection criterion, generating, by executing an instruction using processor circuitry, a second indication and excluding the immunotherapy from the treatment plan; and
g) outputting, by executing an instruction using processor circuitry, an order configuring the treatment plan for the patient based on the first indication or the second indication.

2. The method of claim 1, further including generating the toxicity-related model by forming features from an input data set obtained from healthcare records, the features iteratively compared through associated outcomes to form the model, the model tuned according to an analysis of precision and recall.

3. The method of claim 1, wherein the patient selection criterion is a first patient selection criterion, the balance is a first balance, and the output is a first output, and further including:
loading an efficacy-related model associated with an efficacy of the immunotherapy;
determining a second patient selection criterion to set a second balance between precision and recall for action responsive to a second output of the efficacy-related model, the second output including an efficacy prediction;
processing, using the efficacy-related model, healthcare data from a healthcare record for a given patient to generate the efficacy prediction; and
evaluating the efficacy prediction with respect to the patient and the second patient selection criterion.

4. The method of claim 3, further including:
when the toxicity prediction satisfies the first patient selection criterion and the efficacy prediction satisfies the second patient selection criterion, generating the first indication and configuring the treatment plan to include the immunotherapy; and
otherwise, generating the second indication and excluding the immunotherapy from the treatment plan.

5. The method of claim 4, further including evaluating the toxicity prediction in comparison the efficacy prediction for the given patient to generate the respective first or second indication and configure the treatment plan to include or exclude the immunotherapy, respectively.

6. The method of claim 3, wherein the efficacy is measured by patient survival or time on treatment.

7. The method of claim 1, wherein configuring the treatment plan includes configuring the treatment plan for a plurality of selected patients to build a cohort for a clinical trial.

8. The method of claim 1, wherein configuring the treatment plan further includes determining a schedule for application of the immunotherapy to the patient.

9. The method of claim 1, wherein configuring the treatment plan further includes provisioning patient monitoring to evaluate the patient during execution of the treatment plan.

10. The method of claim 1, wherein the patient selection criterion includes a threshold, the threshold defining a balance between false positives and false negatives for the toxicity-related model.

11. The method of claim 1, wherein the selected toxicity is pneumonitis, colitis, or hepatitis, and wherein the toxicity prediction is i) a probability that the patient will suffer from the selected toxicity or ii) a probability that the patient will not suffer from the selected toxicity.

12. The method of claim 1, wherein the healthcare data includes at least one of laboratory test results, diagnosis codes, or billing codes.

13. The method of claim 12, further including aligning the healthcare data with respect to a reference point corresponding to an event in the healthcare record for the patient.

14. The method of claim 1, further including re-evaluating the patient using updated healthcare data for the patient and an updated toxicity-related model.

15. The method of claim 1, wherein the model selection criterion includes at least one of a high F1 score, a high recall, or a high precision.

16. A computer-readable medium comprising instructions which, when executed, cause processor circuitry to:
a) load a toxicity-related model for a selected toxicity associated with an immunotherapy, the toxicity-related model selected from a plurality of candidate models using a model selection criterion;
b) determine a patient selection criterion to set a balance between precision and recall for action responsive to an output of the toxicity-related model, the output including a toxicity prediction;
c) process, using the toxicity-related model, healthcare data from a healthcare record for a given patient to generate the toxicity prediction;
d) evaluate the toxicity prediction with respect to the patient and the patient selection criterion;
e) when the toxicity prediction satisfies the patient selection criterion, generate a first indication and configure the treatment plan to include the immunotherapy;
f) when the toxicity prediction does not satisfy the patient selection criterion, generate a second indication and exclude the immunotherapy from the treatment plan; and
g) output an order configuring the treatment plan for the patient based on the first indication or the second indication.

17. An apparatus comprising:
memory;
instructions; and
processor circuitry to:
a) load a toxicity-related model for a selected toxicity associated with an immunotherapy, the toxicity-related model selected from a plurality of candidate models using a model selection criterion;
b) determine a patient selection criterion to set a balance between precision and recall for action responsive to an output of the toxicity-related model, the output including a toxicity prediction;
c) process, using the toxicity-related model, healthcare data from a healthcare record for a given patient to generate the toxicity prediction;
d) evaluate the toxicity prediction with respect to the patient and the patient selection criterion;
e) when the toxicity prediction satisfies the patient selection criterion, generate a first indication and configure the treatment plan to include the immunotherapy;
f) when the toxicity prediction does not satisfy the patient selection criterion, generate a second indication and exclude the immunotherapy from the treatment plan; and
g) output an order configuring the treatment plan for the patient based on the first indication or the second indication.

18-70. (canceled)

71. The apparatus of claim 17, wherein the processor circuitry is to generate the toxicity-related model by forming features from an input data set obtained from healthcare records, the features iteratively compared through associated outcomes to form the model, the model tuned according to an analysis of precision and recall.

72. The apparatus of claim 17, wherein the patient selection criterion is a first patient selection criterion, the balance is a first balance, and the output is a first output, and wherein the processor circuitry is to:
load an efficacy-related model associated with an efficacy of the immunotherapy;
determine a second patient selection criterion to set a second balance between precision and recall for action responsive to a second output of the efficacy-related model, the second output including an efficacy prediction;
process, using the efficacy-related model, healthcare data from a healthcare record for a given patient to generate the efficacy prediction; and
evaluate the efficacy prediction with respect to the patient and the second patient selection criterion.

73. The apparatus of claim 19, wherein the processor is to:
when the toxicity prediction satisfies the first patient selection criterion and the efficacy prediction satisfies the second patient selection criterion, generate the first indication and configuring the treatment plan to include the immunotherapy; and
otherwise, generate the second indication and exclude the immunotherapy from the treatment plan.
